SEQUENCE LISTING (113 sequences)

SEQ ID NO: 1 - protein, 128 aa, synthetic construct
QVQLQESGPGVVKPSGTLSLTCAISGGSIGSGGSIRSTRWWSWVRQSPGKGLEWIGEIYHSGSTNYNPSLKSRVTISLDKSRNHFSLRLNSVTAADTAVYYCARQDYGDSGDWYFDLWGKGTMVTVSS

SEQ ID NO: 2 - protein, 12 aa, synthetic construct
SGGSIRSTRWWS

SEQ ID NO: 3 - protein, 16 aa, synthetic construct
EIYHSGSTNYNPSLKS

SEQ ID NO: 4 - protein, 13 aa, synthetic construct
QDYGDSGDWYFDL

SEQ ID NO: 5 - protein, 110 aa, synthetic construct
NFMLTQPHSVSESPGKTVTISCTRSSGSIASNSVQWYQQRPGSSPTTVIYEDNQRPSGVPDRFSGSIDSSSNSASLTVSGLKTEDEADYYCQSSDSSAVVFGSGTKLTVL

SEQ ID NO: 6 - protein, 13 aa, synthetic construct
TRSSGSIASNSVQ

SEQ ID NO: 7 - protein, 7 aa, synthetic construct
EDNQRPS

SEQ ID NO: 8 - protein, 9 aa, synthetic construct
QSSDSSAVV

SEQ ID NO: 9 - protein, 119 aa, synthetic construct
EVQLVQSGAEVKKPGASVKVSCKASGYRFTSYGISWVRQAPGQGLEWMGWISAYNGNTNYAQKLQGRVTMTTDTSTNTAYMELRSLRSDDTAVYYCARDADYSSGSGYWGQGTLVTVSS

SEQ ID NO: 10 - protein, 5 aa, synthetic construct
SYGIS

SEQ ID NO: 11 - protein, 16 aa, synthetic construct
WISAYNGNTNYAQKLQ

SEQ ID NO: 12 - protein, 10 aa, synthetic construct
DADYSSGSGY

SEQ ID NO: 13 - protein, 108 aa, synthetic construct
SYELTQPPSVSVSPGQTARITCSGDALPKQYAYWYQQKPGQAPVMVIYKDTERPSGIPERFSGSSSGTKVTLTISGVQAEDEADYYCQSADNSITYRVFGGGTKVTVL

SEQ ID NO: 14 - protein, 11 aa, synthetic construct
SGDALPKQYAY

SEQ ID NO: 15 - protein, 7 aa, synthetic construct
KDTERPS

SEQ ID NO: 16 - protein, 11 aa, synthetic construct
QSADNSITYRV

SEQ ID NO: 17 - protein, 119 aa, synthetic construct
QVQLVQSGAEVKKPGASVRVSCKASGYTLTSYYIHWVRQAPGQGLEWMGIINPRGATISYAQKFQGRVTMTRDTSTSTVYMELRNLKSEDTALYYCATAGIYGFDFDYWGRGTLVTVSS

SEQ ID NO: 18 - protein, 5 aa, synthetic construct
SYYIH

SEQ ID NO: 19 - protein, 17 aa, synthetic construct
IINPRGATISYAQKFQG

SEQ ID NO: 20 - protein, 10 aa, synthetic construct
AGIYGFDFDY

SEQ ID NO: 21 - protein, 111 aa, synthetic construct
QSALTQPASVSGSPGQSITISCTGTSNDVGGYNYVSWYQHHPGKAPKLIIYDVTNRPSGVSDRFSGSKSGNTASLTISGLLAEDEGDYYCSSYTIVTNFEVLFGGGTKLTV

SEQ ID NO: 22 - protein, 14 aa, synthetic construct
TGTSNDVGGYNYVS

SEQ ID NO: 23 - protein, 7 aa, synthetic construct
DVTNRPS

SEQ ID NO: 24 - protein, 12 aa, synthetic construct
SSYTIVTNFEVL

SEQ ID NO: 25 - protein, 127 aa, synthetic construct
QVQLQESGPGLVKPSQTLSLTCTVSGGSISSGAYYWSWIRQHPGKGLEWIGYIYYNGNTYYNPSLRSLVTISVDASKNQFSLKLSSVTAADTAVYYCARASDYVWGGYRYMDAFDIWGRGTLITVSS

SEQ ID NO: 26 - protein, 7 aa, synthetic construct
SGAYYWS

SEQ ID NO: 27 - protein, 16 aa, synthetic construct
YIYYNGNTYYNPSLRS

SEQ ID NO: 28 - protein, 17 aa, synthetic construct
ASDYVWGGYRYMDAFDI

SEQ ID NO: 29 - protein, 112 aa, synthetic construct
QSVLTQPPSASGTPGQRVTISCSGSNSNIGSNSVNWYQQLPGTAPKLLIYGNNQRPSGVPDRFSGSKSGTSASLAISGLQSENEADYYCAAWDDSLNGPVFGRGTKVTVLGE

SEQ ID NO: 30 - protein, 13 aa, synthetic construct
SGSNSNIGSNSVN

SEQ ID NO: 31 - protein, 7 aa, synthetic construct
GNNQRPS

SEQ ID NO: 32 - protein, 10 aa, synthetic construct
AAWDDSLNGP

SEQ ID NO: 33 - protein, 116 aa, synthetic construct
QIQLQQSGPELVKPGASVKISCKASGYTFTDYYINWVKQKPGQGLEWIGWIYPGSVNTKYNEKFRGKATLTVDTSSSTAYIQLSGLTSEDTAVYFCARYSNWFFDVWGAGTAVTVS

SEQ ID NO: 34 - protein, 5 aa, synthetic construct
DYYIN

SEQ ID NO: 35 - protein, 17 aa, synthetic construct
WIYPGSVNTKYNEKFRG

SEQ ID NO: 36 - protein, 8 aa, synthetic construct
YSNWFFDV

SEQ ID NO: 37 - protein, 108 aa, synthetic construct
DIVMTQSPSSLSASLGERVSLTCRASQEISGYLSWLQQKPDGTIKRLIYAASTLDSGVPKRFSGSRSGSDYSLSISSLESEDFADYYCLQYASYPYTFGGGTKLEIKR

SEQ ID NO: 38 - protein, 11 aa, synthetic construct
RASQEISGYLS

SEQ ID NO: 39 - protein, 7 aa, synthetic construct
AASTLDS

SEQ ID NO: 40 - protein, 9 aa, synthetic construct
LQYASYPYT

SEQ ID NO: 41 - protein, 446 aa, synthetic construct
QVQLVQSGAEVKKPGASVKVSCKASGYTFTDYYINWVRQAPGQGLEWMGWIYPGSVNTKYAQKFQGRVTMTVDTSTSTAYMELSSLRSEDTAVYYCARYSNWFFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 117 aa, synthetic construct
QVQLVQSGAEVKKPGASVKVSCKASGYTFTDYYINWVRQAPGQGLEWMGWIYPGSVNTKYAQKFQGRVTMTVDTSTSTAYMELSSLRSEDTAVYYCARYSNWFFDVWGQGTTVTVSS

SEQ ID NO: 43 - protein, 17 aa, synthetic construct
WIYPGSVNTKYAQKFQG

SEQ ID NO: 44 - protein, 446 aa, synthetic construct
QVQLVQSGAEVKKPGASVKISCKASGYTFTDYYINWVRQAPGQGLEWIGWIYPGSVNTKYAQKFQGRATLTVDTSTSTAYIELSSLRSEDTAVYYCARYSNWFFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 117 aa, synthetic construct
QVQLVQSGAEVKKPGASVKISCKASGYTFTDYYINWVRQAPGQGLEWIGWIYPGSVNTKYAQKFQGRATLTVDTSTSTAYIELSSLRSEDTAVYYCARYSNWFFDVWGQGTTVTVSS

SEQ ID NO: 46 - protein, 214 aa, synthetic construct
DIQMTQSPSSLSASVGDRVTITCRASQEISGYLSWLQQKPGKAPKLLIYAASTLDSGVPSRFSGSGSGTDYTLTISSLQPEDFATYYCLQYASYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 107 aa, synthetic construct
DIQMTQSPSSLSASVGDRVTITCRASQEISGYLSWLQQKPGKAPKLLIYAASTLDSGVPSRFSGSGSGTDYTLTISSLQPEDFATYYCLQYASYPYTFGQGTKLEIK

SEQ ID NO: 48 - protein, 214 aa, synthetic construct
DIQMTQSPSSLSASVGDRVTITCRASQEISGYLSWLQQKPGKAPKRLIYAASTLDSGVPSRFSGSGSGTDYTLTISSLQPEDFATYYCLQYASYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 107 aa, synthetic construct
DIQMTQSPSSLSASVGDRVTITCRASQEISGYLSWLQQKPGKAPKRLIYAASTLDSGVPSRFSGSGSGTDYTLTISSLQPEDFATYYCLQYASYPYTFGQGTKLEIK

SEQ ID NO: 50 - protein, 112 aa, synthetic construct
QVQLQQSDAELVKPGTSVKISCKASGYTFTDHAIHWLKQEPDQGLEWIGYISPGYGDIEYNEKFKGKATLTADKSSSTAYMQLSSLTSEDSAVYFCKVTTGYWGQGTTLTVS

SEQ ID NO: 51 - protein, 5 aa, synthetic construct
DHAIH

SEQ ID NO: 52 - protein, 17 aa, synthetic construct
YISPGYGDIEYNEKFKG

SEQ ID NO: 53 - protein, 4 aa, synthetic construct
TTGY

SEQ ID NO: 54 - protein, 113 aa, synthetic construct
DVLMTQTPLTLSVTIGQPASISCKSSQSLLNSDGKTYLNWLLQRPGQSPKRLIYLVSELDSGVPDRFTGSGSGTDFTLKISRVEAEDLGIYYCWQGTHFPHTFGAGTKLELKR

SEQ ID NO: 55 - protein, 16 aa, synthetic construct
KSSQSLLNSDGKTYLN

SEQ ID NO: 56 - protein, 7 aa, synthetic construct
LVSELDS

SEQ ID NO: 57 - protein, 9 aa, synthetic construct
WQGTHFPHT

SEQ ID NO: 58 - protein, 442 aa, synthetic construct
QVQLVQSGAEVKKPGASVKVSCKASGYTFTDHAIHWLRQAPGQGLEWMGYISPGYGDIEYAQKFQGRVTMTADTSTSTAYMELSSLRSEDTAVYYCKVTTGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 113 aa, synthetic construct
QVQLVQSGAEVKKPGASVKVSCKASGYTFTDHAIHWLRQAPGQGLEWMGYISPGYGDIEYAQKFQGRVTMTADTSTSTAYMELSSLRSEDTAVYYCKVTTGYWGQGTTVTVSS

SEQ ID NO: 60 - protein, 17 aa, synthetic construct
YISPGYGDIEYAQKFQG

SEQ ID NO: 61 - protein, 442 aa, synthetic construct
QVQLVQSGAEVKKPGASVKISCKASGYTFTDHAIHWLRQAPGQGLEWIGYISPGYGDIEYAQKFQGRATLTADTSTSTAYMELSSLRSEDTAVYYCKVTTGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 113 aa, synthetic construct
QVQLVQSGAEVKKPGASVKISCKASGYTFTDHAIHWLRQAPGQGLEWIGYISPGYGDIEYAQKFQGRATLTADTSTSTAYMELSSLRSEDTAVYYCKVTTGYWGQGTTLTVSS

SEQ ID NO: 63 - protein, 442 aa, synthetic construct
QVQLVQSGAEVKKPGASVKVSCKASGYTFTDHAIHWLRQAPGQGLEWMGYISPGYGDIEYAQKFQGRVTMTADKSTSTAYMELSSLRSEDTAVYYCKVTTGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 113 aa, synthetic construct
QVQLVQSGAEVKKPGASVKVSCKASGYTFTDHAIHWLRQAPGQGLEWMGYISPGYGDIEYAQKFQGRVTMTADKSTSTAYMELSSLRSEDTAVYYCKVTTGYWGQGTTVTVSS

SEQ ID NO: 65 - protein, 442 aa, synthetic construct
QVQLVQSGAEVKKPGASVKISCKASGYTFTDHAIHWLRQAPGQGLEWIGYISPGYGDIEYAQKFQGRATLTADKSTSTAYMELSSLRSEDTAVYYCKVTTGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 113 aa, synthetic construct
QVQLVQSGAEVKKPGASVKISCKASGYTFTDHAIHWLRQAPGQGLEWIGYISPGYGDIEYAQKFQGRATLTADKSTSTAYMELSSLRSEDTAVYYCKVTTGYWGQGTTLTVSS

SEQ ID NO: 67 - protein, 219 aa, synthetic construct
DVVMTQSPLSLPVTLGQPASISCRSSQSLLNSDGKTYLNWLQQRPGQSPRRLIYLVSELDSGVPDRFSGSGSGTDFTLKISRVEAEDVGVYYCWQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 112 aa, synthetic construct
DVVMTQSPLSLPVTLGQPASISCRSSQSLLNSDGKTYLNWLQQRPGQSPRRLIYLVSELDSGVPDRFSGSGSGTDFTLKISRVEAEDVGVYYCWQGTHFPHTFGQGTKLEIK

SEQ ID NO: 69 - protein, 117 aa, synthetic construct
EVKLVESGGGLVKPGGSLKLSCAASGFTFSSFGMSWVRQTPEKRLEWVATISGGGSNTYYPDSVKGRFTISRDNAKNNLYLQMTSLTSEDTALYYCTRIYDVAWFAYWGQGTLVTVS

SEQ ID NO: 70 - protein, 5 aa, synthetic construct
SFGMS

SEQ ID NO: 71 - protein, 17 aa, synthetic construct
TISGGGSNTYYPDSVKG

SEQ ID NO: 72 - protein, 9 aa, synthetic construct
IYDVAWFAY

SEQ ID NO: 73 - protein, 112 aa, synthetic construct
DIVLTQSPASLAVSLGQRATISCRASESVDNSGISFMNWFQQKPGQPPKLLIYAASNQGSGVPARFSGSGSGTDFSLNIHPMEEDDTAMYFCQQSKEVPWTFGGGTKLEIKR

SEQ ID NO: 74 - protein, 15 aa, synthetic construct
RASESVDNSGISFMN

SEQ ID NO: 75 - protein, 7 aa, synthetic construct
AASNQGS

SEQ ID NO: 76 - protein, 9 aa, synthetic construct
QQSKEVPWT

SEQ ID NO: 77 - protein, 447 aa, synthetic construct
EVQLVESGGGLVQPGGSLRLSCAASGFTFSSFGMSWVRQAPGKGLEWVATISGGGSNTYYADSVKGRFTISRDNSKNTLYLQMNSLRAEDTAVYYCTRIYDVAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 118 aa, synthetic construct
EVQLVESGGGLVQPGGSLRLSCAASGFTFSSFGMSWVRQAPGKGLEWVATISGGGSNTYYADSVKGRFTISRDNSKNTLYLQMNSLRAEDTAVYYCTRIYDVAWFAYWGQGTLVTVSS

SEQ ID NO: 79 - protein, 17 aa, synthetic construct
TISGGGSNTYYADSVKG

SEQ ID NO: 80 - protein, 218 aa, synthetic construct
DIQMTQSPSSLSASVGDRVTITCRASESVDNSGISFMNWFQQKPGKAPKLLIYAASNQGSGVPSRFSGSGSGTDFTLTISSLQPEDFATYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 111 aa, synthetic construct
DIQMTQSPSSLSASVGDRVTITCRASESVDNSGISFMNWFQQKPGKAPKLLIYAASNQGSGVPSRFSGSGSGTDFTLTISSLQPEDFATYYCQQSKEVPWTFGQGTKLEIK

SEQ ID NO: 82 - protein, 218 aa, synthetic construct
DIQLTQSPSSLSASVGDRATITCRASESVDNSGISFMNWFQQKPGKAPKLLIYAASNQGSGVPSRFSGSGSGTDFTLTISSMQPEDFATYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 111 aa, synthetic construct
DIQLTQSPSSLSASVGDRATITCRASESVDNSGISFMNWFQQKPGKAPKLLIYAASNQGSGVPSRFSGSGSGTDFTLTISSMQPEDFATYYCQQSKEVPWTFGQGTKLEIK

SEQ ID NO: 84 - protein, 119 aa, synthetic construct
EVQLVLSGGGFVQPGGSLKLSCAASGFTFSSYAMSWVRQNPERRLVWVATITGGGRNTYYPDSVKGRFTISRDNAKNTLYLQMSSLRSEDTAMYYCTRQGYDGYTWFAYWGQGTLVTVS

SEQ ID NO: 85 - protein, 5 aa, synthetic construct
SYAMS

SEQ ID NO: 86 - protein, 17 aa, synthetic construct
TITGGGRNTYYPDSVKG

SEQ ID NO: 87 - protein, 11 aa, synthetic construct
QGYDGYTWFAY

SEQ ID NO: 88 - protein, 112 aa, synthetic construct
DIVLTQSPTSLAVSLGQRATISCRASESVDNSGISFMNWFQQKPGQPPKLLIYAASNPGSGVPARFSGSGSGTDFSLNIHPMEEDDTAMYFCQQSKEVPWTFGGGTELEIKR

SEQ ID NO: 89 - protein, 15 aa, synthetic construct
RASESVDNSGISFMN

SEQ ID NO: 90 - protein, 7 aa, synthetic construct
AASNPGS

SEQ ID NO: 91 - protein, 9 aa, synthetic construct
QQSKEVPWT

SEQ ID NO: 92 - protein, 449 aa, synthetic construct
EVQLVESGGGLVQPGGSLRLSCAASGFTFSSYAMSWVRQAPGKGLEWVATITGGGRNTYYADSVKGRFTISRDNSKNTLYLQMNSLRAEDTAVYYCTRQGYDGYTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 93 - protein, 120 aa, synthetic construct
EVQLVESGGGLVQPGGSLRLSCAASGFTFSSYAMSWVRQAPGKGLEWVATITGGGRNTYYADSVKGRFTISRDNSKNTLYLQMNSLRAEDTAVYYCTRQGYDGYTWFAYWGQGTLVTVSS

SEQ ID NO: 94 - protein, 17 aa, synthetic construct
TITGGGRNTYYADSVKG

SEQ ID NO: 95 - protein, 218 aa, synthetic construct
DIQMTQSPSSLSASVGDRVTITCRASESVDNSGISFMNWFQQKPGKAPKLLIYAASNPGSGVPSRFSGSGSGTDFTLTISSLQPEDFATYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 111 aa, synthetic construct
DIQMTQSPSSLSASVGDRVTITCRASESVDNSGISFMNWFQQKPGKAPKLLIYAASNPGSGVPSRFSGSGSGTDFTLTISSLQPEDFATYYCQQSKEVPWTFGQGTKLEIK

SEQ ID NO: 97 - protein, 218 aa, synthetic construct
DIQLTQSPSSLSASVGDRATITCRASESVDNSGISFMNWFQQKPGKAPKLLIYAASNPGSGVPSRFSGSGSGTDFTLTISSMQPEDFATYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 111 aa, synthetic construct
DIQLTQSPSSLSASVGDRATITCRASESVDNSGISFMNWFQQKPGKAPKLLIYAASNPGSGVPSRFSGSGSGTDFTLTISSMQPEDFATYYCQQSKEVPWTFGQGTKLEIK

SEQ ID NO: 99 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLDGPSVFLFPPKPKDTLMISRTPEVTCVVVDVSDEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSDEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSDEDPEVKFNWYVDGVEVHNAKTKPREEQYNSTYRVVSVLTVLHQDWLNGKEYKCKVSNKAYPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKAEPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDPSVFLFPPKPKDTLMISRTPEVTCVVVDVSHEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 106 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGDPSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 108 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLDGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 110 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPDLLGDPSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 111 - protein, 107 aa, Homo sapiens [sequence omitted]

SEQ ID NO: 112 - protein, 16 aa, synthetic construct
RSSQSLLNSDGKTYLN

SEQ ID NO: 113 - protein, 8 aa, synthetic construct
WQGTHFPH

(19) United States
(12) Patent Application Publication
(10) Pub. No.: US 2026/0055187 A1
(43) Pub. Date: Feb. 26, 2026

(54) METHODS AND COMPOSITIONS FOR TREATING AUTOIMMUNE, ALLERGIC AND INFLAMMATORY DISEASES

(71) Applicant: Santa Ana Bio, Inc., Alameda, CA (US)

(72) Inventors: Peter Emtage, Lafayette, CA (US); Nancy Emtage, Alameda, CA (US)

(21) Appl. No.: 18/880,216

(22) PCT Filed: Jul. 6, 2023
(86) PCT No.: PCT/US2023/027013

§ 371 Date: Dec. 30, 2024

Related U.S. Application Data
(60) Provisional application No. 63/358,675, filed on Jul. 6, 2022.
(60) Provisional application No. 63/481,261, filed on Jan. 24, 2023.

(51) Int. Cl. C07K 16/28; A61K 47/68; A61P 37/06

(52) U.S. Cl. CPC C07K 16/2818; A61K 47/6849; A61P 37/06; C07K 2317/24; C07K 2317/40; C07K 2317/52; C07K 2317/565; C07K 2317/622; C07K 2317/72; C07K 2317/732; C07K 2317/734; C07K 2317/75; C07K 2317/76

(57) ABSTRACT

Disclosed herein are methods of treating autoimmune, allergic, and/or inflammatory diseases in a subject in need thereof, comprising administering a therapeutic agent comprising one or more antigen-binding sites on a cell involved in the autoimmune, allergic, and/or inflammatory diseases and depleting the cell.

### RELATED APPLICATIONS

The present application claims priority to U.S. Provisional Patent Application No. 63/358,675, filed Jul. 6, 2022; and U.S. Provisional Patent Application No. 63/481,261, filed Jan. 24, 2023, the entire contents of each which are hereby incorporated by reference for all purposes.

### BACKGROUND

The immune system is tightly controlled by various immune cells and co-stimulatory and co-inhibitory ligands and receptors. Inflammation is a normal physiological defense against pathogen infection and tissue damage and quickly ends under normal circumstances. However, in many chronic conditions, the inflammatory response continues and may lead to significant tissue and organ damage. Abnormal inflammatory response is shown to be associated with many chronic diseases, especially autoimmune diseases, in which immune cells act against self-protein. Allergy also results from dysregulation of the immune system, recognizing innocuous non-self-antigens. Mast cells and IgE antibodies take center stage in the allergic response, while rogue T or B cells are the primary actors in autoimmunity.

There remains a need for therapeutic agents and methods of treatment that fine-tune the activity of immune cells and the immune system that can be used alone or in combination for autoiinmurie, allergic, and inflammatory diseases.

### SUMMARY

The present application provides methods and compositions for treating autoimmune, allergic, and inflammatory diseases.

In one aspect provided are antibodies or antigen-binding fragments thereof that are capable of binding to PD-1, wherein the antibody or antigen binding fragment thereof (a) is a PD-1 agonist; (b) has enhanced binding to an Fc receptor; and/or (c) is non-fucosylated.

In some embodiments, the Fc receptor is an inhibitory Fc receptor. In some embodiments, the Fc receptor is an activating Fc receptor. In some embodiments, the Fc receptor is FcγRI. In some embodiments, the Fc receptor is FcγRII. In some embodiments, the Fc receptor is FcγRII.

In some embodiments, the antibody or antigen binding fragment thereof has enhanced effector function. In some embodiments, the effector function is antibody-dependent cellular cytotoxicity (ADCC), antibody-dependent cellular phagocytosis (ADCP), and complement dependent cytotoxicity (CDC).

In some embodiments, the antibody or antigen binding fragment thereof is capable of depleting a cell. In some embodiments, the cell is a PD-1 expressing cell. In some embodiments, the cell is an immune cell. In sone embodiments, the immune cell is a T cell, a B cell, a macrophage, a natural killer (NK) cell, a dendritic cell (DC), a monocyte, a neutrophil, a fibroblast, or an epithelial cell. In some embodiments, the immune cell is a T cell. In some embodiments, the immune cell is a B cell. In some embodiments, the immune cell is activated. In some embodiments, the immune cell is an antigen-activated T cell or B cell.

In some embodiments, the antibody or antigen binding fragment thereof is at least about 60%, about 75%, or about 90% non-fucosylated.

In some embodiments, the antibody or antigen binding fragment thereof comprises:
(a) (i) a heavy chain variable (VII) region comprising:
   heavy chain complementarity-determining region 1 (HCDR1) having the amino acid sequence of SEQ ID NO: 2, or a sequence differing in 1 or 2 amino acids therefrom,
   HCDR2 having the amino acid sequence of SEQ ID NO: 3, or a sequence differing in 1 or 2 amino acids therefrom, and
   HCDR3 having the amino acid sequence of SEQ ID NO: 4, or a sequence differing in 1 or 2 amino acids therefrom, and
   (ii) a light chain variable (VL) region comprising:
   light chain complementarity-determining region 1 (LCDR1) having the amino acid sequence of SEQ ID NO: 6, or a sequence differing in 1 or 2 amino acids therefrom,
   LCDR2 having the amino acid sequence of SEQ ID NO: 7, or a sequence differing in 1 or 2 amino acids therefrom, and
   LCDR3 having the amino acid sequence of SEQ ID NO: 8, or a sequence differing in 1 or 2 amino acids therefrom.
(b) (i) a VH region comprising:
   HCDR1 having the amino acid sequence of SEQ ID NO: 10, or a sequence differing in 1 or 2 amino acids therefrom,
   HCDR2 having the amino acid sequence of SEQ ID NO: 11, or a sequence differing in 1 or 2 amino acids therefrom, and
   HCDR3 having the amino acid sequence of SEQ ID NO: 12, or a sequence differing in 1 or 2 amino acids therefrom; and
   (ii) a VL region comprising:
   LCDR1 having the amino acid sequence of SEQ ID NO: 14, or a sequence differing in 1 or 2 amino acids therefrom,
   LCDR2 having the amino acid sequence of SEQ ID NO: 15, or a sequence differing in 1 or 2 amino acids therefrom, and
   LCDR3 having the amino acid sequence of SEQ ID NO: 16, or a sequence differing in 1 or 2 amino acids therefrom;
(c) (i) a VH region comprising:
   HCDR1 having the amino acid sequence of SEQ ID NO: 18, or a sequence differing in 1 or 2 amino acids therefrom,
   HCDR2 having the amino acid sequence of SEQ ID NO: 19, or a sequence differing in 1 or 2 amino acids therefrom, and
   HCDR3 having the amino acid sequence of SEQ ID NO: 20, or a sequence differing in 1 or 2 amino acids therefrom; and
   (ii) a VL region comprising:
   LCDR1 having the amino acid sequence of SEQ ID NO: 22, or a sequence differing in 1 or 2 amino acids therefrom,
   LCDR2 having the amino acid sequence of SEQ ID NO: 23, or a sequence differing in 1 or 2 amino acids therefrom, and
   LCDR3 having the amino acid sequence of SEQ ID NO: 24, or a sequence differing in 1 or 2 amino acids therefrom;
(d) (i) a VH region comprising:
   HCDR1 having the amino acid sequence of SEQ ID NO: 26, or a sequence differing in 1 or 2 amino acids therefrom,
   HCDR2 having the amino acid sequence of SEQ ID NO: 27, or a sequence differing in 1 or 2 amino acids therefrom, and
   HCDR3 having the amino acid sequence of SEQ ID NO: 28, or a sequence differing in 1 or 2 amino acids therefrom; and
   (ii) a VL region comprising:
   LCDR1 having the amino acid sequence of SEQ ID NO: 30, or a sequence differing in 1 or 2 amino acids therefrom,
   LCDR2 having the amino acid sequence of SEQ ID NO: 31, or a sequence differing in 1 or 2 amino acids therefrom, and
   LCDR3 having the amino acid sequence of SEQ ID NO: 32, or a sequence differing in 1 or 2 amino acids therefrom,
(e) (i) a VH region comprising:
   HCDR1 having the amino acid sequence of SEQ ID NO: 34, or a sequence differing in 1 or 2 amino acids therefrom,
   HCDR2 having the amino acid sequence of SEQ ID NO: 35, or a sequence differing in 1 or 2 amino acids therefrom, and
   HCDR3 having the amino acid sequence of SEQ ID NO: 36, or a sequence differing in 1 or 2 amino acids therefrom; and
   (ii) a VL region comprising:
   LCDR1 having the amino acid sequence of SEQ ID NO: 38, or a sequence differing in 1 or 2 amino acids therefrom,
   LCDR2 having the amino acid sequence of SEQ ID NO: 39, or a sequence differing in 1 or 2 amino acids therefrom, and
   LCDR3 having the amino acid sequence of SEQ ID NO: 40, or a sequence differing in 1 or 2 amino acids therefrom;
(f) (i) a VH region comprising:
   HCDR1 having the amino acid sequence of SEQ ID NO: 34, or a sequence differing in 1 or 2 amino acids therefrom,
   HCDR2 having the amino acid sequence of SEQ ID NO: 43, or a sequence differing in 1 or 2 amino acids therefrom, and
   HCDR3 having the amino acid sequence of SEQ ID NO: 36, or a sequence differing in 1 or 2 amino acids therefrom; and
   (ii) a VL region comprising:
   LCDR1 having the amino acid sequence of SEQ ID NO: 38, or a sequence differing in 1 or 2 amino acids therefrom,
   LCDR2 having the amino acid sequence of SEQ ID NO: 39, or a sequence differing in 1 or 2 amino acids therefrom, and
   LCDR3 having the amino acid sequence of SEQ ID NO: 40, or a sequence differing in 1 or 2 amino acids therefrom,
(g) (i) a VH region comprising:
   HCDR1 having the amino acid sequence of SEQ ID NO: 51, or a sequence differing in 1 or 2 amino acids therefrom,
   HCDR2 having the amino acid sequence of SEQ ID NO: 52, or a sequence differing in 1 or 2 amino acids therefrom, and
   HCDR3 having the amino acid sequence of SEQ ID NO: 53, or a sequence differing in 1 or 2 amino acids therefrom; and
   (ii) a VL region comprising:
   LCDR1 having the amino acid sequence of SEQ ID NO: 55, or a sequence differing in 1 or 2 amino acids therefrom,
   LCDR2 having the amino acid sequence of SEQ ID NO: 56, or a sequence differing in 1 or 2 amino acids therefrom, and
   LCDR3 having the amino acid sequence of SEQ ID NO: 57, or a sequence differing in 1 or 2 amino acids therefrom;
(h) (i) a VH region comprising:
   HCDR1 having the amino acid sequence of SEQ ID NO: 51, or a sequence differing in 1 or 2 amino acids therefrom,
   HCDR2 having the amino acid sequence of SEQ ID NO: 60, or a sequence differing in 1 or 2 amino acids therefrom, and
   HCDR3 having the amino acid sequence of SEQ ID NO: 53, or a sequence differing in 1 or 2 amino acids therefrom; and
   (ii) a VL region comprising:
   LCDR1 having the amino acid sequence of SEQ ID NO: 55, or a sequence differing in 1 or 2 amino acids therefrom,
   LCDR2 having the amino acid sequence of SEQ ID NO: 56, or a sequence differing in 1 or 2 amino acids therefrom, and
   LCDR3 having the amino acid sequence of SEQ ID NO: 57, or a sequence differing in 1 or 2 amino acids therefrom,
(i) (i) a VH region comprising:
   HCDR1 having the amino acid sequence of SEQ ID NO: 70, or a sequence differing in 1 or 2 amino acids therefrom,
   HCDR2 having the amino acid sequence of SEQ ID NO: 71, or a sequence differing in 1 or 2 amino acids therefrom, and
   HCDR3 having the amino acid sequence of SEQ ID NO: 72, or a sequence differing in 1 or 2 amino acids therefrom; and
   (ii) a VL region comprising:
   LCDR1 having the amino acid sequence of SEQ ID NO: 74, or a sequence differing in 1 or 2 amino acids therefrom,
   LCDR2 having the amino acid sequence of SEQ ID NO: 75, or a sequence differing in 1 or 2 amino acids therefrom, and
   LCDR3 having the amino acid sequence of SEQ ID NO: 76, or a sequence differing in 1 or 2 amino acids therefrom;
(j) (i) a VH region comprising:
   HCDR1 having the amino acid sequence of SEQ ID NO: 70, or a sequence differing in 1 or 2 amino acids therefrom,
   HCDR2 having the amino acid sequence of SEQ ID NO: 79, or a sequence differing in 1 or 2 amino acids therefrom, and
   HCDR3 having the amino acid sequence of SEQ ID NO: 72, or a sequence differing in 1 or 2 amino acids therefrom; and
   (ii) a VL region comprising:
   LCDR1 having the amino acid sequence of SEQ ID NO: 74, or a sequence differing in 1 or 2 amino acids therefrom,
   LCDR2 having the amino acid sequence of SEQ ID NO: 75, or a sequence differing in 1 or 2 amino acids therefrom, and
   LCDR3 having the amino acid sequence of SEQ ID NO: 76, or a sequence differing in 1 or 2 amino acids therefrom;
(k) (i) a VH region comprising:
   HCDR1 having the amino acid sequence of SEQ ID NO: 85, or a sequence differing in 1 or 2 amino acids therefrom,
   HCDR2 having the amino acid sequence of SEQ ID NO: 86, or a sequence differing in 1 or 2 amino acids therefrom, and
   HCDR3 having the amino acid sequence of SEQ ID NO: 87, or a sequence differing in 1 or 2 amino acids therefrom; and
   (ii) a VL region comprising:
   LCDR1 having the amino acid sequence of SEQ ID NO: 89, or a sequence differing in 1 or 2 amino acids therefrom,
   LCDR2 having the amino acid sequence of SEQ ID NC): 90, or a sequence differing in 1 or 2 amino acids therefrom, and
   LCDR3 having the amino acid sequence of SEQ ID NO: 91, or a sequence differing in 1 or 2 amino acids therefrom;
or
(l) (i) a VH region comprising:
   HCDR1 having the amino acid sequence of SEQ ID NO: 85, or a sequence differing in 1 or 2 amino acids therefrom,
   HCDR2 having the amino acid sequence of SEQ ID NO: 941 or a sequence differing in 1 or 2 amino acids therefrom, and
   HCDR3 having the amino acid sequence of SEQ ID NO: 87, or a sequence differing in 1 or 2 amino acids therefrom; and
   (ii) a VL region comprising:
   LCDR1 having the amino acid sequence of SEQ ID NO: 89, or a sequence differing in 1 or 2 amino acids therefrom,
   LCDR2 having the amino acid sequence of SEQ ID NO: 90, or a sequence differing in 1 or 2 amino acids therefrom, and
   LCDR3 having the amino acid sequence of SEQ ID NO: 91, or a sequence differing in 1 or 2 amino acids therefrom.

In some embodiments, the HCDR1, HCDR2, HCDR3, LCDR1, LCDR2, and LCDR3 comprise amino acid sequences that collectively differ by no more than two amino acid residues from the sequences of
(a) SEQ ID NO: 2, SEQ ID NO: 3, SEQ ID NO: 4, SEQ ID NO: 6, SEQ ID NO: 7, and SEQ LD NO: 8, respectively,
(b) SEQ ID NO: 10, SEQ ID NO: 11, SEQ ID NO: 12, SEQ ID NO: 14, SEQ ID NO: 15, and SEQ ID NO: 16, respectively;
(c) SEQ ID NO: 18, SEQ ID NO: 19, SEQ ID NO: 20, SEQ ID NO: 22, SEQ ID NO: 23, and SEQ ID NO: 24, respectively;
(d) SEQ ID NO: 26, SEQ ID NO: 27, SEQ ID NO: 28, SEQ ID NO: 30, SEQ ID NO: 31, and SEQ ID NO: 32, respectively;
(e) SEQ ID NO: 34, SEQ ID NO: 35, SEQ ID NO: 36, SEQ ID NO: 38, SEQ ID NO: 39, and SEQ ID NO: 40, respectively;
(f) SEQ ID NO: 34, SEQ LD NO: 43, SEQ ID NO: 36, SEQ ID NO: 38, SEQ ID NO: 39, and SEQ ID NO: 40, respectively;
(g) SEQ ID NO: 51, SEQ ID NO: 52, SEQ ID NO: 3, SEQ ID NO: 55, SEQ ID NO: 56, and SEQ ID NO: 57, respectively,
(h) SEQ ID NO: 51, SEQ ID NO: 60, SEQ ID NO: 53, SEQ ID NO: 55, SEQ ID NO: 56, and SEQ ID NO: 57, respectively;
(i) SEQ ID NO: 70, SEQ ID NO: 71, SEQ ID NO: 72, SEQ ID NO: 74, SEQ ID NO: 75, and SEQ ID NO: 76, respectively;
(j) SEQ ID NO: 70, SEQ ID NO: 79, SEQ ID NO: 72, SEQ ID NO: 74, SEQ ID NO: 75, and SEQ ID NO: 76, respectively;
(k) SEQ ID NO: 85, SEQ ID NO: 86, SEQ ID NO: 87, SEQ ID NO: 89, SEQ ID NO: 90, and SEQ ID NO: 91, respectively; or
(l) SEQ ID NO: 85, SEQ ID NO: 94, SEQ ID NO: 87, SEQ ID NO: 89, SEQ ID NO: 90, and SEQ ID NO: 91, respectively.

In some embodiments, the antibody or antigen binding fragment thereof comprises:
(a) (i) a VH region comprising:
   CDR-H1 comprising the amino acid sequence of SEQ ID NO: 2,
   CDR-H2 comprising the amino acid sequence of SEQ ID NO: 3, and
   CDR-H3 comprising the amino acid sequence of SEQ ID NO: 4; and
(ii) a VL, region comprising:
   CDR-L1 comprising the amino acid sequence of SEQ ID NO: 6,
   CDR-L2 comprising the amino acid sequence of SEQ ID NO: 7, and
   CDR-L3 comprising the amino acid sequence of SEQ ID NO: 8;
(b) (i) a VL region comprising:
   CDR-H1 comprising the amino acid sequence of SEQ ID NO: 10,
   CDR-H2 comprising the amino acid sequence of SEQ ID NO: 11, and
   CDR-H3 comprising the amino acid sequence of SEQ ID NO: 12; and
   (ii) a VL region comprising:
   CDR-L1 comprising the amino acid sequence of SEQ ID NO: 14,
   CDR-L2 comprising the amino acid sequence of SEQ ID NO: 15, and
   CDR-L3 comprising the amino acid sequence of SEQ ID NO: 16;
(c) (i) a VH region comprising:
   CDR-H1 comprising the amino acid sequence of SEQ ID NO: 18,
   CDR-H2 comprising the amino acid sequence of SEQ ID NO: 19, and
   CDR-H3 comprising the amino acid sequence of SEQ ID NO: 20; and
   (ii) a VL region comprising:
   CDR-L1 comprising the amino acid sequence of SEQ ID NO: 22,
   CDR-L2 comprising the amino acid sequence of SEQ ID NO: 23, and
   CDR-L3 comprising the amino acid sequence of SEQ ID NO: 24;
(d) (i) a VH region comprising:
   CDR-H1 comprising the amino acid sequence of SEQ ID NO: 26,
   CDR-H2 comprising the amino acid sequence of SEQ ID NO: 27, and
   CDR-H3 comprising the amino acid sequence of SEQ ID NO: 28; and
   (ii) a VL region comprising:
   CDR-L1 comprising the amino acid sequence of SEQ ID NO: 30,
   CDR-L2 comprising the amino acid sequence of SEQ ID NO: 31, and
   CDR-L3 comprising the amino acid sequence of SEQ ID NO: 32;
(e) (i) a VH region comprising:
   CDR-H1 comprising the amino acid sequence of SEQ LD NO: 34,
   CDR-H2 comprising the amino acid sequence of SEQ ID NO: 35, and
   CDR-H3 comprising the amino acid sequence of SEQ ID NO: 36; and
   (ii) a VL region comprising:
   CDR-L1 comprising the amino acid sequence of SEQ ID NO: 38,
   CDR-L2 comprising the amino acid sequence of SEQ ID NO: 39, and
   CDR-L3 comprising the amino acid sequence of SEQ ID NO: 40;
(f) (i) a VH region comprising:
   CDR-H1 comprising the amino acid sequence of SEQ ID NO: 34,
   CDR-H2 comprising the amino acid sequence of SEQ ID NO: 35, and
   CDR-H3 comprising the amino acid sequence of SEQ ID NO: 36; and
   (ii) a VL region comprising:
   CDR-L1 comprising the amino acid sequence of SEQ ID NO: 38,
   CDR-L2 comprising the amino acid sequence of SEQ ID NO: 39, and
   CDR-L3 comprising the amino acid sequence of SEQ ID NO: 40;
(g) (i) a VH region comprising:
   CDR-H1 comprising the amino acid sequence of SEQ ID) NO: 51,
   CDR-H2 comprising the amino acid sequence of SEQ ID NO: 52, and
   CDR-H3 comprising the amino acid sequence of SEQ LD NO: 53; and
   (ii) a VL region comprising:
   CDR-L1 comprising the amino acid sequence of SEQ ID NO: 55,
   CDR-L2 comprising the amino acid sequence of SEQ ID NO: 56, and
   CDR-L3 comprising the amino acid sequence of SEQ ID NO: 57;
(h) (i) a VH region comprising:
   CDR-H1 comprising the amino acid sequence of SEQ ID NO: 51,
   CDR-H2 comprising the amino acid sequence of SEQ ID NO: 60, and
   CDR-H3 comprising the amino acid sequence of SEQ ID NO: 53; and
   (ii) a VL region comprising:
   CDR-L1 comprising the amino acid sequence of SEQ ID NO: 55,
   CDR-L2 comprising the amino acid sequence of SEQ ID NO: 56, and
   CDR-L3 comprising the amino acid sequence of SEQ ID NO: 57;
(i) (i) a VH region comprising:
   CDR-H1 comprising the amino acid sequence of SEQ ID NO: 70,
   CDR-H2 comprising the amino acid sequence of SEQ ID NO: 71, and
   CDR-H3 comprising the amino acid sequence of SEQ ID NO: 72; and
   (ii) a VL region comprising:
   CDR-L1 comprising the amino acid sequence of SEQ ID NO: 74.
   CDR-L2 comprising the amino acid sequence of SEQ ID NO: 75, and
   CDR-L3 comprising the amino acid sequence of SEQ ID NO: 76;
(j) (i) a VH region comprising:
   CDR-H1 comprising the amino acid sequence of SEQ ID NO: 70,
   CDR-H2 comprising the amino acid sequence of SEQ ID NO: 79, and
   CDR-H3 comprising the amino acid sequence of SEQ ID NO: 72; and
   (ii) a VFL region comprising:
   CDR-L1 comprising the amino acid sequence of SEQ ID NO: 74,
   CDR-L2 comprising the amino acid sequence of SEQ ID NO: 75, and
   CDR-L3 comprising the amino acid sequence of SEQ ID NO: 76;
(k) (i) a VH region comprising:
   CDR-H1 comprising the amino acid sequence of SEQ ID NO: 85,
   CDR-H2 comprising the amino acid sequence of SEQ ID NO: 86, and
   CDR-H3 comprising the amino acid sequence of SEQ ID NO: 87; and
   (ii) a VL region comprising:
   CDR-L1 comprising the amino acid sequence of SEQ ID NO: 89,
   CDR-L2 comprising the amino acid sequence of SEQ ID NO: 90, and
   CDR-L3 comprising the amino acid sequence of SEQ ID NO: 91;
or
(l) (i) a VH region comprising:
   CDR-H1 comprising the amino acid sequence of SEQ ID NO: 85,
   CDR-H2 comprising the amino acid sequence of SEQ ID NO: 86, and
   CDR-H3 comprising the amino acid sequence of SEQ ID NO: 87; and
   (ii) a VL region comprising:
   CDR-L1 comprising the amino acid sequence of SEQ ID NO: 89.
   CDR-L2 comprising the amino acid sequence of SEQ ID NO: 90, and
   CDR-L3 comprising the amino acid sequence of SEQ ID NO: 91.

In some embodiments, the antibody or antigen binding fragment thereof comprises:
(i) a VH region comprising:
   CDR-H1 comprising the amino acid sequence of SEQ ID NO: 34,
   CDR-H2 comprising the amino acid sequence of SEQ ID NO: 43, and
   CDR-H3 comprising the amino acid sequence of SEQ ID NO: 36; and
(ii) a VL region comprising:
   CDR-L1 comprising the amino acid sequence of SEQ ID NO: 38.
   CDR-L2 comprising the amino acid sequence of SEQ ID NO: 39, and
   CDR-L3 comprising the amino acid sequence of SEQ ID NO: 40;
In some embodiments, the antibody or antigen binding fragment thereof comprises:
(a) a VH region comprising an amino acid sequence having at least 70%, 80%, 90%, 95%, 96%, 97%, 98%, or 99% sequence identity to the amino acid sequence of SEQ ID NO: 1 and a VL region comprising an amino acid sequence having at least 70%, 80%, 90%, 95%, 96%, 97%, 98%, or 99% sequence identity to the amino acid sequence of SEQ ID NO: 5;
(b) a VH region comprising an amino acid sequence having at least 70%, 80%, 90%, 95%, 96%, 97%, 98%, or 99% sequence identity to the amino acid sequence of SEQ ID NO: 9 and a VL region comprising an amino acid sequence having at least 70%, 80%, 90%, 95%, 96%, 97%, 98%, or 99% sequence identity to the amino acid sequence of SEQ ID NO: 13;
(c) a VH region comprising an amino acid sequence having at least 70%, 80%, 90%, 95%, 96%, 97%, 98%, or 99% sequence identity to the amino acid sequence of SEQ ID NO: 17 and a VL region comprising an amino acid sequence having at least 70%, 80%, 90%, 95%, 96%, 97%, 98%, or 99% sequence identity to the amino acid sequence of SEQ ID NO: 21;
(d) a VH region comprising an amino acid sequence having at least 70%, 80%, 90%, 95%, 96%, 97%, 98%, or 99% sequence identity to the amino acid sequence of SEQ ID NO: 25 and a VL, region comprising an amino acid sequence having at least 70%, 80%, 90%, 95%, 96%, 97%, 98%, or 99% sequence identity to the amino acid sequence of SEQ ID NO: 29;
(e) a VH region comprising an amino acid sequence having at least 70%, 80%, 90%, 95%, 96%, 97%, 98%, or 99% sequence identity to the amino acid sequence of SEQ ID NO: 33 and a VL region comprising an amino acid sequence having at least 70%, 80%, 90%, 95%, 96%, 97%, 98%, or 99% sequence identity to the amino acid sequence of SEQ ID NO: 37;
(f) a VH region comprising an amino acid sequence having at least 70%, 80%, 90%, 95%, 96%, 97%, 98%, or 99% sequence identity to the amino acid sequence of SEQ ID NO: 42 or 45 and a VL region comprising an amino acid sequence having at least 70%, 80%, 90%, 95%, 96%, 97%, 98%, or 99% sequence identity to the amino acid sequence of SEQ ID NO: 47 or 49;
(g) a VH region comprising an amino acid sequence having at least 70%, 80%, 90%, 95%, 96%, 97%, 98%, or 99% sequence identity to the amino acid sequence of SEQ ID NO: 50 and a VL region comprising an amino acid sequence having at least 70%, 80%, 90%, 95%, 96%, 97%, 98%, or 99% sequence identity to the amino acid sequence of SEQ ID NO: 54;
(h) a VH region comprising an amino acid sequence having at least 70%, 80%, 90%, 95%, 96%, 97%, 98%, or 99% sequence identity to the amino acid sequence of SEQ ID NO: 56, 62, 64, or 66 and a VL region comprising an amino acid sequence having at least 70%, 80⁰, 90%, 95%, 96%, 97%, 98%, or 99% sequence identity to the amino acid sequence of SEQ FD NO: 68;
(i) a VH region comprising an amino acid sequence having at least 70%, 80%, 90%, 95%, 96%, 97%, 98%, or 99% sequence identity to the amino acid sequence of SEQ ID NO: 69 and a VL region comprising an amino acid sequence having at least 70%, 80%, 90%, 95%, 960, 97%, 98%, or 99% sequence identity to the amino acid sequence of SEQ ID NO: 73;
(j) a VH region comprising an amino acid sequence having at least 70%, 80%, 90%, 95%, 96%, 97%, 98%, or 99% sequence identity to the amino acid sequence of SEQ ID NO: 78 and a VL region comprising an amino acid sequence having at least 70%, 80%, 90%, 95%, 96%, 97%, 98%, or 99% sequence identity to the amino acid sequence of SEQ ID NO: 81 or 83;
(k) a VH region comprising an amino acid sequence having at least 70%, 80%, 90%, 95%, 96%, 97%, 98%, or 99% sequence identity to the amino acid sequence of SEQ ID NO: 84 and a VL region comprising an amino acid sequence having at least 70%, 80%, 90%, 95%, 96%, 97%, 98%, or 99% sequence identity to the amino acid sequence of SEQ ID NO: 88;
or
(l) a VH region comprising an amino acid sequence having at least 70%, 80%, 90%, 95%, 96%, 97%, 98%, or 99% sequence identity to the amino acid sequence of SEQ ID NO: 93 and a VL region comprising an amino acid sequence having at least 70%, 80%, 90%, 95%, 96%, 97%, 98%, or 99% sequence identity to the amino acid sequence of SEQ ID NO: 96 or 98.

In some embodiments, the antibody or antigen binding fragment thereof comprises;
(a) a VH region having the amino acid sequence of SEQ ID NO: 1 and a VL region having the amino acid sequence of the amino acid sequence of SEQ ID NO: 5;
(b) a VH region having the amino acid sequence of the amino acid sequence of SEQ ID NO: 9 and a VL region having the amino acid sequence of the amino acid sequence of SEQ ID NO: 13,
(c) a VH region having the amino acid sequence of the amino acid sequence of SEQ ID NO: 17 and a VL region having the amino acid sequence of the amino acid sequence of SEQ ID NO: 21;
(d) a VII region having the amino acid sequence of the amino acid sequence of SEQ ID NO: 25 and a VL region having the amino acid sequence of the amino acid sequence of SEQ ID NO: 29;
(e) a VH region having the amino acid sequence of the amino acid sequence of SEQ ID NO: 33 and a VL region having the amino acid sequence of the amino acid sequence of SEQ ID NO: 37;
(f) a VH region having the amino acid sequence of the amino acid sequence of SEQ ID NO: 42 or 45 and a VL region having the amino acid sequence of the amino acid sequence of SEQ ID NO: 47 or 49;
(g) a VII region having the amino acid sequence of the amino acid sequence of SEQ ID NO: 50 and a ML region having the amino acid sequence of the amino acid sequence of SEQ ID NO: 54;
(h) a VH region having the amino acid sequence of the amino acid sequence of SEQ ED NO: 56, 62, 64, or 66 and a VL region having the amino acid sequence of the amino acid sequence of SEQ ID NO: 68;
(i) a VH region having the amino acid sequence of the amino acid sequence of SEQ ID NO 69 and a VL region having the amino acid sequence of the amino acid sequence of SEQ ID NO: 73,
(j) a VTI region having the amino acid sequence of the amino acid sequence of SEQ ID NO: 78 and a VL region having the amino acid sequence of the amino acid sequence of SEQ ID NO: 81 or 83;
(k) a VH region having the amino acid sequence of the amino acid sequence of SEQ ID NO: 84 and a VL region having the amino acid sequence of the amino acid sequence of SEQ ID NO: 88; or
(l) a VH region having the amino acid sequence of the amino acid sequence of SEQ ID NO: 93 and a VL region having the amino acid sequence of the amino acid sequence of SEQ ID NO: 96 or 98.

In some embodiments, the antibody or antigen binding fragment thereof comprises: a VH region having the amino acid sequence of the amino acid sequence of SEQ ID NO: 42 or 45 and a VL region having the amino acid sequence of the amino acid sequence of SEQ ID NO: 47 or 49.

In some embodiments, the antibody is a humanized antibody. In some embodiments, the antigen binding fragment is a Fab, a Fab′, a Fab2, a F(ab′)2, Fv, a single-chain Fv (scFv), or a diabody. In some embodiments, the antigen binding fragment is an scFv.

In some embodiments, the antibody or antigen binding fragment thereof comprises an antibody heavy chain constant region.

In some embodiments, the antibody heavy chain constant region is a human IgG heavy chain constant region. In some embodiments, the antibody heavy chain constant region is a human IgG1 heavy chain constant region. In some embodiments, the antibody heavy chain constant region comprises an amino acid sequence having at least 70%, 80%, 90%, 95%, 96%, 97%, 98%, or 99% sequence identity to the amino acid sequence of SEQ ID NO: 100. In some embodiments, the antibody heavy chain constant region has the amino acid sequence of SEQ ID NO: 100.

In some embodiments, the antibody heavy chain constant region comprises one or more mutations that enhance effector function. In some embodiments, the antibody heavy chain constant region is a human IgG1 heavy chain constant region comprising one of the following mutation(s): (a) E233D; (b) G236D; (c) G237D; (d) P238D; (e) S239D; (f) S267E; (g) H268D; (h) P271G; (i) L328Y, L328F, or L328E: (j) A330R; (k) I332E; (l) 236D and H268D; (m) S239D and H3268D, (n) S239D, H268D, L328Y, and I332E; (o) P238D and L328E; (p) G237D, P271 G, and A330R; (q) G237D, I268D, P271G, and A330R; (r) S267E and L328F; (s) S239D and S267E; (t) G236D and S276E, or (u) E233D, G237D, H268D, P271G, and A330R, or a combination thereof, wherein the numbering of the constant region is as per the EU index. In some embodiments, the antibody heavy chain constant region is a human IgG1 heavy chain constant region comprising the following mutations: S239D and H268D, wherein the numbering of the constant region is as per the EU index. In some embodiments, the antibody heavy chain constant region is a human IgG1 heavy chain constant region comprising the following mutations: E233D, G237D, H268D, P271G, and A330R, wherein the numbering of the constant region is as per the EU index.

In some embodiments, the antibody or antigen-binding fragment thereof is produced in a cell line
(a) having an alpha-1,6-fucosyltransferase (Fut8) knockout; or
(b) line overexpressing β1,4-N-acetylglucosaninyltransferase III (GnT-III) and optionally overexpressing Golgi μ-mannosidase II (ManII).

In some embodiments, the antibody or antigen-binding fragment is capable of binding to human PD-1. In some embodiments, the antibody or antigen-binding fragment is capable of binding to cynomnonguls PD-1.

In some embodiments, the antibody or antigen-binding fragment is capable of blocking PD-1/PD-L1 interaction.

In some embodiments, the antibody or antigen-binding fragment is capable of inhibiting T cell activation. In some embodiments, the antibody or antigen-binding fragment is capable of inhibiting T cell proliferation. In some embodiments, the antibody or antigen-binding fragment is capable of inhibiting IFN-γ secretion.

In some embodiments, the wherein the antibody or antigen-binding fragment thereof is conjugated to an agent. In some embodiments, the agent is a cytotoxic agent or label. In some embodiments, the agent is a therapeutic agent.

In another aspect, provided are compositions comprising an antibody or antigen binding fragment thereof described herein. In some embodiments, the antibody or antigen-binding fragment comprises an Fe region and N-glycoside-linked carbohydrate chains linked to the Fe region, optionally wherein less than 50% of the N-glycoside-linked carbohydrate chains contain a fucose residue. In some embodiments, substantially none of the N-glycoside-linked carbohydrate chains contain a fucose residue.

In another aspect, provided are polynucleotides encoding an antibody or antigen binding fragment thereof described herein.

In another aspect, provided are expression vectors comprising a polynucleotide described herein.

In another aspect, provided are host cell comprising a polynucleotide or an expression vector described herein. In some embodiments, the host cell is a mammalian or insect cell. In some embodiments, the host cell (a) comprises a Fut8 knockout; and/or (b) overexpresses GnT-III and optionally ManII.

In another aspect, provided are pharmaceutical compositions comprising an antibody or antigen binding fragment thereof described herein and a pharmaceutically acceptable carrier.

In another aspect, provided are methods of treating a disease or disorder in a subject in need thereof, comprising administering to the subject an effective amount of an antibody or antigen binding fragment thereof described herein. In some embodiments, the disease or disorder is an autoimmune disease. In some embodiments, the disease or disorder is an allergy. In some embodiments, the disease or disorder is an inflammatory disease.

### BRIEF DESCRIPTION OF THE DRAWINGS

FIG. 1 depicts the blockade of PD-1/PD-L1 interaction by disclosed PD-1 antibodies. The percent blocking was plotted against a standard curve of control PD-1 neutralizing antibody.

FIG. 2 shows that disclosed PD-1 antibodies suppressed IFN-γ secretion in a co-culture of peripheral blood mononuclear cells (PBMCs) from different donors, causing a mixed lymphocyte response (MLR).

FIG. 3 depicts the inhibition of the activation of Jurkat reporter T cells incubated with Raji-APC cells modified to express the FcγRII receptor by PD-1 antibodies with normal IgG1 Fc or modified Fc containing mutations to increase binding to FcγRII.

FIG. 4 depicts the inhibition of IL-2 secretion in Jurkat reporter T cells incubated with Raii-APC cells modified to express the FcγRII receptor by PD-1 antibodies with normal IgG1 Fc or modified Fc containing mutations to increase binding to FcγRII.

FIGS. 5A and 5B show that PD-1 antibody 314 containing modifications in the Fe domains to increase binding to FcγRII inhibited CD4 (FIG. 5A) and CD8 (FIG. 5B) T cell proliferation.

FIG. 6 shows that PD-1 antibody 3114 PD-1 antibody 314 containing modifications in the Fe domains to increase binding to FcγRII inhibited IFN-γ secretion from PBMCs compared to PD-1 antibodies with normal IgG1 Fe and controls (isotype antibody control and no antibody control).

### DETAILED DESCRIPTION

### Definitions

The terms “a” and “an” as used herein mean “one or more” and include the plural unless the context is inappropriate.

As used herein, the terms “about,” “approximately,” and “comparable to,” when used herein in reference to a value, refer to a value that is similar to the referenced value in the context of that referenced value. In general, those skilled in the art, familiar with the context, will appreciate the relevant degree of variance encompassed by “about,” “approximately,” and “comparable to” in that context. For example, in some embodiments, the terms “about,” “approximately,” and “comparable to” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, “agonist” or “agonistisc,” when used in reference to an antigen-binding site or a molecule containing an antigen-binding site, refers to that binding of the antigen-binding site or molecule to its target results in stimulation or activation of the target, or enhancement, increase, promotion, induction, or prolong of one or more functions or biological activity of the target.

As used herein, the term “antagonist,” “antagonistic,” “neutralizing,” or “blocking,” when used in reference to an antigen-binding site or a molecule containing an antigen-binding site, refers to that binding of the antigen-binding site or molecule to its target results in inhibition of at least some of the biological activity of the target.

As used herein, “antibody” refers to a polypeptide whose amino acid sequence includes immunoglobulins and fragments thereof which specifically bind to a designated antigen, or fragments thereof. Antibodies in accordance with the present invention may be of any type (e.g., IgA, IgD, IgE, IgG, or IgM) or subtype (e.g., IgA1, IgA2, IgG1, IgG2, IgG3, or IgG4). Those of ordinary skill in the art will appreciate that a characteristic sequence or portion of an antibody may include amino acids found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains, and may include sequence elements found in the same polypeptide chain or in different polypeptide chains.

As used herein, “antibody mimetic” refers to any molecule that is capable of mimicking an antibody's ability to bind an antigen, but which are not limited to antibody structures. Examples of antibody minetics include, but are not limited to, Affibodies, Affilins, Affimers, Afftins, Alphabodies, Anticalins, avimers, Centyrins, DARPins, Fynomers, monobodies, nanobody, and nanoCLAMPs.

An “antigen-binding fraigment” of an antibody comprises a portion of an intact antibody, which portion is still capable of antigen binding. In certain embodiments, papain digestion of antibodies produce two identical antigen-binding fragments, called “Fab” fragments, and a residual “Fc” fragment, a designation reflecting the ability to crystallize readily. The Fab fragment consists of an entire light chain along with the variable region domain of the heavy chain (Vi), and the first constant domain of one heavy chain (C_{H}1). Each Fab fragment is monovalent with respect to antigen binding, i.e., it has a single antigen-binding site. In certain embodiments, pepsin treatment of an antibody yields a single large F(ab′)₂ fragment which roughly corresponds to two disulfide linked Fab fragments having different antigen-binding activity and that is still capable of cross-linking antigen. Fab′ fragments differ from Fab fragments by having a few additional residues at the carboxy terminus of the C_{H}1 domain, including one or more cysteines from the antibody hinge region. Fab′-SH designates an Fab′ in which the cysteine residue(s) of the constant domains bear a free thiol group. F(ab′)₂ antibody fragments originally were produced as pairs of Fab′ fragments having hinge cysteines between them. Other chemical couplings of antibody fragments are also known.

As used herein, the term “antigen-binding site” refers to any molecule or any part of a molecule that is capable of antigen binding. In human antibodies, the antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus, the term “FR” refers to amino acid sequences which are naturally found between and adjacent to hypervariable regions in immunoglobulins. In a human antibodies, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site may by formed by a single antibody chain providing a “single domain antibody.” An antigen-binding site can also be or exist in an antibody mimetic.

The CDRs can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRHI1, CDRH2 and CDR13 denote the heavy chain CDRs, and CDRL1, CDRL2 and CDRL3 denote the light chain CDRs.

As used herein, “binding affinity” of a molecule of the disclosure (e.g., an antigent-binding site) to a given targets (e.g., PD-1, PD-L1, or PD-L2) can be determined by a multitude of methods known to those skilled in the art. Such methods include, but are not limited to, fluorescence titration, ELISA (enzyme-linked immunosorbent assay), calorimetric methods such as isothermal titration calorimetry (ITC), and surface plasmon resonance (SPR).

As used herein, “bispecific” refers to a molecule is able to specifically bind to at least two distinct targets. Typically, a bispecific molecule comprises two antigen-binding sites, each of which is specific for a different target. In some embodiments, a bispecific molecule is capable of binding two targets simultaneously.

As used herein, “detectable affinity” refers to the ability to bind to a given target with an affinity constant, typically measured by K_{D} or EC₅₀, of at most about 10⁻⁵ M or lower (a lower K_{D} or EC₅₀ value reflects better binding ability). Lower affinities that are no longer measurable with common methods such as ELISA (enzyme-linked immunosorbent assay) are of secondary importance.

As used herein, the term “epitope” is an antigenic determinant that interacts with a specific antigen binding site in the variable region of an antibody molecule, known as the paratope, and which is comprised of the six complementary-determining regions of the antibody. A single antigen may have more than one epitope. Epitopes may be conformational or linear. A conformational epitope is comprised of spatially juxtaposed amino acids from different segments of a linear polypeptide chain. A linear epitope is comprised of adjacent amino acid residues in a polypeptide chain.

An “Fe” fragment comprises the carboxy-terminal portions of both heavy chains held together by disulfides. The effector functions of antibodies are determined by sequences in the Fe region, the region which is also recognized by Fc receptors (FcR) found on certain types of cells.

A “fragment” with respect to an antigen (e.g., PD-1, PD-L1, or PD-L2) refers to N-terminally and/or C-terminally truncated or protein domains of the antigen. In some embodiments, a fragment of the antigen retains the capability of the full-length antigen to be recognized and/or bound by an antigen-binding site of the disclosure.

As used herein, “fucosylation” or “fucosylated” refers to the presence of fucose residues within the oligosaccharides attached to the peptide backbone of an antibody. Specifically, a fucosylated antibody comprises α(1,6)-linked fucose at the innermost N-acetylglucosamine (GilcNAc) residue in one or both of the N-linked oligosaccharides attached to the antibody Fc region. “Non-fucosylated,” “afucosylated,” or “fucose-deficient” antibody refers to a glycosylation antibody variant comprising an Fc region wherein a carbohydrate structure attached to the Fe region has reduced fucose or lacks fucose. In some embodiments, non-fucosylated or fucose-deficient antibodies have reduced fucose relative to the amount of fucose on the same antibody produced in a cell line. In some embodiments, an antibody with reduced fucose or lacking fucose has improved ADCC function.

The “degree of fucosylation” is the percentage of fucosylated oligosaccharides relative to all oligosaccharides, which may be identified by methods known in the art, e.g., in an N-glycosidase F treated antibody composition assessed by matrix-assisted laser desorption-ionization time-of-flight mass spectrometry (MALDI-TOF MS). In a composition of a “fully fucosylated antibody,” essentially all oligosaccharides comprise fucose residues, i.e., are fucosylated. In some embodiments, a composition of a fully fucosylated antibody has a degree of fucosylation of at least about 90%. In contrast, in a composition of a “fully non-fucosylated antibody,” essentially none of the oligosaccharides are fucosylated. In some embodiments, a composition of a fully non-fucosylated antibody has a degree of fucosylation of less than about 10%. In a composition of a “partially fucosylated antibody,” only part of the oligosaccharides comprise fucose. An individual antibody in such a composition may comprise fucose residues in none, one or both of the N-linked oligosaccharides in the Fe region, provided that the composition does not comprise essentially all individual antibodies that lack fucose residues in the N-linked oligosaccharides in the Fe region, nor essentially all individual antibodies that contain fucose residues in both of the N-linked oligosaccharides in the Fc region. In one embodiment, a composition of a partially fucosylated antibody has a degree of fucosylation of about 10% to about 80% (e.g., about 50% to about 80%, about 60% to about 80%, or about 70% to about 80%). Assays for measuring degree of fucosylation, as well as methods and cell lines for producing antibodies with altered, reduced, or eliminated fucosylation, are known in the art (see, e.g., WO 2023/044390 A1).

As used herein, the term “monoclonal antibody” refers to an antibody obtained from a population of substantially homogeneous antibodies, e.g., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, monoclonal antibodies may be made by a hybridoma method, or may be made by recombinant DNA methods (see, e.g., U.S. Pat. No. 4,816,567). “Monoclonal antibodies” may also be isolated from phage antibody libraries.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable salt” refers to any pharmaceutically acceptable salt (e.g., acid or base) of a compound described in the present application which, upon administration to a subject, is capable of providing a compound described in this application or an active metabolite or residue thereof. As is known to those of skill in the art, “salts” of the compounds described in the present application may be derived from inorganic or organic acids and bases.

Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds described in the present application and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds described in the present application compounded with a suitable cation such as Na⁺, NH₄⁺, and NW₄⁻ (wherein W is a C₁₋₄ alkyl group), and the like.

For therapeutic use, salts of the compounds described in the present application are contemplated as being pharmaceutically acceptable. However, salts of acids and bases that are non-pharmaceutically acceptable may also find use, for example, in the preparation or purification of a pharmaceutically acceptable compound.

As used herein, the phrase “reference level” generally refers to a level considered “normal” for comparison purposes, e.g., a level of an appropriate control. For example, in the context of antibody effector function enhancement, a “reference level” may refer to the level of effector function of an antibody comprising an wild-type constant (Fe) region. As another example, in the context of target binding, a “reference level” may refer to the level of binding affinity (e.g., defined by a K_{D} or EC₅₀ value) of a natural ligand of the target. A reference level may be determined contemporaneously or may be predetermined, e.g., known or deduced from past observations.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the phrases “therapeutically effective amount” and “effective amount” are used interchangeably and refer to an amount effective, at dosages and for periods of time necessary, to achieve a desired therapeutic result. A therapeutically effective amount may vary according to factors such as the type of disease (e.g., cancer), disease state, age, sex, and/or weight of the individual, and the ability of an immunoconjugate (or pharmaceutical composition thereof) to elicit a desired response in the individual. An effective amount may also be an amount for which any toxic or detrimental effects of the immunoconjugate or pharmaceutical composition thereof are outweighed by therapeutically beneficial effects.

As used herein, to “treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition (e.g., of a primary cancer and/or of a secondary metastases); delay or slowing the progress of the disease, disorder, or condition; amelioration or palliation of the disease, disorder, or condition; and remission (whether partial or total), whether detectable or undetectable.

### Antigen-Binding Sites

In one aspect, the present application provides an antigen-binding site that is capable of binding to a given antigen, e.g., an antigen on a cell involved in an immune disease. In some embodiments, the antigen-binding site capable of binding to an epitope within the antigen.

In some embodiments, the antigen-binding site is capable of binding to an antigen on a cell involved in an autoimmune, allergic, or inflammatory disease. In some embodiments, the cell is an immune cell, e.g., a T cell, a B cell, a mast cell, a macrophage, a natural killer (NK) cell, a dendritic cell (DC), a monocyte, a neutrophil, a fibroblast, or an epithelial cell. In some embodiments, the cell is an activated cell, e.g., an antigen-activated T cell or B cell. Examples of such antigens include, but are not limited to, PD-1, PD-L1, PD-L2, CTLA-4, a C-type lectin (CLEC), a Siglec, a tumor necrosis factor (TNF) receptor superfamily member, 4-1BB ligand (4-1BBL), X40 ligand (OX40L), CD40 ligand (CD40L), CD30 ligand (CD30L), CD70 ligand (CD70L), CD27 ligand (CD27L), TIM-3, LAG-3. BTLA, KLRG1, 2B4, CD244, CD19, C20, CD22, C28, (138, CD39, CD72, CD73, CD79A, CD79B, glycoprotein 130 (gp130), BCMN, BAFF receptor (BAFF-R), TACI, integrin alpha 4, integrin beta 7, FCGR2B, ICOS ligand, CD138, SLAMF7, a LILR family member, fibroblast activated protein alpha (FAP), DLK-1, CD26, TE-7, CD29, PDGRF alpha, TGF beta receptor, MAS516, CD13, or a combination thereof. In some embodiments, the antigen is PD-1, PD-L1, or PD-L2. In some embodiments, the antigen is PD-1.

In some embodiments, a provided antigen-binding sites is present as an antibody mimetic, e.g., an Affibody, an Affilin, an Affimer, an afftin, an Alphabody, an Anticalim, an avimer, a Centyrin, a DARPin, a Fynomer, a monobody, nanobody, or a nanoCLAMP.

In some embodiments, a provided antigen-binding sites comprises a heavy chain variable domain and/or a light chain variable domain. In some embodiments, the antigen-binding sites comprise a heavy chain variable domain comprising CDR-H1, CDR-H2, and CDR-H3, and/or a light chain variable domain comprising CDR-L1, CDR-L2, and CDR-H3. In some embodiments, the antigen-binding site is present as a single-chain fragment variable (scFv).

In some embodiments, provided antigen-binding sites are capable of binding PD-1. In some embodiments, the antigen-binding site is present as an antibody. In some embodiments, provided are antigen-binding sites that are variants of an antigen-binding site capable of binding PD-1 and present as an antibody described herein, in that such antigen-binding sites comprise an amino acid sequence that is at least 85%, at least 87.5%, at least 90%, at least 91%, at least 92%, at least 93%, at least 94%, at least 95%, at least 96%, at least 97%, at least 98%, or at least 99% identical to an amino acid sequence comprised in said antigen-binding site capable of binding PD-1 and present as an antibody described herein.

In some embodiments, provided antigen-binding sites capable of binding PD-1 comprise a heavy chain variable domain (VH) comprising a CDR-H1, CDR-H2, and CDR-H3, and/or a light chain variable domain (VL) comprising a CDR-L1, CDR-L2, and CDR-L3.

In some embodiments, provided antigen-binding sites capable of binding PD-1 comprise a VH and a VL, wherein the VH comprises a CDR-H1, CDR-H2, and CDR-H3, and the VL comprises a CDR-L1, CDR-L2, and CDR-L3, wherein the CDR-H1, CDR-H2, CDR-H3, CDR-L1, CDR-L2, and CDR-L3 are each independently selected from those of a VH or VL described in Table 5. CDRs are determined under Kabat, AbM, Chothia, or any other CDR determination method known in the art, of the VH and VL. In certain embodiments, the antigen-binding site comprises the CDR-H1, CDR-H2, CDR-H3, CDR-L1, CDR-L2, and CDR-L3 each independently selected from those described in Table 5.

In some embodiments, provided are antigen-binding sites that are variants of the antigen-binding sites capable of binding PD-1 described herein, in that such antigen-binding sites have CDR sequences that differ by no more than two amino acid residues (e.g., two or one amino acid residue(s)) per CDR from a CDR sequence described in Table 5. In some embodiments, provided are an antigen-binding sites that are variants of the antigen-binding sites capable of binding PD-1 described herein, in that such antigen-binding sites have a set of six CDRs whose sequences collectively differ by no more than two amino acid residues (e.g., two or one amino acid residues) from a set of CDR-H1, CDR-H2, CDR-H3, CDR-L1, CDR-L2, and CDR-L3 selected from Table 5. In some embodiments, provided are an antigen-binding sites that are variants of the antigen-binding sites capable of binding PD-1 described herein, in that such antigen-binding sites have a set of six CDRs whose sequences collectively differ by no more than two amino acid residues (e.g., two or one amino acid residues) from those of an anti-PD-1 having a set of VH and VL selected from Table 5.

In some embodiments, provided antigen-binding sites capable of binding PD-1 comprise a VH sequence as described in Table 5 and a VL sequence as described in Table 5. In some embodiments, provided are antigen-binding sites that are variants of the antigen-binding site capable of binding PD-1 described herein, such as an anti-PD-1 having a set of VH and VL selected from Table 5, in that such antigen-binding site have (1) a VH comprising an amino acid sequence that is at least 859%, at least 87.5%, at least 90%, at least 91%, at least 92%, at least 93%, at least 94%, at least 95%, at least 96%, at least 97%, at least 98%, or at least 99% identical to the amino acid sequence of a VH described in Table 5; and (2) a light chain domain comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of a NL described in Table 5.

In some embodiments, provided antigen-binding sites capable of binding PD-1 comprise a heavy chain sequence as described in Table 5, and a light chain sequence as described in Table 5. In some embodiments, provided are antigen-binding sites that are variants of the antigen-binding site capable of binding PD-1 described herein, such as an anti-PD-1 having a set of heavy chain and light chain selected from Table 5, in that such antigen-binding site have (1) a heavy chain comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of a heavy chain described in Table 5; and (2) a light chain comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of a light chain described in Table 5.

In certain embodiments, an antigen-binding site described in the present application is derived from 1-17, For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 1, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ LD NO: 5. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 1 and 5, respectively. In certain embodiments, the VH comprises CDR-H1, CDR-H2, and CDR-H3, comprising the amino acid sequences of SEQ ID NOs: 2, 3, and 4, respectively. In certain embodiments, the VL comprises CDR-L1, CDR-L2, and CDR-L3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ LD NOs: 2, 3, and 4, respectively; and (b) a VL that comprises CDR1, CDR2, and CDR3 comprising the amino acid sequences of SEQ ID NOs: 6, 7, and 8, respectively.

In certain embodiments, an antigen-binding site described in the present application is derived from 1-28. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 13. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 9 and 13, respectively. In certain embodiments, the VII comprises CDR-H1, CDR-H2, and CDR-H3, comprising the amino acid sequences of SEQ ID NOs: 10, 11, and 12, respectively. In certain embodiments, the VL comprises CDR-L1, CDR-L2, and CDR-L3 comprising the amino acid sequences of SEQ ID NOs: 14, 15, and 16, respectively. In certain embodiments, the antigen-binding site comprises (a) a VH that comprises CDR1, CDR2, and CDR3 comprising the amino acid sequences of SEQ ID NOs: 10, 11, and 12, respectively; and (b) a VL that comprises CDR1, CDR2, and CDR3 comprising the amino acid sequences of SEQ ID NOS: 14, 15, and 16, respectively.

In certain embodiments, an antigen-binding site described in the present application is derived from 1-33. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 17, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 21. In certain embodiments, the antigen-binding site comprises the CDR-H11, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 17 and 21, respectively. In certain embodiments, the VH comprises CDR-H1, CDR-H2, and CDR-H3, comprising the amino acid sequences of SEQ ID NOs: 18, 19, and 20, respectively. In certain embodiments, the VL comprises CDR-L1, CDR-L2, and CDR-L3 comprising the amino acid sequences of SEQ ID NOs: 22, 23, and 24, respectively. In certain embodiments, the antigen-binding site comprises (a) a VH that comprises CDR1, CDR2, and CDR3 comprising the amino acid sequences of SEQ ID NOs: 18, 19, and 20, respectively; and (b) a VL that comprises CDR1, CDR2, and CDR3 comprising the amino acid sequences of SEQ ID NOs: 22, 23, and 24, respectively.

In certain embodiments, an antigen-binding site described in the present application is derived from 1-35. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 25, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 29. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 25 and 29, respectively. In certain embodiments, the VH comprises CDR-H1, CDR-H2, and CDR-H3, comprising the amino acid sequences of SEQ ID NOs: 26, 27, and 28, respectively. In certain embodiments, the VL comprises CDR-L1, CDR-L2, and CDR-L3 comprising the amino acid sequences of SEQ ID NOs: 30, 31, and 32, respectively. In certain embodiments, the antigen-binding site comprises (a) a VH that comprises CDR1, CDR2, and CDR3 comprising the amino acid sequences of SEQ ID NOs: 26, 27, and 28, respectively; and (b) a VL that comprises (CDR1, CDR2, and CDR3 comprising the amino acid sequences of SEQ ID NOs: 30, 31, and 32, respectively.

In certain embodiments, an antigen-binding site described in the present application is derived from murine 3H4. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 33, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 37. In certain embodiments, the antigen-binding site comprises the CDR-H1-1, CDR-2, CDR-H3, CDR-L1, CDR-L2, and CDR-L3, determined under Kabat, AbM, Chothia, or any other CDR determination method known in the art, of the VH and VL sequences of SEQ ID NOs: 33 and 37, respectively. In certain embodiments, the VH comprises CDR-H1, CDR-H2, and CDR-H3, comprising the amino acid sequences of SEQ ID NOs: 34, 35, and 36, respectively. In certain embodiments, the VL comprises CDR-L1, CDR-L2, and CDR-L3 comprising the amino acid sequences of SEQ ID NOs: 38, 39, and 40, respectively. In certain embodiments, the antigen-binding site comprises (a) a VH that comprises CDR1, CDR2, and CDR3 comprising the amino acid sequences of SEQ ID NOs: 34, 35, and 36, respectively; and (b) a VL that comprises CDR1, CDR2, and CDR3 comprising the amino acid sequences of SEQ ID NOs: 38, 39, and 40, respectively.

In certain embodiments, an antigen-binding site described in the present application is derived from humanized 31H4. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 42 or 45, and/or a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 47 or 49. In certain embodiments, the VH comprises an amino acid sequence of SEQ ID NO: 42. In certain embodiments, the VH comprises an amino acid sequence of SEQ ID NO: 44. In certain embodiments, the VL comprises an amino acid sequence of SEQ ID NO: 47. In certain embodiments, the VL comprises an amino acid sequence of SEQ ID NO: 49. In certain embodiments, the antigen-binding site comprises the CDR-H1, CDR-H2, and CDR-H3, determined under Kabat, AbM, Chothia, or any other CDR determination method known in the art, of the VH sequence of SEQ FD NO: 42 or 45; and/or the CDR-L1, CDR-L2, and CDR-L3, determined under Kabat, AbM, Chothia, or any other CDR determination method known in the art, of the VL sequence of SEQ ID NO: 47 or 49. In certain embodiments, the antigen-binding site comprises (a) a VH that comprises CDR-H1, CDR-H2, and CDR-H3 comprising the amino acid sequences of SEQ ID NOs: 34, 43, and 36, respectively; and (b) a VL that comprises CDR-L1, CDR-L2, and CDR-L3 comprising the amino acid sequences of SEQ ID NOs: 38, 39, and 40, respectively.

In certain embodiments, an antigen-binding site described in the present application is derived from murine 2.3A9. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 50, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 54. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VII and VL sequences of SEQ ID NOs: 50 and 54, respectively. In certain embodiments, the VH comprises CDR-11, CDR-2, and CDR-H3, comprising the amino acid sequences of SEQ ID NOs: 51, 52, and 53, respectively. In certain embodiments, the VL comprises CDR-L1, CDR-L2, and CDR-L3 comprising the amino acid sequences of SEQ ID NOs: 55, 56, and 57, respectively. In certain embodiments, the antigen-binding site comprises (a) a VH that comprises CDR1, CDR2, and CDR3 comprising the amino acid sequences of SEQ ID NOs: 51, 52, and 53, respectively; and (b) a VL that comprises CDR1, CDR2, and CDR3 comprising the amino acid sequences of SEQ ID NOs: 55, 56, and 57, respectively.

In certain embodiments, an antigen-binding site described in the present application is derived from humanized 23A9. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 59, 62, 64, or 66, and/or a V L that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 68. In certain embodiments, the VH comprises an amino acid sequence of SEQ ID NO: 59. In certain embodiments, the VH comprises an amino acid sequence of SEQ ID NO: 62. In certain embodiments, the VH comprises an amino acid sequence of SEQ ID NO: 64. In certain embodiments, the VH comprises an amino acid sequence of SEQ ID NO: 66. In certain embodiments, the VL comprises an amino acid sequence of SEQ ID NO: 68 In certain embodiments, the antigen-binding site comprises the CDR-H1, CDR-H2, and CDR-H3, determined under Kabat, AbM, Chothia, or any other CDR determination method known in the art, of the VH sequence of SEQ ID NO: 59, 62, 64, or 66; and/or the CDR-L1, CDR-L2, and CDR-L3, determined under Kabat, AbM, Chothia, or any other CDR determination method known in the art, of the VL sequence of SEQ ID NO: 68, In certain embodiments, the antigen-binding site comprises (a) a VH that comprises CDR-H1, CDR-H2, and CDR-H3 comprising the amino acid sequences of SEQ ID NOs: 51, 60, and 53, respectively; and (b) a VL that comprises CDR-L1, CDR-L2, and CDR-L3 comprising the amino acid sequences of SEQ ID NOs: 55, 56, and 57, respectively.

In certain embodiments, an antigen-binding site described in the present application is derived from murine 20B3.1. For example, in certain embodiments, an antigen-binding site described in the present application comprises a VII that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 69, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 73. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and L sequences of SEQ ID NOs: 69 and 73, respectively. In certain embodiments, the VII comprises CDR-H1, CDR-H2, and CDR-H3, comprising the amino acid sequences of SEQ ID NOs: 70, 71, and 72, respectively. In certain embodiments, the VL comprises CDR-L1, CDR-L2, and CDR-L3 comprising the amino acid sequences of SEQ ID NOs: 74, 75, and 76, respectively. In certain embodiments, the antigen-binding site comprises (a) a VH that comprises CDR1, CDR2, and CDR3 comprising the amino acid sequences of SEQ ID NOs: 70, 71, and 72, respectively; and (b) a VL that comprises CDR1, CDR2, and CDR3 comprising the amino acid sequences of SEQ ID NOs: 74, 75, and 76, respectively.

In certain embodiments, an antigen-binding site described in the present application is derived from humanized 20B3.1. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 78, and/or a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 81 or 83. In certain embodiments, the VH comprises an amino acid sequence of SEQ ID NO: 78. In certain embodiments, the VL comprises an amino acid sequence of SEQ ID NO: 81. In certain embodiments, the VL comprises an amino acid sequence of SEQ ID NO: 83. In certain embodiments, the antigen-binding site comprises the CDR-H1, CDR-H2, and CDR-H3, determined under Kabat, AbM, Chothia, or any other CDR determination method known in the art, of the VH sequence of SEQ ID NO: 78; and/or the CDR-L1, CDR-L2, and CDR-L3, determined under Kabat, AbM, Chothia, or any other CDR determination method known in the art, of the VL sequence of SEQ ID NO: 81 or 83. In certain embodiments, the antigen-binding site comprises (a) a VH that comprises CDR-H1, CDR-H2, and CDR-H3 comprising the amino acid sequences of SEQ ID NOs: 70, 79, and 72, respectively; and (b) a VL that comprises CDR-L1, CDR-L2, and CDR-L3 comprising the amino acid sequences of SEQ ID NOs: 74, 75, and 76, respectively.

In certain embodiments, an antigen-binding site described in the present application is derived from murine IBS. For example, in certain embodiments, an antigen-binding site described in the present application comprises a Vi that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 84, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 88. In certain embodiments, the antigen-binding site comprises the CDR-1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 84 and 88, respectively. In certain embodiments, the VII comprises CDR-1-1, CDR-H2, and CDR-H3, comprising the amino acid sequences of SEQ ID NOs: 85, 86, and 87, respectively. In certain embodiments, the VL, comprises CDR-L1, CDR-L2, and CDR-L3 comprising the amino acid sequences of SEQ ID NOs: 89, 90, and 91, respectively. In certain embodiments, the antigen-binding site comprises (a) a VH that comprises CDR1, CDR2, and CDR3 comprising the amino acid sequences of SEQ ID NOs: 85, 86, and 87, respectively; and (b) a VL that comprises CDR1, CDR2, and CDR3 comprising the amino acid sequences of SEQ ID NOs: 89, 90, and 91, respectively.

In certain embodiments, an antigen-binding site described in the present application is derived from humanized 1B8. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 93, and/or a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 96 or 98. In certain embodiments, the VH comprises an amino acid sequence of SEQ ID NO: 93. In certain embodiments, the VL comprises an amino acid sequence of SEQ ID NO: 96. In certain embodiments, the VL comprises an amino acid sequence of SEQ ID NO: 98. In certain embodiments, the antigen-binding site comprises the CDR-H11, CDR-H2, and CDR-H3, determined under Kabat, AbM, Chothia, or any other CDR determination method known in the art, of the VH sequence of SEQ ID NO: 93; and/or the CDR-L1, CDR-L2, and CDR-L3, determined under Kabat, AbM, Chothia, or any other CDR determination method known in the art, of the VL sequence of SEQ ID NO: 96 or 98. In certain embodiments, the antigen-binding site comprises (a) a VH that comprises CDR-H1, CDR-H2, and CDR-H3 comprising the amino acid sequences of SEQ ID NOs: 86, 94, and 87, respectively; and (b) a VL that comprises CDR-L1, CDR-L2, and CDR-L3 comprising the amino acid sequences of SEQ ID NOs 89, 90, and 91, respectively.

In each of the foregoing embodiments, it is contemplated herein that the YR and/or VL sequences that bind PD-1 may contain amino acid alterations (e.g., at least 1, 2, 3, 4, 5, or amino acid substitutions, deletions, or additions) in the framework regions of the VH and/or VL without affecting their ability to bind PD-IL For example, it is contemplated herein that VH and VL sequences that bind PD-1 may contain cysteine heterodimerization mutations, facilitating formation of a disulfide bridge between the VH and VL to form an scFv.

In some embodiments, antigen-binding sites disclosed herein bind human PD-1 or the extracellular region thereof. In some embodiments, antigen-binding sites disclosed herein bind PD-1 presented on the surface of a membrane (e.g., plasma membrane of a cell).

In some embodiments, antigen-binding sites disclosed herein bind cynomolgus PD-1. In some embodiments, antigen-binding sites disclosed herein bind cynomolgus PD-1 at a comparable affinity to that of binding human PD-1.

In some embodiments, antigen-binding sites disclosed herein does not significantly bind other PD-1 family members. In some embodiments, antigen-binding sites disclosed herein does not significantly bind ICOS, CD28, or CTLA-4.

In some embodiments, antigen-binding sites disclosed herein are capable of inhibiting binding of PD-L1 to PD-1. In some embodiments, antigen-binding sites disclosed herein are capable of agonizing PD-1.

In some embodiments, antigen-binding sites disclosed herein are capable of reducing IL-2 secretion. In some embodiments, antigen-binding sites disclosed herein are capable of inhibiting (partially or fully) T cell activation. In some embodiments, antigen-binding sites disclosed herein are capable of inhibiting (partially or fully) T cell proliferation (e.g., CD4+ and/or CD8+ T cells).

In some embodiments, antigen-binding sites disclosed herein are capable of reducing inflammatory cytokine secretion. In some embodiments, antigen-binding sites disclosed herein are capable of reducing IFN-γ secretion.

### Molecules Containing Antigen-Binding Sites

Also provided herein are molecules containing disclosed antigen-binding sites. Such molecules may be, but are not limited to, antibodies or antigen-binding fragments thereof, antibody fragments, nanobodies, antibody mimetics, etc. In some embodiments, a molecule containing disclosed antigen-binding sites is a therapeutic agent, i.e., the molecule confers a therapeutic benefit. Accordingly, in some embodiments, provided are therapeutic agents containing disclosed antigen-binding sites. In some embodiments, the therapeutic agent is capable of inducing complement dependent cytotoxicity (CDC). In some embodiments, the therapeutic agent is capable of inducing antibody-dependent cell-mediated cytotoxicity (ADCC). In some embodiments, therapeutic agent is capable of inducing antibody-dependent cellular phagocytosis (ADCP). In some embodiments, the therapeutic agent is capable of depleting an immune cell (e.g., a T cell, a B cell, a mast cell, a macrophage, a NK cell, a DC, a monocyte, a neutrophil, a fibroblast, or an epithelial cell).

In some embodiments, the therapeutic agents are antibodies or antigen-binding fragments thereof. In some embodiments, the term “antigen-binding fragment” includes, but not limited to, an Fab, an scFab (single-chain Fab), an F(ab′)₂, a Fab′, a single-chain Fv (scFv), an Fv fragment, or a diabody (a dimer of scFv). In some embodiments, the antibodies are monoclonal antibodies. In some embodiments, the antibodies are humanized antibodies. In some embodiments, the antibodies are human antibodies. In some embodiments, the antibodies or antigen-binding fragments thereof are multispecific, e.g., bispecific. In some embodiments, the antibodies are non-fucosylated (afucosylated). In some embodiments, the antibodies are at most about 90%, at most about 80%, at most about 70%, at most about 60%, at most about 50%, at most about 40%, at most about 30%, at most about 25%, at most about 20%, at most about 15%, at most about 10%, at most about 5%, at most about 4%, at most about 3%, at most about 2%, at most about 1% fucosylated. In some embodiments, the antibodies are afucosylated. In some embodiments, the antibodies are at least about 30%, at least about 40%, at least about 50%, at least about 60%, at least about 70%, at least about 75%, at least about 80%, at least about 85%, at least about 90%, at least about 95%, at least about 96%, at least about 97%, at least about 98%, or at least about 99% afucosylated.

In some embodiments, the antibodies or antigen-binding fragments thereof are capable of binding PD-1, PD-L1, PD-L2, CTLA-4, a C-type lectin (CLEC), a Siglec, a tumor necrosis factor (TNF) receptor superfamily member, 4-1BB ligand (4-1BBL), OX40 ligand (OX40L), CD40 ligand (CD40L), CD30 ligand (CD30L), CD70 ligand (CD70L), CD27 ligand (CD27L), TIM-3, LAG-3, BTLA, KLRG1, 2B4, CD244, CD19, CD20, CD22, CD28, CD38, CD39, CD72, CD73, CD79A, CD79B, glycoprotein 130 (gp130), BCMA, BAFF receptor (BAFF-R), TACI, integrin alpha 4, integrin beta 7, FCGR2B, ICOS ligand, CD138, SLAMF7, a LILR family member, fibroblast activated protein alpha (FAP), DLK-1, CD26, TE-7, CD29, PDGRF alpha, TGF beta receptor, MAS516, CD13, or a combination thereof. In some embodiments, the antibodies or antigen-binding fragments thereof are capable of binding PD-1, PD-L1, or PD-L2. In some embodiments, the antibodies or antigen-binding fragments thereof are capable of binding PD-L In some embodiments, the antibodies or antigen-binding fragments thereof are capable of agonizing PD-1.

In some embodiments, provided are therapeutic agents comprising two or more antigen-binding sites. In some embodiments, the therapeutic agents are bispecific.

In some embodiments, the therapeutic agent comprises a first antigen-binding site capable of binding a marker for activated T cell, activated B cell, activated fibroblast, or activated epithelial cell. In some embodiments, the first antigen-binding site is capable of binding PD-1, PD-L1, PD-L2, CTLA-4, BTLA-4, or KLRG1. In some embodiments, the first antigen-binding site is capable of binding PD-1, PD-L1, PD-L2, BCMA, or CD38. In some embodiments, the first antigen-binding site is capable of binding FAP or PDGRF alpha.

In some embodiments, the therapeutic agent comprises a second antigen-binding site is capable of binding a marker for activated T cell, activated B cell, activated fibroblast, or activated epithelial cell. In some embodiments, the marker is for the same type of activated cell for which the marker that the first antigen-binding site binds. In some embodiments, the marker is for a different type of activated cell for which the marker that the first antigen-binding site binds.

In some embodiments, the markers for activated T cell include, but are not limited to, TIM-3 and LAG-3. In some embodiments, the markers for activated B cell include, but are not limited to, CD19, CD20, BAFF-R, and TACI In some embodiments, the markers for activated fibroblast include, but are not limited to, FAP, DLK-1, CD26, TE-7, CD29, PDGRF alpha, TGF beta receptor, MAS516, and CD13.

Various formats and uses of bispecific molecules are known in the art. Bispecific molecules according to the present invention are not limited to any particular bispecific format or method of producing it.

In some embodiments, the bispecific molecule may be a bispecific antibody or a fragment or derivative thereof including, but not limited to, (i) a single antibody that has two arms, each comprising a different antigen-binding site; (ii) a bispecific scFv, e.g., via two scFvs linked by a peptide linker; (iii) a dual-variable-domain antibody (DVD-Ig), where each light chain and heavy chain contains two variable domains in tandem through a short peptide linkage; (iv) a bispecific (Fab′)₂ fragment; (v) a diabody; (vi) a tandem diabody (TandAb), a fusion of two diabodies; and (vii) a “dock-and-lock (DNL)-Fab3, a trivalent bispecific binding protein consisting of two identical Fab fragments linked to a different Fab fragment; (viii) IgG-like molecules with engineered Fe to force heterodimerization; (ix) recombinant IgG-like dual targeting molecules, wherein the two sides of the molecule each contain the Fab fragment or part of the Fab fragment of at least two different antibodies, (x) IgG fusion molecules, wherein full length IgG antibodies are fused to extra Fab fragment or parts of Fab fragment; (xi) Fe fusion molecules; (v) Fab fusion molecules; and (vi) scFv- and diabody-based and heavy chain antibodies (e.g., domain antibodies, nanobodies) wherein different scFvs, diabodies, or heavy-chain antibodies (e.g., domain antibodies, nanobodies) are fused to each other or to another protein or carrier molecule fused to heavy-chain constant-domains, Fe-regions or parts thereof.

Examples of IgG-like molecules with engineered Fc include, but are not limited, to the triomab, knobs-into-holes (kih) molecules (e.g., kih IgG with common light chain), CrossMAbs, orthoFab IgG molecules electrostatically-matched molecules, the LUZ-Y molecules, DIG-body and PIG-body molecules, the Strand Exchange Engineered Domain body (SEEDbody) molecules, the Biclonics molecules, FcΔAdp molecules, bispecific IgG1 and IgG2 molecules, Azymetric scaffold molecules, and the DuoBody molecules. Examples of recombinant IgG-like dual targeting molecules include, but are not limited, to Dual Targeting (DT)-Ig molecules, two-in-one antibody, mAb², and Zybody. Examples of IgG fusion molecules include, but are not limited to, DVD-Ig molecules and IgG-scFv. Examples of Fe fusion molecules include, but are not limited to, scFv/Fc fusions, SCORPION molecules, and Fc-DART molecules. Examples of Fab fusion bispecific antibodies include, but are not limited to, F(ab)₂ molecules, DNL molecules, and Fab-Fv molecules. Examples of scFv- and diabody-based and domain antibodies include, but are not limited, to bispecific T cell engager (BiTE) molecules, tandem diabody molecules (TandAb), dual-affinity retargeting technology (DART) molecules, single-chain diabody molecules, TCR-like antibodies (AIT, ReceptorLogics), human serum albumin scfv fusion, COMBODY molecules, dual targeting nanobodies, and dual targeting heavy chain only domain antibodies.

In some embodiments, molecules containing disclosed antigen-binding sites further comprise antibody constant regions or fragments or variants thereof. In some embodiments, the antibody constant region may be, e.g., a heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In certain embodiments, the antibody constant region or fragment or variant thereof has an amino acid sequence at least 90% (e.g., 90%, 91%, 92%, 93%, 94%, 95%, 96%, 97%, 98%, 99%, or 100%) identical to a heavy chain constant region of, e.g., the heavy chain constant region of IgG1, IgG2, IgG3, IgG4, IgM, IgA1, IgA2, IgD, or IgE, preferably, e.g., the heavy chain constant region of human IgG1, IgG2, IgG3, or IgG4. In certain embodiments, the antibody constant region may be a light chain constant region chosen from, e.g., the (e.g., human) light chain constant regions of kappa or lambda. The constant region can be altered, e.g., mutated, to modify the properties of the antibody (e.g., to increase or decrease one or more of Fc receptor binding, antibody glycosylation, the number of cysteine residues, effector cell function, and/or complement function). In one embodiment, the antibody has effector function and/or can fix complement. In other embodiments, the antibody does not recruit effector cells or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In some embodiments, the constant region comprises one or more mutation(s) that enhance effector function.

In some embodiments, the constant region comprises one or more mutation(s) that enhance binding to FcγRII (CD32). For example, in certain embodiments, the constant region comprises a heavy chain constant region of IgG1, comprising one or more of the following mutations, numbering based on EU index, (a) E233D; (b) G236D; (c) G237D; (d) P2381D; (e) S239D; (f) S2671E (g) HI268D; (h) P271G; (i) L328Y, L328F, or L328E; (j) A330R; (k) I332E; (l) G236D and H268D; (in) S239D and H268D; (n) S239D. H268D, L328Y, and I332E; (o) P238) and L328E; (p) G237D, P271G, and A330R; (q) G237D, H268D, P271G, and A330R; (r) S267E and L328F; (s) S239D and S267E; (t) G2361) and S276E; (u) E233D, G237D, H268D, P271G, and A330R, or a combination thereof. In certain embodiments, the constant region comprises a heavy chain constant region of IgG1 comprising the mutations of S239D and H268D, numbering based on EU index. In certain embodiments, the constant region comprises a heavy chain constant region of IgG1 comprising the mutations of E233D, G237D, H268D, P271G, and A330R, numbering based on EU index.

In certain embodiments, the constant region comprises a heavy chain constant region of IgG1, comprising one or more of the following mutations, numbering based on EU index, F243L, R292P, Y300L, V3051, and/or P396L; (b) S239D and/or I332E; (c) S239D, I332E, and/or A330L; (d) S298A, E333A, and/or K334A; (e) G236A, S239D, and/or I332E; (f) K326W and/or E333S; (g) S267E, HI268F, and/or S324T; or (h) E345R, E430G, and/or S440Y.

In some embodiments, the constant region comprises one or more mutation(s) that reduce effector function. For example, in certain embodiments, the constant region comprises a heavy chain constant region of IgG1, comprising one or more of the following mutations, numbering based on EU index, (a) L234A and/or L235A; (b) A327G, A330S, and/or P331 S; (c) E233P, L234V, I235A, and/or G236del; (d) E233P, L234V, and/or L235A; (e) E233P, L234V, L235A, G236del, A327G, A330S, and/or P331S; (f) E233P, L234V, L235A, A327G, A330S, and/orP331 S; (g) N297A; (h) N297G; (i) N297Q; (i) L242C, N297C, and/or K334C; (k) A287C, N²97G, and/or L306C; (1) R292C, N297G, and/or V302C; (in) N297G, V323C, and/or I332C; (n) V259C, N297G, and/or L306C; (o) L234F, L235Q, K322Q, M252Y, 8254T, and/or T256E; (p) L234A, L235AN, and/or P329G; or (q) L234A, L235Q, and K322Q. In certain embodiments, the constant region comprises a heavy chain constant region of IgG2, comprising one or more of the followirig mutations, numbering based on EU index, (a) A330S and/or P331S; (b) V234A, G237A, P238S, H268A, V309L, A330S, and/or P331S; or (c) V234A, G237A, H268Q, V309L A330S, P331 S, C232S, C233S, S267E, L328F, M252Y, 82541, and/or T256E. In certain embodiments, the constant region comprises a heavy chain constant region of IgG4, comprising one or more of the following mutations, numbering based on EU index, (a) E233P, F234V, L235A, and/or G236del; (b) E233P, F234V, and/or L235A; (c) S228P and/or L235E; or (d) S228P and/or L235A.

In some embodiments, the antigen-binding site is linked to the antibody constant region or fragment or variant thereof. In certain embodiments, the antigen-binding site is linked to an IgG constant region including hinge, CH₂ and CH₃ domains with or without a CH₁ domain.

In some embodiments, molecules containing disclosed antigen-binding sites further comprises a Fc region that is non-fucosylated. In some embodiments, the Fe region is non-fucosylated IgG1 Fc region.

In some embodiments, the bispecific molecule may be a fusion protein containing one or more antibody mimetics. In some embodiments, the bispecific molecule may be a fusion protein containing one or more antibody mimetics and one or more antibodies or antigen-binding fragments thereof.

### Amino Acid Sequence Modifications

Amino acid sequence modification(s) of the antigen-binding sites and molecules containing antigen-binding sites (e.g., antibodies or antigen-binding fragments thereof) disclosed herein are contemplated. For example, it may be desirable to improve the binding affinity and/or other biological properties of the antibodies or antigen-binding fragments. Amino acid sequence variants can be prepared, e.g., by introducing appropriate nucleotide changes into a nucleic acid sequence encoding the antigen-binding site or molecule containing antigen-binding site, or by peptide synthesis. Such modifications can include, for example, deletions from, and/or insertions into and/or substitutions of, residues within the amino acid sequences. Any combination of deletion, insertion, and substitution can be made, provided that the antigen-binding site or molecule containing antigen-binding site retains the desired characteristics and/or functions. In some embodiments, amino acid changes are introduced to alter post-translational processes, such as changing the number or position of glycosylation sites.

A useful method for identification of certain residues or regions that are preferred locations for mutagenesis is called “alanine scanning mutagenesis.” In this method, a residue or group of target residues are identified (e.g., charged residues such as Arg, Asp, His, Lys, and Glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis may be conducted at the target codon or region, and the expressed variants may be screened for a desired activity.

Examples of amino acid sequence insertions include, but are not limited to, amino- and/or carboxyl-terminal fusions ranging in length from one residue to polypeptides containing a hundred or more residues, as well as intrasequence insertions of single or multiple amino acid residues. An example of a terminal insertion includes, but are not limited to, N-terminal methionyl residues.

In some embodiments, the antigen-binding site or molecule containing antigen-binding site is fused at one terminus to another polypeptide, e.g., a cytotoxic polypeptide, an enzyme, or a polypeptide which increases the serum half-life of the antibody or antigen-binding fragment.

Another type of variant is an amino acid substitution variant. These variants have at least one amino acid residue in the amino acid sequence of the molecule replaced by a different residue. Sites of greatest interest for substitutional mutagenesis are typically the hypervariable regions, but framework region alterations are also contemplated. Examples of conservative substitutions are shown in Table 1 under the heading of “preferred substitutions.” More substantial changes, under the heading “exemplary substitutions” in Table 1, or as further described below in reference to amino acid classes, may be introduced and the resulting antibodies or antigen-binding fragments screened.

TABLE 1
Exemplary Amino acid substitutions

| Original | | Preferred |
|---|---|---|
| Residue | Exemplary Substitutions | Substitutions |
| Ala (A) | Val, Leu, Ile | Val |
| Arg (R) | Lys, Gln, Asn | Lys |
| Asn (N) | Gln, His, Asp, Lys, Arg | Gln |
| Asp (D) | Glu. Asn | Glu |
| Cys (C) | Ser, Ala | Ser |
| Gln (Q) | Asn, Glu | Asn |
| Glu (E) | Asp, Gln | Asp |
| Gly (G) | Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, Gln, Asn | Arg |
| Met (M) | Leu Phe, Ile | Leu |
| Phe (F) | Leu, Val, Ile, Ala, Tyr | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, Norleucine | Leu |

Substantial modifications in the biological properties of the antibody may be accomplished by selecting substitutions that differ significantly in their effect on maintaining
(a) the structure of the polypeptide backbone in the area of the substitution, for example, as a sheet or helical conformation, (b) the charge or hydrophobicity of the molecule at the target site, or (c) the bulk of the side chain. Naturally occurring residues are typically divided into groups based on common side-chain properties;
   (1) hydrophobic: Norleucine, Met, Ala, Val, Leu, Ile;
   (2) neutral hydrophilic: Cys, Ser, Thr;
   (3) acidic: Asp, Glu;
   (4) basic: Asn, Gln, His, Lys, Arg;
   (5) residues that influence chain orientation: Gily, Pro; and
   (6) aromatic: Trp, Tyr, Phe.

Non-conservative substitutions can entail exchanging a member of one of these classes for another class.

Additionally or alternatively, cysteine residues not involved in maintaining the proper conformation of the antibody or antigen-binding fragment may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

In some embodiments, a substitutional variant comprises a substitution within one or more hypervariable region residues of a parent antibody (e.g., a human antibody). Generally, the resulting variant(s) having improved biological properties relative to the parent antibody from which they are generated are selected for further development.

A method for generating such substitutional variants involves affinity maturation using phage display. In an example of such a method, several hypervariable region sites (e.g., 6-7 sites) are mutated to generate all possible amino substitutions at each site. Antibody variants thus generated are displayed in a monovalent fashion, e.g., from filamentous phage particles as fusions to the gene III product of M13 packaged within each particle. The phage-displayed variants are then screened for their biological activity (e.g., binding affinity).

To identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody or antigen-binding fragment and the antigen. Such contact residues and neighboring residues are candidates for substitution according to the techniques elaborated herein. Once such variants are generated, the panel of variants is subjected to screening, and antibodies with superior properties in one or more relevant assays may be selected for further development.

In some embodiments, the original glycosylation pattern of a parent antibody is altered. Such alteration(s) may comprise deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are recognition sequences for enzymatic attachment of the carbohydrate moiety to an asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody may be accomplished by altering the antibody or antigen-binding fragment's amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of antibodies or antigen-binding fragments may be prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody or antigen-binding fragment thereof.

In some embodiments, a modification that increases the serum half-life is used. For example, a salvage receptor binding epitope can be incorporated into a molecule containing disclosed antigen-binding site as described, e.g., in U.S. Pat. No. 5,739,277. As used herein, the term “salvage receptor binding epitope” refers to an epitope of the Fe region of an IgG molecule (e.g., IgG1, IgG2, IgG3, or IgG4) that is responsible for increasing the in vivo serum half-life of the IgG molecule.

### Methods of Making Antigen-Binding Sites and Molecules Containing Antigen-Binding Sites

The proteins herein above can be made using recombinant DNA technology well known to a skilled person in the art.

For example, one or more nucleic acid sequences encoding a protein containing the disclosed antigen-binding site can be cloned into one or more expression vectors; the expression vectors can be stably transfected into host cells capable of expressing the gene(s). After transfection, single clones can be isolated for cell bank generation using methods known in the art, such as limited dilution, ELISA, FACS, microscopy, or Clonepix. Clones can be cultured under conditions suitable for bio-reactor scale-up and maintained expression of a protein comprising an antigen-binding site disclosed herein. The protein can be isolated and purified using methods known in the art, such as centrifugation, depth filtration, cell lysis, homogenization, freeze-thawing, affinity purification, gel filtration, ion exchange chromatography, hydrophobic interaction exchange chromatography, and mixed-mode chromatography.

Accordingly, also provided herein are isolated nucleic acids encoding antigen-binding sites and molecules (e.g., proteins) containing antigen-binding sites, vectors and host cells comprising the nucleic acid, and recombinant techniques for the production.

In certain embodiments, provided are one or more isolated nucleic acids comprising sequences encoding an immunoglobulin heavy chain and/or immunoglobulin light chain variable region of any antibody disclosed herein, and one or more expression vectors that express the immunoglobulin heavy chain and/or immunoglobulin light chain variable region of any antibody disclosed herein. Also provided are host cells comprising one or more of the foregoing expression vectors and/or isolated nucleic acids.

In some embodiments, antigen-binding sites and molecules (e.g., proteins) containing antigen-binding sites of the present disclosure may be fused to another agent, e.g., another therapeutic agent. Construction of fusion proteins is within ordinary skill in the art.

### Monoclonal Antibodies

Monoclonal antibodies may be made using the hybridoma method first described by, or may be made by recombinant DNA methods (U.S. Pat. No. 4,816,567).

In the hybridoma method, a mouse or other appropriate host animal, such as a hamster, is immunized to elicit lymphocytes that produce or are capable of producing antibodies that will specifically bind to the protein used for immunization. Alternatively or additionally, lymphocytes may be immunized in vitro. After immunization, lymphocytes are isolated and then fused with a myeloma cell line using a suitable fusing agent, such as polyethylene glycol, to form a hybridoma cell.

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Examples of suitable fusion partner include, but are not limited to, myeloma cells are those that fuse efficiently, support stable high-level production of antibody by the selected antibody-producing cells, and are sensitive to a selective medium that selects against the unfused parental cells. Examples of suitable myeloma cell lines include, but are not limited to, are murine myeloma lines, such as those derived from MOPC-21 and MPC-11 mouse tumors available from the Salk Institute Cell Distribution Center, San Diego, Calif USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies.

According to the hybridoma method, culture medium in which hybridoma cells are growing is then assayed for production of monoclonal antibodies directed against the antigen. For example, the binding specificity of monoclonal antibodies produced by hybridoma cells may be determined by immunoprecipitation or by an in vitro binding assay, such as radioimmunoassay (RIA) or enzyme-linked immunosorbent assay (ELISA).

The binding affinity of the monoclonal antibody can, for example, be determined by a Scatchard analysis.

Once hybridoma cells that produce antibodies of the desired specificity, affinity, and/or activity are identified, clones may be subcloned, e.g., by limiting dilution procedures and grown by standard methods. Suitable culture media for this purpose include, for example, D-MEM or RPMI-1640 medium. In addition, the hybridoma cells may be grown in vivo as ascites tumors in an animal, e.g., by i.p. injection of the cells into mice.

Monoclonal antibodies secreted by the subclones are suitably separated from the culture medium, ascites fluid, or serum by conventional antibody purification procedures such as, for example, affinity chromatography (e.g., using protein A or protein G-Sepharose®) or ion-exchange chromatography, hydroxylapatite chromatography, gel electrophoresis, dialysis, etc.

DNA encoding the monoclonal antibodies can be readily isolated and sequenced using conventional procedures (e.g., by using oligonucleotide probes that are capable of binding specifically to genes encoding the heavy and light chains of murine antibodies). Hybridoma cells can serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells.

In certain embodiments, monoclonal antibodies or antibody fragments are isolated from antibody phage libraries. High affinity (nM range) human antibodies can be produced, e.g., by chain shuffling. Combinatorial infection and in vivo recombination may provide strategies for constructing very large phage libraries. These techniques are viable alternatives to traditional monoclonal antibody hybridoma techniques for isolation of monoclonal antibodies.

DNA that encodes the antibody may be modified to produce chimeric or fusion antibody polypeptides, for example, by substituting human heavy chain and light chain constant domain (CH and CL) sequences for the homologous murine sequences (see, e.g., U.S. Pat. No. 4,816,567), or by fusing the immunoglobulin coding sequence with all or part of the coding sequence for a non-immunoglobulin polypeptide (heterologous polypeptide).

### Human Antibodies and Phage Display Methodology

Human antibodies can be generated by methods known in the art, including methods described herein. For example, it is possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., U.S. Pat. Nos. 5,545,806, 5,569,825, 5,591,669; 5,545,807; and WO 97/17852.

Alternatively, phage display technology can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats. Several sources of V-gene segments can be used for phage display, e.g., from random combinatorial librarues of V genes such as libraries derived from the spleens of immunized mice. A repertoire of V genes from unimmunized human donors can be constructed and antibodies to a diverse array of antigens (including self-antigens) can be isolated essentially following methods described in the art. See, e.g., U.S. Pat. Nos. 5,565,332 and 5,573,905.

Human antibodies may also be generated by in vitro activated B cells (see, e.g., U.S. Pat. Nos. 5,567,610 and 5,229,275).

Competition assays for determining whether an antibody binds to the same epitope as, or competes for binding with a disclosed antibody are known in the art. Exemplary competition assays include immunoassays (e.g., ELISA assays, RIA assays), surface plasmon resonance (e.g., BIAcore analysis), bio-layer interferometry, and flow cytometry.

Typically, a competition assay involves the use of an antigen bound to a solid surface or expressed on a cell surface, a test antibody, and a reference antibody. The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (e.g., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated human antibodies contain one or more somatic mutations. In these cases, antibodies can be modified to a human germline sequence to optimize the antibody (e.g., by a process referred to as germlining).

Generally, an optimized antibody has at least the same, or substantially the same, affinity for the antigen as the non-optimized (or parental) antibody from which it was derived. For example, in certain embodiments, an optimized antibody has a higher affinity for the antigen when compared to the parental antibody.

### Pharmaceutical Compositions

In certain embodiments, provided molecules containing disclosed antigen-binding sites are incorporated together with one or more pharmaceutically acceptable carriers into a pharmaceutical composition suitable for administration to a subject. As used herein, “pharmaceutically acceptable carrier” refers to any of a variety of solvents, dispersion media, coatings, antibacterial and antifungal agents, isotonic and absorption delaying agents, and the like that are physiologically compatible. Examples of pharmaceutically acceptable carriers include, but are not limited to, water, saline, phosphate buffered saline, dextrose, glycerol, ethanol and the like, as well as combinations thereof.

In some embodiments, pharmaceutical compositions comprise one or more tonicity agents or stabilizers. Non-limiting examples of such tonicity agents or stabilizers include sugars (e.g., sucrose), polyalcohols (e.g., mannitol or sorbitol), and sodium chloride.

In some embodiments, pharmaceutical compositions comprise one or more bulking agents and/or lyoprotectants (e.g., mannitol or glycine), buffers (e.g., phosphate, acetate, or histidine buffers), surfactants (e.g., polysorbates), antioxidants (e.g., methionine), and/or metal ions or chelating agents (e.g., ethylenediaminetetraacetic acid (EDTA)).

In some embodiments, pharmaceutical compositions comprise one or more auxiliary substances such as wetting or emulsifying agents, preservatives (e.g., benzyl alcohol) or buffers, which may enhance the shelf life and/or effectiveness of immunoconjugates disclosed herein.

Pharmaceutical compositions may be provided in any of a variety of forms. These include, for example, liquid, semi-solid and solid dosage forms, such as liquid solutions (e.g., injectable and infusible solutions), dispersions or suspensions, tablets, pills, powders, liposomes and suppositories. Suitability of certain forms may depend on the intended mode of administration and therapeutic application.

In some embodiments, pharmaceutical compositions are in the form of injectable or infusible solutions.

Pharmaceutical compositions are typically sterile and stable under conditions of manufacture, transport, and storage. Pharmaceutical compositions may be formulated as, for example, a solution, microemulsion, dispersion, liposome, or other ordered structure. In some embodiments, a pharmaceutical composition is formulated as a structure particularly suitable for high drug concentration. For example, sterile injectable solutions can be prepared by incorporating a therapeutic agent (e.g., immunoconjugate) in a desired amount in an appropriate solvent with one or a combination of ingredients enumerated herein, optionally followed by sterilization (e.g., filter sterilization). Generally, dispersions may be prepared by incorporating an immunoconjugate into a sterile vehicle that contains a basic dispersion medium and other ingredient(s) such as those additional ingredients mentioned herein. In the case of sterile powders for the preparation of sterile injectable solutions, examples of preparation methods include vacuum drying and freeze-drying to yield a powder of the immunoconjugate and any additional desired ingredient(s), e.g., from a previously sterile-filtered solution thereof.

Proper fluidity of a solution can be maintained, for example, by the use of a coating such as lecithin, by maintaining certain particle sizes (e.g., in the case of dispersions), and/or by using surfactants. Prolonged absorption of injectable compositions can be brought about, e.g., by including in the composition an agent that delays absorption (for example, monostearate salts and/or gelatin).

### Methods of Treatment

The present application provides methods for treating a disease or disorder using a therapeutic agent (e.g., protein, such as antibody or antigen-binding fragment thereof) or pharmaceutical composition described herein. Methods of treatment disclosed herein generally comprise a step of administering a therapeutically effective amount of a disclosed therapeutic agent containing antigen-binding site or a pharmaceutical composition thereof to a subject (e.g., a human subject) in need thereof.

In some embodiments, the disease or disorder is an autoimmune disease. Examples of autoimmune diseases include, not are not limited to, Behect disease, systemic lupus erythrematosus, multiple sclerosis (systemic scleroderma and progressive systemic scleroderma), scleroderma, polymyositis, dermatomyositis, periarteritis nodosa (polyarteritis nodosa and microscopic polyangiitis), aortitis syndrome (Takayasu arteritis), malignant rheumatoid arthritis, rheumatoid arthritis, Wegner's granulomatosis, mixed connective tissue disease, Sjogren syndrome, adult-onset Still's disease, allergic granulomatous angiitis, hypersensitivity angiitis, Cogan's syndrome, RS3PE, temporal arteritis, polymyalgia rheumatica, fibromyalgia syndrome, antiphospholipid antibody syndrome, eosinophilic fasciitis, IgG4-related diseases (e.g., primary sclerosing cholangitis and autoimmune pancreatitis), Guillain-Barre syndrome, myasthenia gravis, chronic atrophic gastritis, autoimmune hepatitis, primary biliary cirrhosis, aortitis syndrome, Goodpasture's syndrome, rapidly progressive glomerulonephritis, megaloblastic anemia, autoimmune hemolytic anemia, autoimmune neutropenia, idiopathic thrombocytopenic purpura, Graves' disease (hyperthyroidism), Hashimoto's thyroiditis, autoimmune adrenal insufficiency, primary hypothyroidism, idiopathic Addison's disease (chronic adrenal insufficiency), type I diabetes mellitus, chronic discoid lupus erythematosus, localized scleroderma, psoriasis, psoriatic arthritis, pemphigus, pemphigoid, herpes gestationis, linear IgA bullous skin disease, epidermolysis bullosa acquisita, alopecia areata, vitiligo, Harada disease, autoimmune optic neuropathy, idiopathic azoospermia, recurrent fetal loss, and inflammatory bowel diseases (ulcerative colitis and Crohn's disease). The prescription of the present invention can also be applied to prophylaxis or therapy of graft-versus-host disease (GVHD).

In some embodiments, the disease or disorder is an allergy or inflammatory disease. examples of allergies and inflammatory diseases include, not are not limited to, acid reflux/heartburn, acne, acne vulgaris, allergies and sensitivities, Alzheimer's disease, asthma, atherosclerosis and vascular occlusive disease, optionally atherosclerosis, ischaemic heart disease, myocardial infarction, stroke, peripheral vascular disease, or vascular stent restenosis, autoimmune diseases, bronchitis, cancer, carditis, cataracts, celiac disease, chronic pain, chronic prostatitis, cirrhosis, colitis, connective tissue diseases, optionally systemic lupus erythematosus, systemic sclerosis, polymyositis, dermatomyositis, or Sjogren's syndrome, corneal disease, crohn's disease, crystal arthropathies, optionally gout, pseudogout, calcium pyrophosphate deposition disease, dementia, dermatitis, diabetes, dry eyes, eczema, edema, emphysema, fibromyalgia, gastroenteritis, gingivitis, glomerulonephritis, heart disease, hepatitis, high blood pressure, hypersensitivities, inflammatory bowel diseases, inflammatory conditions including consequences of trauma or ischaemia, insulin resistance, interstitial cystitis, iridocyclitis, iritis, joint pain, arthritis, rheumatoid arthritis, lyme disease, metabolic syndrome (syndrome x), multiple sclerosis, myositis, nephritis, obesity, ocular diseases including uveitis, osteopenia, osteoporosis, parkinson's disease, pelvic inflammatory disease, periodontal disease, polyarteritis, polychondritis, polymyalgia rheumatica, psoriasis, reperfusion injury, rheumatic arthritis, rheumatic diseases, optionally rheumatoid arthritis, osteoarthritis, or psoriatic arthritis, rheumatoid arthritis, sarcoidosis, scleroderma, sinusitis, sjogren's syndrome, spastic colon, spondyloarthropathies, optionally ankylosing spondylitis, reactive arthritis, or reiter's syndrome, systemic candidiasis, tendonitis, transplant rejection, vaginitis, vascular diseases including atherosclerotic vascular disease, vasculitides, optionally polyarteritis nodosa, Wegener's granulomatosis, churg-strauss syndrome, or vasculitis.

Therapeutically effective amounts may be administered via a single dose or via multiple doses (e.g., at least two, at least three, at least four, at least five, at least six, at least seven, at least eight, at least nine, or at least ten doses). When administered via multiple doses, any of a variety of suitable therapeutic regimens may be used, including administration at regular intervals (e.g., once every other day, once every three days, once every four days, once every five days, thrice weekly, twice weekly, once a week, once every two weeks, once every three weeks, etc.).

The dosage regimen (e.g., amounts of each therapeutic, relative timing of therapies, etc.) that is effective in methods of treatment may depend on the severity of the disease or condition and the weight and general state of the subject. For example, the therapeutically effective amount of a particular composition comprising a therapeutic agent applied to mammals (e.g., humans) can be determined by the ordinarily-skilled artisan with consideration of individual differences in age, weight, and the condition of the mammal Therapeutically effective and/or optimal amounts can also be determined empirically by those of skill in the art.

The molecule containing antigen-binding site or pharmaceutical composition thereof may be administered by any of a variety of suitable routes, including, but not limited to, systemic routes such as parenteral (e.g., intravenous or subcutaneous) or enteral routes.

In many embodiments, administering therapeutic agents containing antigen-binding sites or pharmaceutical compositions thereof in accordance with provided methods results in depletion of cells involved in the disease to be treated. In some embodiments, the cell expresses an antigen to which the antigen-binding sites comprised in the therapeutic agents bind. In some embodiments, the cell is an immune cell, e.g., a T cell, a B cell, a macrophage, a natural killer (NK) cell, a dendritic cell (DC), a monocyte, a neutrophil, a fibroblast, or an epithelial cell. In some embodiments, the cell is an activated cell, e.g., an antigen-activated T cell or B cell.

### List of Exemplary Embodiments

The invention is further described by the following non-limiting exemplary embodiments:
Embodiment 1. A method of treating an autoimmune disease in a subject in need thereof, the method comprising administering to the subject a therapeutic agent comprising one or more antigen-binding sites,
   wherein the antigen-binding site is capable of binding an antigen on a cell involved in the autoimmune disease selected from PD-1, PD-L1, or PD-L2,
   and wherein the administering of the therapeutic agent comprising one or more antigen-binding sites results in depleting the cell.
Embodiment 2. A method of treating an autoimmune disease in a subject in need thereof, the method comprising administering to the subject a therapeutic agent comprising one or more antigen-binding sites,
   wherein the antigen-binding site is capable of binding an antigen on a cell involved in the autoimmune disease selected from PD-1, PD-L1, PD-L2, CTLA-4, a C-type lectin (CLEC), a Siglec, a tumor necrosis factor (TNF) receptor superfamily member, 4-1B ligand (4-1BBL), OX40 ligand (OX40L), CD40 ligand (CD40L), CD30 ligand (CD30L), CD70 ligand (CD70L), CD27 ligand (CD27L), TIM-3, LAG-3, BTLA, KLRG1, 234, CD244, CD19, CD20, CD22, CD28, CD38, CD39, CD72, CD73, CD79A, CD79B, glycoprotein 130 (gp130), BCMA, BAFF receptor (BAFF-R), TACI, integrin alpha 4, integrin beta 7, FCGR2B, ICOS ligand, CD138, SLAMF7, a L1LR family member, fibroblast activated protein alpha (FAP), DLK-1, CD26, TE-7, CD29, PDGRF alpha, TGF beta receptor, MAS516, CD13, or a combination thereof,
   and wherein the administering of the therapeutic agent comprising one or more antigen-binding sites results in depleting the cell.
Embodiment 3. A method of treating an allergy or inflammatory disease in a subject in need thereof, the method comprising administering to the subject a therapeutic agent comprising one or more antigen-binding sites,
   wherein the antigen-binding site is capable of binding an antigen on a cell involved in the allergy or inflammatory disease selected from PD-1, PD-L1, or PD-L2,
   and wherein the administering of the therapeutic agent comprising one or more antigen-binding sites results in depleting the cell.
Embodiment 4. A method of treating an allergy or an inflammatory disease in a subject in need thereof, the method comprises administering to the subject a therapeutic agent comprising one or more antigen-binding sites,
   wherein the antigen-binding site is capable of binding an antigen on a cell involved in the allergy or inflammatory disease selected from PD-1, PD-L1, PD-L2, CTLA-4, a C-type lectin (CLEC), a Siglec, a tumor necrosis factor (TNF) receptor superfamily member, 4-1BB ligand (4-1BBL), OX40 ligand (OX40L), CD40 ligand (CD40L), CD30 ligand (CD30L), CD70 ligand (CD7L), CD27 ligand (CD27L), TIM-3, LAG-3, BTLA, KLRG1, 2B4, CD244, CD19, CD20, CD22, CD28, CD38, CD39, CD72, CD73, CD79A, CD79B, glycoprotein 130 (gp130), BCMA, BAFF receptor, TACI, integrin alpha 4, integrin beta 7, FCGR2B, ICOS ligand, CD138, SLAMF7, and a LILR family member, fibroblast activated protein alpha (FAP), DLK-1, CD26, TE-7, CD29, PDGRF alpha, TGF beta receptor, MAS516, CD13, or a combination thereof,
   and wherein the administering of the therapeutic agent comprising one or more antigen-binding sites results in depleting the cell.
Embodiment 5. The method of any one of embodiments 1-4, wherein the cell expresses a marker selected from PD-1, PD-L1, PD-L2, CTLA-4, a CLEC, a Siglec, a TNF receptor superfamily member, 4-1BBL, OX40L, CD40L, CD30L, CD70L, CD27L, TIM-3. LAG-3, BTLA, KLRG1, 2B4, CD244, CD19, CD20, CD22, CD2S, CD38, CD39, CD72, CD⁷³, CD79A. CD79B, gp130, BCMA, BAFF receptor, TACI integrin alpha 4, integrin beta 7, FCGR2B, ICOS ligand, CD138, SLAMF7, a LILR family member, FAP, DLK-1, CD26, TE-7, CD29, PDGRF alpha, TGF beta receptor, MAS516, CD13, or a combination thereof.
Embodiment 6. The method of any one of embodiments 1-5, wherein the cell is an immune cell.
Embodiment 7. The method of any one of embodiments 1-5, wherein the cell is a T cell, a B cell, a macrophage, a natural killer (NK) cell, a dendritic cell (DC), a monocyte, a neutrophil, a fibroblast, or an epithelial cell.
Embodiment 8. The method of embodiment 6 or 7, wherein the cell is activated.
Embodiment 9. The method ofany one of embodiments 6-8, wherein the cell is an antigen-activated T cell or B cell.
Embodiment 10. The method of any one of embodiments 1-9, wherein the antigen-binding site is present as a single-chain fragment variable (scFv).
Embodiment 11. The method of any one of embodiments 1-10, wherein the antigen-binding site is present as an antibody mimetic (e.g., an Affibody, an Affilin, an Affimer, an afftin, an Alphabody, an Anticalin, an avimer, a Centyrin, a DARPin, a Fynomer, a monobody, nanobody, or a nanoCLAMP).
Embodiment 12. The method of any one of embodiments 1-11, wherein the therapeutic agent is an antibody or antigen-binding fragment thereof comprising the one or more antigen-binding sites.
Embodiment 13. The method of embodiment 12, wherein the antibody or antigen-binding fragment thereof is a Fab, a F(ab′)₂, a Fab′, an scFv, an Fv fragment, a Fd fragment, or a diabody.
Embodiment 14. The method of embodiment 12, wherein the antibody or antigen-binding fragment thereof is a humanized antibody, human antibody, multispecific antibody, or bispecific antibody, or a fragment thereof
Embodiment 15. The method of embodiments 1-11, wherein the therapeutic agent is a bispecific antibody.
Embodiment 16. The method of embodiment 15, wherein the bispecific antibody comprises a first antigen-binding site and a second antigen-binding site, wherein
   the first antigen-binding site is capable of binding PD-1, PD-L1, PD-L2, CTLA-4, BTLA, or KLRG1, and
   the second antigen-binding site is capable of binding a marker for activated T cell (e.g., TIM-3 or LAG-3).
Embodiment 17. The method of embodiment 15, wherein the bispecific antibody comprises a first antigen-binding site and a second antigen-binding site, wherein
   the first antigen-binding site is capable of binding PD-1, PD-L1, PD-L2, BCMA, or CD3S, and
   the second antigen-binding site is capable of binding a marker for activated B cell (e.g., CD19, CD20, BAFF-R, or TACI).
Embodiment 18. The method of embodiment 15, wherein the bispecific antibody comprises a first antigen-binding site and a second antigen-binding site, wherein
   the first antigen-binding site is capable of binding FAP or PDGRF alpha, and
   the second antigen-binding site is capable of binding a marker for activated fibroblast
(e.g., FAP, DLK-1, CD26, TE-7, CD29, PDGRF alpha, TGF beta receptor, MAS516, or CD13).
Embodiment 19. The method of any one of embodiments 1-18, wherein the therapeutic agent is an antibody or antigen-binding fragment thereof that comprises an antibody heavy chain constant region.
Embodiment 20. The method of embodiment 20, wherein the antibody heavy chain constant region is a human IgG heavy chain constant region.
Embodiment 21. The method of any one of embodiments 1-20, wherein the therapeutic agent is an antibody or antigen-binding fragment thereof that is conjugated or fused to a label or a second therapeutic agent.
Embodiment 22. The method of embodiment 21, wherein the second therapeutic agent is a cytotoxic small molecule or an immunotoxin.
Embodiment 23. The method of any one of embodiments 1-22, wherein the therapeutic agent is capable of inducing complement dependent cytotoxicity (CDC).
Embodiment 24. The method of any one of embodiments 1-23, wherein therapeutic agent is capable of inducing antibody-dependent cell-mediated cytotoxicity (ADCC).
Embodiment 25. The method of any one of embodiments 1-24, wherein therapeutic agent is capable of inducing antibody-dependent cellular phagocytosis (ADCP).
Embodiment 26. The method of any one of embodiments 1-25, wherein the therapeutic agent is afucosylated.
Embodiment 27. The method of embodiment 25, wherein the therapeutic agent is at least about 75% afucosylated.
Embodiment 28. The method of embodiment 26 or 27, wherein the therapeutic agent is capable of depleting an immune cell.
Embodiment 29. The method of any one of embodiments 1-28, wherein therapeutic agent is capable of agonizing PD-1.
Embodiment 30. The method of any one of embodiments 1-29, wherein the therapeutic agent comprises an amino acid sequence selected from any one of SEQ ID NOs: 1-91, or a fragment thereof.
Embodiment 31. The method of any one of embodiments 1-29, wherein the therapeutic agent comprises at least one amino acid sequence selected from any one of SEQ ID NOs: 1-91, or a fragment thereof.
Embodiment 32. The method of any one of embodiments 1-29, wherein the therapeutic agent comprises at least two amino acid sequences selected from any one of SEQ ID NOs: 1-91, or a fragment thereof.
Embodiment 33. The method of any one of embodiments 1-29, wherein the therapeutic agent comprises at least one CDR found in a sequence selected from any one of SEQ ID NOs: 1, 5, 9, 13, 17, 21, 25, 29, 33, 37, 41, 44, 46, 48, 50 54, 58, 61, 63, 65, 67, 69, 73, 77, 80, 82, 84, 88, 92, 95, and 97.
Embodiment 34. The method of any one of embodiments 1-29, wherein the therapeutic agent comprises at least two CDRs from sequences selected from any one of SEQ ID NOs: 1, 5, 9, 13, 17, 21, 25, 29, 33, 37, 41, 44, 46, 48, 50 54, 58, 61, 63, 65, 67, 69, 73, 77, 80, 82, 84, 88, 92, 95, and 97.
Embodiment 35. The method of any one of embodiments 1-29, wherein the therapeutic agent comprises at least three CDRs from sequences selected from any one of SEQ ID NOs: 1, 5, 9, 13, 17, 21, 25, 29, 33, 37, 41, 44, 46, 48, 50 54, 58, 61, 63, 65, 67, 69, 73, 77, 80, 82, 84, 88, 92, 95, and 97.
Embodiment 36. The method of any one of embodiments 1-29, wherein the therapeutic agent comprises
   (a) the complementarity-determining region 1 (CDR1), CDR2, and/or CDR3 of a heavy chain variable region (VH) set forth in any one of SEQ ID NO: 1, 9, 17, 25, 33, 50, 69, and 84; and/or
   (b) the CDR1, CDR2, and/or CDR3 of a light chain variable region (VL) set forth in any one of SEQ ID NO: 5, 13, 21, 29, 37, 55, 73, and 88.
Embodiment 37. The method of any one of embodiments 1-29, wherein the therapeutic agent comprises
(i) (a) a VH comprising complementarity-determining regions:
   CDR-H1 comprising the amino acid sequence: SGGSIRSTRWWS (SEQ ID NO: 2);
   CDR-H2 comprising the amino acid sequence: EIYHSGSTNYNPSLKS (SEQ ID NO: 3); and
   CDR-H3 comprising the amino acid sequence: QDYGDSGDWYFDL (SEQ ID NO: 4); and/or
   (b) a VL comprising complementarity-determining regions:
   CDR-L1 comprising the amino acid sequence: TRSSGSIASNSVQ (SEQ ID NO: 6);
   CDR-L2 comprising the amino acid sequence: EDNQRPS (SEQ ID NO: 7); and
   CDR-L3 comprising the amino acid sequence: OSSDSSAVV (SEQ ID NO: 8); or
(ii) (a) a VH comprising complementarity-determining regions:
   CDR-H1 comprising the amino acid sequence: SYGIS (SEQ ID NO: 10);
   CDR-H2 comprising the amino acid sequence: WISAYNGNTNYAQKLQ (SEQ D NO: 11); and
   CDR-H3 comprising the amino acid sequence: DADYSSGSGY (SEQ ID NO: 12); and/or
   (b) a VL comprising complementarity-determining regions:
   CDR-L1 comprising the amino acid sequence: SGDATPKQYAY (SEQ ID NO: 14);
   CDR-L2 comprising the amino acid sequence: KDTERPS (SEQ ID NO: 15); and
   CDR-L3 comprising the amino acid sequence: QSADNSITYRV (SEQ ID NO: 16); or
(iii) (a) a VH comprising complementarity-determining regions:
   CDR-H1 comprising the amino acid sequence: SYY IH (SEQ ID NO: 18);
   CDR-H2 comprising the amino acid sequence: IINPRGATISYAQKFQG (SEQ ID NO: 19); and
   CDR-H3 comprising the amino acid sequence: AGIYGFDFDY (SEQ ID NO: 20); and/or
   (b) a VL comprising complementarity-determining regions:
   CDR-L1 comprising the amino acid sequence: TGTSNDVGGYNYVS (SEQ ID NO: 22);
   CDR-L2 comprising the amino acid sequence: DVTNRPS (SEQ ID NO: 23); and
   CDR-L3 comprising the amino acid sequence: SSYTIVTNFEVL (SEQ ID NO: 24); or
(iv) (a) a VH comprising complementarity-determining regions:
   CDR-H1 comprising the amino acid sequence: SGAYYWS (SEQ ID NO: 26);
   CDR-H2 comprising the amino acid sequence: YIYYNGNTYYNPSLRS (SEQ ID NO: 27); and
   CDR-H3 comprising the amino acid sequence: ASDYVWGGYRYMDAFDI (SEQ ID NO: 28); and/or
   (b) a VL comprising complementarity-determining regions:
   CDR-L1 comprising the amino acid sequence: SGSNSNIGSNSVN (SEQ ID NO: 30);
   CDR-L2 comprising the amino acid sequence: GNNQRPS (SEQ ID NO: 31); and
   CDR-L3 comprising the amino acid sequence: AAWDDSLNGP (SEQ ID NO: 32); or
(v) (a) a VH comprising complementarity-determining regions;
   CDR-H1 comprising the amino acid sequence: DYYIN (SEQ ID NO: 34);
   CDR-H2 comprising the amino acid sequence: W IYPGSVN TKYNEKFRG (SEQ ID NO: 35); and
   CDR-H3 comprising the amino acid sequence: YSNWFFDV (SEQ ID NO: 36); and/or
   (b) a VL comprising complementarity-determining regions:
   CDR-L1 comprising the amino acid sequence: RASQEISGYLS (SEQ ID NO: 38);
   CDR-L2 comprising the amino acid sequence: ASTLDS (SEQ ID NO: 39); and
   CDR-L3 comprising the amino acid sequence: LQYASYPYT (SEQ ID NO: 40); or
(vi) (a) a VH comprising complementarity-determining regions:
   CDR-H1 comprising the amino acid sequence: DHAIH (SEQ ID NO: 51);
   CDR-H2 comprising the amino acid sequence: YISPGYGDIEYNEKFKG (SEQ ID NO: 52); and
   CDR-H3 comprising the amino acid sequence: TTGY (SEQ ID NO: 53); and/or
   (b) a VL comprising complementarity-determining regions:
   CDR-L1 comprising the amino acid sequence: KSSQSLLNSDGKTYLN (SEQ ID NO: 55),
   CDR-L2 comprising the amino acid sequence: LVSELDS (SEQ ID NO: 56); and
   CDR-L3 comprising the amino acid sequence: WQGTHFPBH (SEQ ID NO: 57), or
(vii) (a) a VH comprising complementarity-determining regions:
   CDR-H1 comprising the amino acid sequence: SFGMS (SEQ ID NO: 70);
   CDR-H2 comprising the amino acid sequence: TISGGGSNTYYPDSVKG (SEQ ID NO: 71); and
   CDR-H3 comprising the amino acid sequence: IYDVAWF (SEQ ID NO: 72); and/or
   (b) a VL comprising complementarity-determining regions:
   CDR-L1 comprising the amino acid sequence: RASESVDNSGISFMN (SEQ ID NO: 74);
   CDR-L2 comprising the amino acid sequence: AASNQGS (SEQ ID NO: 75); and
   CDR-L3 comprising the amino acid sequence: QQSKEVPWT (SEQ ID NO: 76); or
(viii) (a) a VH comprising complementarity-determining regions:
   CDR-H1 comprising the amino acid sequence: SYAIS (SEQ ID NO: 85);
   CDR-H2 comprising the amino acid sequence: TITGGGRNTYYPDSVtKG (SEQ ID NO: 86); and
   CDR-H3 comprising the amino acid sequence: QGYDGYTWFAY (SEQ ID NO: 87); and/or
   (b) a VL comprising complementarity-determining regions:
   CDR-L1 comprising the amino acid sequence: PASESVDNSGISFMN (SEQ ID NO: 89),
   CDR-L2 comprising the amino acid sequence: AASNPGS (SEQ ID NO: 90); and
   CDR-L3 comprising the amino acid sequence: QQSKEVPWT (SEQ ID NO: 91).
Embodiment 38. The method of any one of embodiments 1-29 and 36-37, wherein the therapeutic agent comprises
(i) a VH comprising an amnio acid sequence having at least 85% identity to SEQ ID NO: 1; and/or a VL comprising an amnio acid sequence having at least 85% identity to SEQ ID NO: 5; or
(ii) a VH comprising an amnio acid sequence having at least 85% identity to SEQ ID NO: 9; and/or a VL comprising an amnio acid sequence having at least 85% identity to SEQ ID NO: 13; or
(iii) a VH comprising an amnio acid sequence having at least 85% identity to SEQ ID NO: 17; and/or a VL comprising an amnio acid sequence having at least 85% identity to SEQ ID NO: 21; or
(iv) a VH comprising an amnio acid sequence having at least 85% identity to SEQ ID NO: 25; and/or a VL comprising an amnio acid sequence having at least 85% identity to SEQ ID NO: 29; or
(v) a VH comprising an amnio acid sequence having at least 85% identity to SEQ ID NO: 33; and/or a VL comprising an amnio acid sequence having at least 85% identity to SEQ ID NO: 37; or
(vi) a VH comprising an amnio acid sequence having at least 85% identity to the VH of a HC set forth in any one of SEQ ID NO: 41 or 44; and/or a VL comprising an amnio acid sequence having at least 85% identity to the VL of a LC set forth in any one of SEQ ID NO: 46 or 48; or
(vii) a VH comprising an amnio acid sequence having at least 85% identity to SEQ ID NO: 50; and/or a VL comprising an amnio acid sequence having at least 85% identity to SEQ ID NO: 55; or
(viii) a VII comprising an amnio acid sequence having at least 85% identity to the VH of a HC set forth in any one of SEQ ID NOs: 58, 61, 63, and 65; and/or a VL comprising an amnio acid sequence having at least 85% identity to the VL of a LC set forth SEQ ID NO: 67; or
(ix) a VH comprising an amnio acid sequence having at least 85% identity to SEQ ID NO: 69; and/or a VL comprising an amnio acid sequence having at least 85% identity to SEQ ID NO: 73; or
(x) a VII comprising an amnio acid sequence having at least 85% identity to the VH of a HC set forth in SEQ ID NO: 77; and/or a VL comprising an amnio acid sequence having at least 85% identity to the VL of a LC set forth in any one of SEQ ID NO: 80 or 82; or
(xi) a VH comprising an amnio acid sequence having at least 85% identity to SEQ ID NO: 84; and/or a VL comprising an amnio acid sequence having at least 85% identity to SEQ ID NO: 88; or
(xii) a VH comprising an amnio acid sequence having at least 85% identity to the VH of a HC set forth in SEQ ID NO: 92; and/or a VL comprising an amnio acid sequence having at least 85% identity to the VL of a LC set forth in any one of SEQ ID NO: 95 or 97
Embodiment 39. The method of any one of embodiments 1-29 and 36-37, wherein the therapeutic agent comprises
(i) a HC comprising CDR-H1, CDR-H2, and CDR-H3 of a HC set forth in only one of SEQ ID NO.: 41 or 44, and having at least 85% amino acid sequence identity thereof; and/or
   a LC comprising CDR-L1, CDR-L2, and CDR-L3 of a LC set forth in any one of SEQ ID NO: 46 or 48, and having at least 85% amino acid sequence thereof; or
(ii) a HC comprising CDR-H1, CDR-H2, and CDR-H3 of a HC set forth in any one of SEQ ID NOs: 58, 61, 63, and 65, and having at least 85% amino acid sequence identity thereof; and/or
   a LC comprising CDR-L1, CDR-L2, and CDR-L3 of a LC set forth in SEQ ID NO: 67, and having at least 85% amino acid sequence identity to SEQ ID NO: 67; or
(iii) a HC comprising CDR-1, CDR-H2, and CDR-H3 of a HC set forth in SEQ ID NO: 77, and having at least 85% amino acid sequence identity to SEQ ID NO: 77; and/or
   a LC comprising CDR-L1, CDR-L2, and CDR-L3 of a LC set forth in any one of SEQ ID NO: 80 or 82, and having at least 85% amino acid sequence thereof; or
(iv) a HC comprising CDR-H1, CDR-H2, and CDR-H3 of a HC set forth in SEQ ID NO: 92, and having at least 85% amino acid sequence identity to SEQ ID NO: 92; and/or
   a LC comprising CDR-L1, CDR-L2, and CDR-L3 of a LC set forth in any one of SEQ ID NO: 95 or 97, and having at least 85% amino acid sequence thereof.
Embodiment 40. The antigen-binding site of embodiment 38 or 39, wherein the sequence identity is at least 90%, 95%, 96%, 97%, 98%, or 99%.
Embodiment 41. The antigen-binding site of any one of embodiments 1-29 and 36-40, wherein the antigen-binding site comprises
(i) a VH comprising an amnio acid sequence of SEQ ID NO: 1; and/or
   a VL comprising an amnio acid sequence of SEQ ID NO: 5; or
(ii) a VH comprising an amnio acid sequence of SEQ ID NO: 9; and/or
   a VL comprising an amnio acid sequence of SEQ ID NO: 13; or
(iii) a VH comprising an amnio acid sequence of SEQ ID NO: 17; and/or
   a VL comprising an amio acid sequence of SEQ ID NO: 21; or
(iv) a VH comprising an amnio acid sequence of SEQ ID NO: 25; and/or
   a VL comprising an amnio acid sequence of SEQ ID NO: 29, or
(v) a VH comprising an amnio acid sequence of SEQ ID NO: 33; and/or
   a VL comprising an amio acid sequence of SEQ ID NO: 37; or
(vi) the VH of a HC set forth in any one of SEQ ID NO: 41 or 44; and/or
   the VL of a LC set forth in any one of SEQ ID NO: 46 or 48; or
(vii) a VH comprising an amnio acid sequence of SEQ ID NO: 50; and/or
   a VL comprising an amnio acid sequence of SEQ ID NO: 55, or
(viii) the VH of a HIC set forth in any one of SEQ ID NOs: 58, 61, 63, and 65; and/or
   the VL of a LC set forth SEQ ID NO: 67; or
(ix) a VHT comprising an amnio acid sequence of SEQ ID NO: 69; and/or
   a VL comprising an amnio acid sequence of SEQ ID NO: 73; or
(x) the VH of a H C set forth in SEQ ID NO: 77, and/or
   the VL of a LC set forth in any one of SEQ ID NO: 80 or 82; or
(xi) a VH comprising an amnio acid sequence of SEQ ID NO: 84; and/or
   a VL comprising an amnio acid sequence of SEQ ID NO: 88; or
(xii) the VH of a HC set forth in SEQ ID NO: 92; and/or
   the VL of a LC set forth in any one of SEQ ID NO: 95 or 97.
Embodiment 42. The method of embodiment 12, wherein the antibody or antigen-binding fragment thereof is afucosylated.
Embodiment 43. The method of embodiment 42, wherein the antibody or antigen binding fragment thereof is at least about 75% afucosylated.

### EXAMPLES

### Example I. Antibody Production

Human antibodies targeting an antigen on a cell involved in an autoimmune, allergic, or inflammatory disease are produced by methods known in the art.

In some embodiments, transgenic mice that are incapable of expressing functional endogenous immunoglobulins but express human immunoglobulin genes are used. In particular, the human heavy and light chain immunoglobulin gene complexes are introduced randomly or by homologous recombination into mouse embryonic stem cells. Alternatively, the human variable region, constant region, and diversity region are introduced into mouse embryonic stein cells in addition to the human heavy and light chain genes. The mouse heavy and light chain immunoglobulin genes are rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g. PD-1, PD-L1, or PD-L2). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation.

In some other embodiments, antibodies are produced by screening yeast display libraries.

### Example 2. Binding of Antibodies Toward PD-1

Target binding of PD-1 antibodies to PD-1 expressed on activated T cells was assessed by flow cytometry.

T cells were activated with anti-CD3, anti-CD28, and anti-W6/32 treatment. Activation resulted in increased expression of PD-1 on the surface of T cells. After 3 days of activation, the cells were resuspended in FACS Blocking Buffer (PBS+10% normal human serum) for 30 minutes. Following washing, cells were incubated with PD-1 test antibodies in FACS Buffer (PBS+0.5% bovine serum albumin+0.09% sodium azide) and incubated at 4° C. for 30 minutes. Cells were subsequently washed and incubated with PE-labeled anti-human secondary antibody at 4° (C for 30 minutes in the dark. Following washing, cytofluorimetric analysis was conducted using a BD FACSSymphony A3 instrument (BD Biosciences). Cells were sorted by scatter properties and separated for single cell analysis using side scatter width vs side scatter area. Antibody binding to cells was quantitated by measuring fluorescent emissions of excitation wavelength of 561 nm using a 586/15 nm filter. Data analysis was performed using FlowJo Software. All tested c-Kit antiboties were capable of binding to T cells expressing PD-1 (see Table 2),

TABLE 2
Binding of PD-1 antibodies to PD-1 expressed on T cells

| | Binds PD-1 on |
|---|---|
| Antibody | activated T cells |
| 3H4 | + |
| 2.3A9 | + |
| 20B3.1 | + |
| 1B8 | + |
| 1-17 | + |
| 1-28 | + |
| 1-33 | + |
| 1-35 | + |
| 1.A10 | + |
| 1G7 | + |
| 7G3 | + |

### Example 3. Cross-Reactivity Non-Specific Binding of PD-1 Antibodies Analyzed by ELISA

An ELISA-based assay was employed to assess the cross-reactivity and non-specific binding of PD-1 antibodies toward human PD-1, cynomongus PD-1, and PD-1 family members.

His-tagged extracellular domains of human PD-1, cynomolgus PD-1, and PD-1 family members ICOS, CD28, and CTLA-4 extracellular domains were immobilized on 96 well ELISA plates. Plates were washed with PBST (phosphate buffered saline+0.05% Tween 20) and blocked for 1 hour with 1% BSA in PBS. Following washing with PBST, test PD-1 antibodies were added to the coated plate and incubated for 2 hours at room temperature. Plates were washed and incubated with HRP-conjugated goat anti-human IgG secondary antibody for 1 hour at room temperature. Following washing, antibody binding was detected colorimetrically by adding 3,3′,5,5′-tetramethylbenzidine (TMB) and incubated at room temperature for 10-20 minutes before adding 2.5N sulfuric acid stopping solution. Absorbance at 450 nm was measured on a plate reader (PerkinElmer). Data was analyzed using Graph-Pad Prism.

Exemplary results are depicted in Table 3. All tested PD-1 antibodies were capable of binding to both human and cynomolgus PD-1 but did not cross react with PD-1 family members ICOS, CD28, or CTLA-4.

TABLE 3
Cross-reactivity of PD-1 antibodies

| | Cross Reactivity | | |
|---|---|---|---|
| Antibody | Human PD-1 | Cynomolgus PD-1 | PD-1 Family Members |
| 3H4 | + | + | − |
| 2.3A9 | + | + | − |
| 20B3.1 | + | + | − |
| 1B8 | + | + | − |
| 1-17 | + | + | − |
| 1-28 | + | + | − |
| 1-33 | + | + | − |
| 1-35 | + | + | − |
| 1.8A10 | + | + | − |
| 1G7 | + | + | − |
| 7G3 | + | + | − |

### Example 4. PD-1/PD-1F Blockade by PD-1 Antibodies Analyzed by ELSA

The ability of disclosed PD-1 antibodies to block the PD-1/PD-L1 interaction was assessed using an ELISA based assay.

Briefly, human PD-L1 was immobilized on 96 well ELISA plates. Plates were washed and blocked for 1.5 hours at 37° C. with block buffer. Plates were washed and test PD-1 antibodies, isotype controls, or a standard curve of PD-1 neutralizing antibody were added to the coated plate and incubated for 1 hour at 37° C. Plates were washed and incubated with PD-1 biotin for 1 hour at 37° C. Following washing, streptavidin-HRP was added to the plate and the plate was incubated at 37° C. for 1 hour. After a final wash, antibody binding was detected colorimetrically by adding a subrate solution and incubating for 20 minutes protected from light before adding stop solution. Absorbance at 450 nm was measured on a plate reader (PerkinElmer). Data was analyzed using GraphPad Prism.

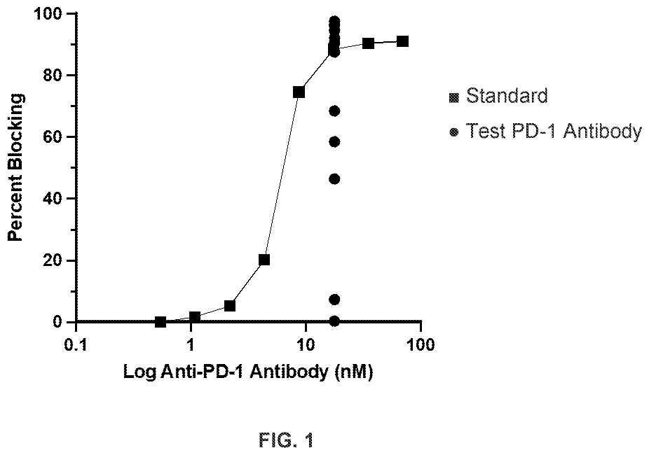

Percent blocking of tested PD-1 antibodies was determined by interpolating values obtained from each sample against the standard control curve (see FIG. 1) and summarized in Table 4. Tested antibodies showed a spectrum of activities in blocking PD-1/PD-L1 interaction. For example, PD-1 antibodies such as 1G7 and 7G3 almost completely blocked PD-1/PD-L1 interaction, while PD-1 antibodies 1-17 and 2.3A9 were non-blocking.

TABLE 4
Blockade of PD-1/PD-L1 interaction by PD-1 antibodies

| Antibody | Percent Blocking |
|---|---|
| 1G7 | 97 |
| 7G3 | 96 |
| 20B3.1 | 94 |
| 1.8A10 | 92 |
| 1B8 | 90 |
| 1-28 | 87 |
| 1-33 | 68 |
| 1-35 | 58 |
| 3H4 | 46 |
| 1-17 | 7 |
| 2.3A9 | 0 |

### Example 5. T Cell Suppression in a Mixed Lymphocyte Response by PD-1 Antibodies

The ability of disclosed PD-1 antibodies to agonize PD-1 and suppress T cells in a mixed lymphocyte response (MLR) was assessed.

Peripheral blood mononuclear cells (PBMCs) from two different donors were co-cultured in a 96 well plate. Co-culturing of PBMCs from different donors causes an allogeneic stimulus, as the allogeneic T cells from one donor produces IFN-γ after recognizing foreign MHC from the other donor.

PD-1 antibodies or isotype controls were added to the PBMC co-culture. The PBMCs alone (without antibodies) were used as controls. Plates were incubated for 72 hours and the level of IFN-γ present in the supernatant was measured by an ELISA assay.

Briefly, IFN-γ capturing antibody was immobilized on 96 well ELISA plates. Plates were washed with PBST and blocked for 1 hour with 1% BSA in PBS. Following washing with PBST, supernatants and an IFN-γ standard curve were added to the coated plate and incubated for 2 hours at room temperature. Plates were washed and incubated with HRP-conjugated detection antibody for 2 hours at room temperature. Following washing, antibody binding was detected colorimetrically by adding TMB and incubated at room temperature for 10-20 minutes before adding 2.5N sulfuric acid stopping solution. Absorbance at 450 nm was measured on a plate reader (PerkinElmer). Data was analyzed using GraphPad Prism.

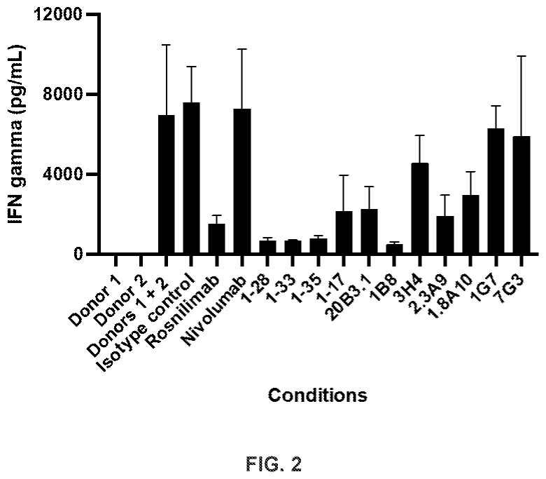

Exemplary results are shown in FIG. 2. PD-1 antibodies 1-28, 1-33, 1-35, 1-17, 20B3.1, 1B8, 3H4, 2.3A9, 1.8A10, 1G7, and 7G3 suppressed IFN-γ secretion, with some showing more enhanced suppression of IFN-γ secretion compared to PD-1 agonistic antibody rosnilimab.

### Example 6. Inhibition of T Cell Activation by Effector Function Modified PD-1 Antibodies

The ability of effector function modified PD-1 antibodies in inhibiting T cell activation was assessed A commercially available assay was used, comprising an antigen-presenting cell line (APC) and a human Jurkat reporter T cell line expressing a specific T cell receptor (TCR) and the Lucia luciferase reporter gene. In this assay, activation of the Jurkat cells leads to NFAT-mediated luminescence.

Raji-APC cells modified to express the FcγRII receptor were incubated with PD-1 antibodies with normal human IgG1 Fc or effector enhanced IgG1 Fe, isotype controls, or media for 30 minutes at 37° C., 5% CO₂. The effector enhanced IgG1 Fc contains mutations S239D/1-268) (“X3” mutations) or E233D)/G237D/H268D/P271G/A330R (“V12” mutations) that increase binding to FcγRII Following incubation, Jurkat-Lucia TCR-hPD-1 cells were added to the culture, and the plate was incubated for an additional 5.5 hours at 37° C., 5% CO₂. The cells were centrifuged at 400 g for 5 minutes and the supernatant was collected for further analysis.

The supernatant was transferred into a 96-well white plate. 50 L of QUANTI-Luc 4 Reagent was added to the plate and luminescence was read immediately using a plate reader (PerkinElmer). Data were analyzed using GraphPad Prism and relative percent inhibition was calculated compared to the signal obtained from controls (isotype control or without antibody treatment).

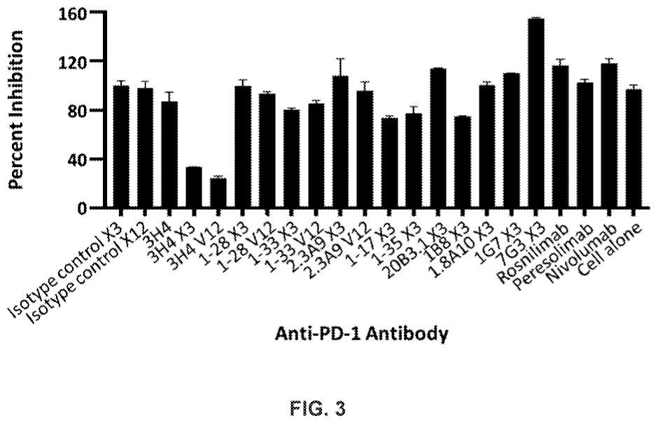

Exemplary results are shown in FIG. 3. X3 or V12 mutations enhanced the ability to inhibit Jurkat cell activation of some P1)-1 antibodies (such as 3-14) compared to PD-1 antibodies with normal IgG1 Fc and the isotype controls.

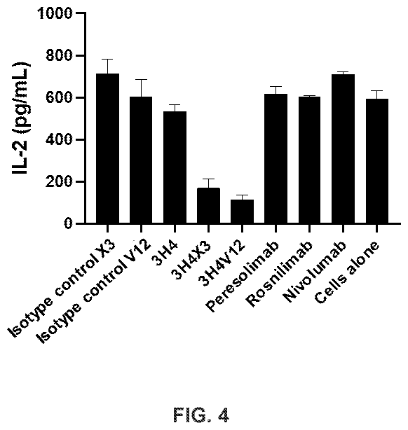

The supernatant was also analyzed for IL-2 levels by an ELISA assay, as described in Example 5, except that IL-2 capturing antibody was immobilized on 96 well ELSA plates. Exemplary results are shown in FIG. 4. 3H4 antibody containing X3 or V12 mutations significantly inhibited IL-2 secretion compared to 3H4 with normal IgG1 Fc and the controls (isotype control or without antibody treatment).

### Example 7. Inhibition of T Cell Proliferation by Effector Function Modified PD-1 Antibodies

The ability of effector function modified PD-1 antibodies in inhibiting T cell proliferation was assessed.

PBMCs were stained with CellTrace Violet (CTV) dye for 15 minutes at 37° C. Following 2 washes in cell growth media, PBMCs were plated on a 96 well plate coated with anti-CD3 and anti-CD28, added to all wells to stimulate T cells. PD-1 antibodies with normal human IgG1 Fc or effector enhanced IgG1 Fc containing X3 mutations, isotype control, or PD-L1 were added to the respective cells, and the plate was incubated for 72 hours at 37° C, 5% CO₂. Supernatants were collected for ELISA analysis. The remaining cells on the plate were washed, resuspended in FACS Blocking Buffer for 15 minutes on ice and incubated with APC labeled anti-CD4 and BV785 labeled anti-CD8 antibodies. Following washing, cytofluorimetric analysis was conducted using a BD FACS Symphony A3 instrument (BD Biosciences). CTV fluorescence intensity was measured for CD4 and CD8 T cell subsets. Data analysis was performed using FlowJo Software, and percent of proliferating cells for each T cell subtype was quantified.

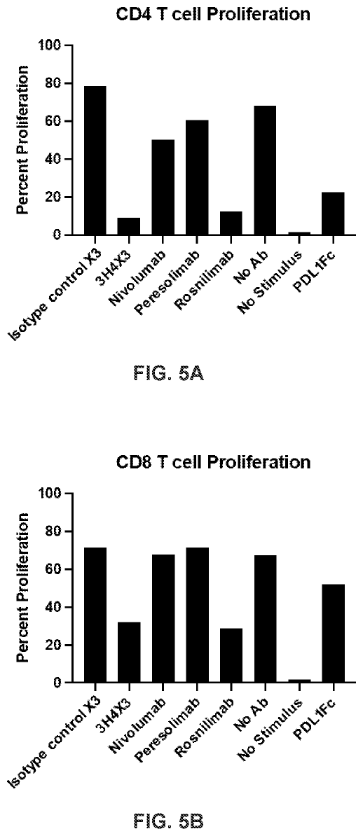

Exemplary results are shown in FIGS. 5A and 5B. Tested PD-1 antibody including X3 mutations that increase binding to FcγRII inhibited CD4 and CD8 T cell proliferation.

### Example 8. Inhibition of IFN-γ Secretion by Effector Function Modified PD-1 Antibodies

The ability of effector function modified PD-1 antibodies in inhibiting IFN-γ secretion was assessed.

Supernatants from stimulated PBMCs stained with CTV and incubated with PD-1 antibodies, isotype control, or PD-L1 were analyzed for IFN-γ secretion by an ELISA, as described in Example 5.

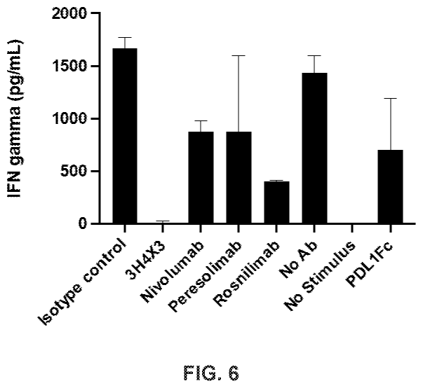

Exemplary results are shown in FIG. 6. Tested PD-1 antibody including X3 mutations that increase binding to FcγRII inhibited IFN-γ secretion more significantly than the controls and PD-1 antibodies (agonistic or antagonist) with normal IgG1 Fc.

### Sequence Annex

In some sequences, variable regions are bolded, and CDRs are underlined.

TABLE 5
Sequences of exemplary antigen-binding sites capable of binding PD-1

| | | |
|---|---|---|
| SEQ | | |
| ID NO | Description | Sequence |

1-17

| | | |
|---|---|---|
| 1 | Heavy chain variable | QVQLQESGPGVVKPSGTLSLTCAISGGSIGSGGSIRSTRWWSWVRQSP |
| | region | GKGLEWIGEIYHSGSTNYNPSLKSRVTISLDKSRNHFSLRINSVTAAD |
| | | TAVYYCARQDYGDSGDWYFDLWGKGTMVTVSS |
| 2 | HCDR1 (Kabat) | SGGSIRSTRWWS |
| 3 | HCDR2 (Kabat) | EIYHSGSTNYNPSLKS |
| 4 | HCDR3 (Kabat) | QDYGDSGDWYFDL |
| 5 | Light chain variable | NFMLTQPHSVSESPGKTVTISCTRSSGSIASNSVQWYQQRPGSSPTTV |
| | region | IYEDNQRPSGVPDRFSGSIDSSSNSASLTVSGLKTEDEADYYCQSSDS |
| | | SAVVFGSGTKLTVL |
| 6 | LCDR1 (Kabat) | TRSSGSIASNSVQ |
| 7 | LCDR2 (Kabat) | EDNQRPS |
| 8 | LCDR3 (Kabat) | QSSDSSAVV |

1-28

| | | |
|---|---|---|
| 9 | Heavy chain variable | EVQLVQSGAEVKKPGASVKVSCKASGYRFTSYGISWVRQAPGQGLEWM |
| | region | GWISAYNGNTNYAQKLQGRVTMTTDTSTNTAYMELRSLRSDDTAVYYC |
| | | ARDADYSSGSGYWGQGTLVTVSS |
| 10 | HCDR1 (Kabat) | SYGIS |
| 11 | HCDR2 (Kabat) | WISAYNGNTNYAQKLQ |
| 12 | HCDR3 (Kabat) | DADYSSGSGY |
| 13 | Light chain variable | SYELTQPPSVSVSPGQTARITCSGDALPKQYAYWYQQKPGQAPVMVIY |
| | region | KDTERPSGIPERFSGSSSGTKVTLTISGVQAEDEADYYCQSADNSITY |
| | | RVFGGGTKVTVL |
| 14 | LCDR1 (Kabat) | SGDALPKQYAY |
| 15 | LCDR2 (Kabat) | KDTERPS |
| 16 | LCDR3 (Kabat) | QSADNSITYRV |

1-33

| | | |
|---|---|---|
| 17 | Heavy chain variable | QVQLVQSGAEVKKPGASVRVSCKASGYTITSYYIHWVRQAPGQGLEWM |
| | region | GIINPRGATISYAQKFQGRVTMTRDTSTSTVYMELRNLKSEDTALYYC |
| | | ATAGIYGFDFDYWGRGTLVTVSS |
| 18 | HCDR1 (Kabat) | SYYIH |
| 19 | HCDR2 (Kabat) | IINPRGATISYAQKFQG |
| 20 | HCDR3 (Kabat) | AGIYGFDFDY |
| 21 | Light chain variable | QSALTQPASVSGSPGQSITISCTGTSNDVGGYNYVSWYQHHPGKAPKL |
| | region | IIYDVTNRPSGVSDRFSGSKSGNTASLTISGLLAEDEGDYYCSSYTIV |
| | | TNFEVLFGGGTKLTV |
| 22 | LCDR1 (Kabat) | TGTSNDVGGYNYVS |
| 23 | LCDR2 (Kabat) | DVTNRPS |
| 24 | LCDR3 (Kabat) | SSYTIVTNFEVL |

1-35

| | | |
|---|---|---|
| 25 | Heavy chain variable | QVQLQESGPGLVKPSQTLSLTCTVSGGSISSGAYYWSWIRQHPGKGLE |
| | region | WIGYIYYNGNTYYNPSLRSLVTISVDASKNQFSLKLSSVTAADTAVYY |
| | | CARASDYVWGGYRYMDAFDIWGRGTLITVSS |
| 26 | HCDR1 (Kabat) | SGAYYWS |
| 27 | HCDR2 (Kabat) | YIYYNGNTYYNPSLRS |
| 28 | HCDR3 (Kabat) | ASDYVWGGYRYMDAFDI |
| 29 | Light chain variable | QSVITQPPSASGTPGQRVTISCSGSNSNIGSNSVNWYQQLPGTAPKLL |
| | region | IYGNNQRPSGVPDRFSGSKSGTSASLAISGLQSENEADYYCAAWDDSL |
| | | NGPVFGRGTKVTVLGE |
| 30 | LCDR1 (Kabat) | SGSNSNIGSNSVN |
| 31 | LCDR2 (Kabat) | GNNQRPS |
| 32 | LCDR3 (Kabat) | AAWDDSINGP |

Murine 3H4

| | | |
|---|---|---|
| 33 | Heavy chain variable | QIQLQQSGPELVKPGASVKISCKASGYTFTDYYINWVKQKPGQGLEWI |
| | region | GWIYPGSVNTKYNEKFRGKATILTVDTSSSTAYIQLSGLTSEDTAVYFC |
| | | ARYSNWFEDVWGAGTAVTVS |
| 34 | HCDR1 (Kabat) | DYYIN |
| 35 | HCDR2 (Kabat) | WIYPGSVNTKYNEKFRG |
| 36 | HCDR3 (Kabat) | YSNWFEDV |
| 37 | Light chain variable | DIVMTQSPSSLSASLGERVSLTCRASQEISGYLSWLQQKPDGTIKRLI |
| | region | YAASTLDSGVPKRFSGSRSGSDYSLSISSLESEDFADYYCLQYASYPY |
| | | TFGGGTKLEIKR |
| 38 | LCDR1 (Kabat) | RASQEISGYLS |
| 39 | LCDR2 (Kabat) | AASTLDS |
| 40 | LCDR3 (Kabat) | LQYASYPYT |

Humainzed 3H4 heavy chain 2 (h3H4-H2)

| | | |
|---|---|---|
| 41 | h3H4-H2, IgG1 | QVQLVQSGAEVKKPGASVKVSCKASGYTFTDYYINWVRQAPGQGLEWM |
| | (Fc or constant region | GWIYPGSVNTKYAQKFQGRVTMTVDTSTSTAYMELSSLRSEDTAVYYC |
| | may be of any IgG | ARYSNWFFDVWGQGTTVTVSSASTKGPSVFPLAPSSKSTSGGTAALGC |
| | isotype | LVKDYFPEPVTVSWNSGALTSGVHTFPAVLQSSGLYSLSSVVTVPSSS |
| | and/or contain any | LGTQTYICNVNHKPSNTKVDKRVEPKSCDKTHTCPPCPAPELLGGPSV |
| | modifications or | FLFPPKPKDTLMISRTPEVTCVVVDVSHEDPEVKFNWYVDGVEVHNAK |
| | mutations) | TKPREEQYNSTYRVVSVLTVLHQDWLNGKEYKCKVSNKALPAPIEKTI |
| | | SKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPG |
| 42 | Heavy chain variable | QVQLVQSGAEVKKPGASVKVSCKASGYTFTDYYINWVRQAPGQGLEWM |
| | region | GWIYPGSVNTKYAQKFQGRVTMTVDTSTSTAYMELSSLRSEDTAVYYC |
| | | ARYSNWFEDVWGQGTTVTVSS |
| 34 | HCDR1 (Kabat) | DYYIN |
| 43 | HCDR2 (Kabat) | WIYPGSVNTKYAQKFQG |
| 36 | HCDR3 (Kabat) | YSNWFFDV |

Humainzed 3H4 heavy chain 3 (h3H4-H3)

| | | |
|---|---|---|
| 44 | h3H4-H3, IgG1 | QVQLVQSGAEVKKPGASVKISCKASGYTFTDYYINWVRQAPGQGLEWI |
| | (Fc or constant region | GWIYPGSVNTKYAQKFQGRATLTVDTSTSTAYIELSSLRSEDTAVYYC |
| | may be of any IgG | ARYSNWFFDVWGQGTTVTVSSASTKGPSVFPLAPSSKSTSGGTAALGC |
| | isotype | LVKDYFPEPVTVSWNSGALTSGVHTFPAVLQSSGLYSLSSVVTVPSSS |
| | and/or contain any | LGTQTYICNVNHKPSNTKVDKRVEPKSCDKTHTCPPCPAPELLGGPSV |
| | modifications or | FLFPPKPKDTLMISRTPEVTCVVVDVSHEDPEVKFNWYVDGVEVHNAK |
| | mutations) | TKPREEQYNSTYRVVSVLTVLHQDWLNGKEYKCKVSNKALPAPIEKTI |
| | | SKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPG |
| 45 | Heavy chain variable | QVQLVQSGAEVKKPGASVKISCKASGYTFTDYYINWVRQAPGQGLEWI |
| | region | GWIYPGSVNTKYAQKFQGRATLTVDTSTSTAYIELSSLRSEDTAVYYC |
| | | ARYSNWFFDVWGQGTTVTVSS |
| 34 | HCDR1 (Kabat) | DYYIN |
| 43 | HCDR2 (Kabat) | WIYPGSVNTKYAQKFQG |
| 36 | HCDR3 (Kabat) | YSNWFFDV |

Humainzed 3H4 light chain 2 (h3H4-L2)

| | | |
|---|---|---|
| 46 | h3H4-L2, kappa | DIQMTQSPSSLSASVGDRVTITCRASQEISGYLSWLQQKPGKAPKLLI |
| | | YAASTLDSGVPSRFSGSGSGTDYTLTISSLQPEDFATYYCLQYASYPY |
| | | TFGQGTKLEIKRTVAAPSVFIFPPSDEQLKSGTASVVCLLNNFYPREA |
| | | KVQWKVDNALQSGNSQESVTEQDSKDSTYSLSSTLTLSKADYEKHKVY |
| | | ACEVTHQGLSSPVTKSFNRGEC |
| 47 | Light chain variable | DIQMTQSPSSLSASVGDRVTITCRASQEISGYLSWLQQKPGKAPKLLI |
| | region | YAASTLDSGVPSRFSGSGSGTDYTITISSLQPEDFATYYCLQYASYPY |
| | | TFGQGTKLEIK |
| 38 | LCDR1 (Kabat) | RASQEISGYIS |
| 39 | LCDR2 (Kabat) | AASTLDS |
| 40 | LCDR3 (Kabat) | LQYASYPYT |

Humainzed 3H4 light chain 3 (h3H4-L3)

| | | |
|---|---|---|
| 48 | h3H4-L3, kappa | DIQMTQSPSSLSASVGDRVTITCRASQEISGYLSWLQQKPGKAPKRLI |
| | | YAASTLDSGVPSRFSGSGSGTDYTLTISSLQPEDFATYYCLQYASYPY |
| | | TFGQGTKLEIKRTVAAPSVFIFPPSDEQLKSGTASVVCLLNNEYPREA |
| | | KVQWKVDNALQSGNSQESVTEQDSKDSTYSLSSTLTLSKADYEKHKVY |
| | | ACEVTHQGLSSPVTKSFNRGEC |
| 49 | Light chain variable | DIQMTQSPSSLSASVGDRVTITCRASQEISGYLSWLQQKPGKAPKRLI |
| | region | YAASTLDSGVPSRFSGSGSGTDYTLTISSLQPEDFATYYCLQYASYPY |
| | | TFGQGTKLEIK |
| 38 | LCDR1 (Kabat) | RASQEISGYLS |
| 39 | LCDR2 (Kabat) | AASTLDS |
| 40 | LCDR3 (Kabat) | LQYASYPYT |

Murine 2.3A9

| | | |
|---|---|---|
| 50 | Heavy chain variable | QVQLQQSDAELVKPGTSVKISCKASGYTFTDHAIHWLKQEPDQGLEWI |
| | region | GYISPGYGDIEYNEKEKGKATLTADKSSSTAYMQLSSLTSEDSAVYFC |
| | | KVTTGYWGQGTTLTVS |
| 51 | HCDR1 (Kabat) | DHAIH |
| 52 | HCDR2 (Kabat) | YISPGYGDIEYNEKFKG |
| 53 | HCDR3 (Kabat) | TTGY |
| 54 | Light chain variable | DVLMTQTPLTLSVTIGQPASISCKSSQSLLNSDGKTYLNWLLQRPGQS |
| | region | PKRLIYLVSELDSGVPDRFTGSGSGTDFTLKISRVEAEDLGIYYCWQG |
| | | THFPHTFGAGTKLELKR |
| 55 | LCDR1 (Kabat) | KSSQSLLNSDGKTYLN |
| 56 | LCDR2 (Kabat) | LVSELDS |
| 57 | LCDR3 (Kabat) | WQGTHFPHT |

Humainzed 2.3A9 heavy chain 2 (h2.3A9-H2)

| | | |
|---|---|---|
| 58 | h2.3A9-H2, IgG1 | QVQLVQSGAEVKKPGASVKVSCKASGYTFTDHAIHWLRQAPGQGLEWM |
| | (Fc or constant region | GYISPGYGDIEYAQKFQGRVTMTADTSTSTAYMELSSLRSEDTAVYYC |
| | may be of any IgG | KVTTGYWGQGTTVTVSSASTKGPSVFPLAPSSKSTSGGTAALGCLVKD |
| | isotype | YFPEPVTVSWNSGALTSGVHTFPAVLQSSGLYSLSSVVTVPSSSIGTQ |
| | and/or contain any | TYICNVNHKPSNTKVDKRVEPKSCDKTHTCPPCPAPELLGGPSVFLFP |
| | modifications or | PKPKDTLMISRTPEVTCVVVDVSHEDPEVKFNWYVDGVEVHNAKTKPR |
| | mutations) | EEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHY |
| | | TQKSLSLSPG |
| 59 | Heavy chain variable | QVQLVQSGAEVKKPGASVKVSCKASGYTFTDHAIHWLRQAPGQGLEWM |
| | region | GYISPGYGDIEYAQKFQGRVTMTADTSTSTAYMELSSLRSEDTAVYYC |
| | | KVTTGYWGQGTTVTVSS |
| 51 | HCDR1 (Kabat) | DHAIH |
| 60 | HCDR2 (Kabat) | YISPGYGDIEYAQKFQG |
| 53 | HCDR3 (Kabat) | TTGY |

Humainzed 2.3A9 heavy chain 3 (h2.3A9-H3)

| | | |
|---|---|---|
| 61 | h2.3A9-H3, IgG1 | QVQLVQSGAEVKKPGASVKISCKASGYTFTDHAIHWLRQAPGQGLEWI |
| | (Fc or constant region | GYISPGYGDIEYAQKFQGRATLTADTSTSTAYMELSSLRSEDTAVYYC |
| | may be of any IgG | KVTTGYWGQGTTLTVSSASTKGPSVFPLAPSSKSTSGGTAALGCLVKD |
| | isotype | YFPEPVTVSWNSGALTSGVHTFPAVLQSSGLYSLSSVVTVPSSSIGTQ |
| | and/or contain any | TYICNVNHKPSNTKVDKRVEPKSCDKTHTCPPCPAPELLGGPSVFLFP |
| | modifications or | PKPKDTLMISRTPEVTCVVVDVSHEDPEVKFNWYVDGVEVHNAKTKPR |
| | mutations) | EEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHY |
| | | TQKSLSLSPG |
| 62 | Heavy chain variable | QVQLVQSGAEVKKPGASVKISCKASGYTFTDHAIHWLRQAPGQGLEWI |
| | region | GYISPGYGDIEYAQKFQGRATITADTSTSTAYMELSSLRSEDTAVYYC |
| | | KVTTGYWGQGTTLTVSS |
| 51 | HCDR1 (Kabat) | DHAIH |
| 60 | HCDR2 (Kabat) | YISPGYGDIEYAQKFQG |
| 53 | HCDR3 (Kabat) | TTGY |

Humainzed 2.3A9 heavy chain 2b (h2.3A9-H2b)

| | | |
|---|---|---|
| 63 | h2.3A9-H2b, IgG1 | QVQLVQSGAEVKKPGASVKVSCKASGYTFTDHAIHWLRQAPGQGLEWM |
| | (Fc or constant region | GYISPGYGDIEYAQKFQGRVTMTADKSTSTAYMELSSLRSEDTAVYYC |
| | may be of any IgG | KVTTGYWGQGTTVTVSSASTKGPSVFPLAPSSKSTSGGTAALGCLVKD |
| | isotype | YFPEPVTVSWNSGALTSGVHTFPAVLQSSGLYSLSSVVTVPSSSIGTQ |
| | and/or contain any | TYICNVNHKPSNTKVDKRVEPKSCDKTHTCPPCPAPELLGGPSVFLFP |
| | modifications or | PKPKDTLMISRTPEVTCVVVDVSHEDPEVKFNWYVDGVEVHNAKTKPR |
| | mutations) | EEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHY |
| | | TQKSLSLSPG |
| 64 | Heavy chain variable | QVQLVQSGAEVKKPGASVKVSCKASGYTFTDHAIHWLRQAPGQGLEWM |
| | region | GYISPGYGDIEYAQKFQGRVTMTADKSTSTAYMELSSLRSEDTAVYYC |
| | | KVTTGYWGQGTTVTVSS |
| 51 | HCDR1 (Kabat) | DHAIH |
| 60 | HCDR2 (Kabat) | YISPGYGDIEYAQKFQG |
| 53 | HCDR3 (Kabat) | TTGY |

Humainzed 2.3A9 heavy chain 2 (h2.3A9-H3b)

| | | |
|---|---|---|
| 65 | h2.3A9-H3b, IgG1 | QVQLVQSGAEVKKPGASVKISCKASGYTFTDHAIHWLRQAPGQGLEWI |
| | (Fc or constant region | GYISPGYGDIEYAQKFQGRATLTADKSTSTAYMELSSLRSEDTAVYYC |
| | may be of any IgG | KVTTGYWGQGTTLTVSSASTKGPSVFPLAPSSKSTSGGTAALGCLVKD |
| | isotype | YFPEPVTVSWNSGALTSGVHTFPAVLQSSGLYSLSSVVTVPSSSLGTQ |
| | and/or contain any | TYICNVNHKPSNTKVDKRVEPKSCDKTHTCPPCPAPELLGGPSVFLFP |
| | modifications or | PKPKDTLMISRTPEVTCVVVDVSHEDPEVKFNWYVDGVEVHNAKTKPR |
| | mutations) | EEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHY |
| | | TQKSLSLSPG |
| 66 | Heavy chain variable | QVQLVQSGAEVKKPGASVKISCKASGYTETDHAIHWLRQAPGQGLEWI |
| | region | GYISPGYGDIEYAQKFQGRATLTADKSTSTAYMELSSLRSEDTAVYYC |
| | | KVTTGYWGQGTTLTVSS |
| 51 | HCDR1 (Kabat) | DHAIH |
| 60 | HCDR2 (Kabat) | YISPGYGDIEYAQKFQG |
| 53 | HCDR3 (Kabat) | TTGY |

Humainzed 2.3A9 light chain 2 (h2.3A9-L2)

| | | |
|---|---|---|
| 67 | h2.3A9-L2, kappa | DVVMTQSPLSLPVTLGQPASISCRSSQSLLNSDGKTYLNWLQQRPGQS |
| | | PRRLIYLVSELDSGVPDRFSGSGSGTDFTLKISRVEAEDVGVYYCWQG |
| | | THEPHTFGQGTKLEIKRTVAAPSVFIFPPSDEQLKSGTASVVCLLNNF |
| | | YPREAKVQWKVDNALQSGNSQESVTEQDSKDSTYSLSSTLTLSKADYE |
| | | KHKVYACEVTHQGLSSPVTKSFNRGEC |
| 68 | Light chain variable | DVVMTQSPLSLPVTLGQPASISCRSSQSLLNSDGKTYLNWLQQRPGQS |
| | region | PRRLIYLVSELDSGVPDRFSGSGSGTDFTLKISRVEAEDVGVYYCWQG |
| | | THEPHTFGQGTKLEIK |
| 55 | LCDR1 (Kabat) | RSSQSLLNSDGKTYLN |
| 56 | LCDR2 (Kabat) | LVSEEDS |
| 57 | LCDR3 (Kabat) | WQGTHFPHT |

Murine 20B3.1

| | | |
|---|---|---|
| 69 | Heavy chain variable | EVKLVESGGGLVKPGGSLKLSCAASGFTFSSFGMSWVRQTPEKRLEWV |
| | region | ATISGGGSNTYYPDSVKGRFTISRDNAKNNLYLQMTSLTSEDTALYYC |
| | | TRIYDVAWFAYWGQGTLVTVS |
| 70 | HCDR1 (Kabat) | SFGMS |
| 71 | HCDR2 (Kabat) | TISGGGSNTYYPDSVKG |
| 72 | HCDR3 (Kabat) | IYDVAWFAY |
| 73 | Light chain variable | DIVLTQSPASLAVSIGQRATISCRASESVDNSGISFMNWFQQKPGQPP |
| | region | KLLIYAASNQGSGVPARFSGSGSGTDFSLNIHPMEEDDTAMYFCQQSK |
| | | EVPWTFGGGTKLEIKR |
| 74 | LCDR1 (Kabat) | RASESVDNSGISFMN |
| 75 | LCDR2 (Kabat) | AASNQGS |
| 76 | LCDR3 (Kabat) | QQSKEVPWT |

Humainzed 20B3.1 heavy chain 2 (h20B3.1-H2)

| | | |
|---|---|---|
| 77 | h20B3.1-H2, IgG1 | EVQLVESGGGLVQPGGSLRISCAASGFTFSSFGMSWVRQAPGKGLEWV |
| | (Fc or constant region | ATISGGGSNTYYADSVKGRFTISRDNSKNTLYLQMNSLRAEDTAVYYC |
| | may be of any IgG | TRIYDVAWFAYWGQGTLVTVSSASTKGPSVFPLAPSSKSTSGGTAALG |
| | isotype | CLVKDYFPEPVTVSWNSGALTSGVHTFPAVLQSSGLYSLSSVVTVPSS |
| | and/or contain any | SLGTQTYICNVNHKPSNTKVDKRVEPKSCDKTHTCPPCPAPELLGGPS |
| | modifications or | VFLFPPKPKDTLMISRTPEVTCVVVDVSHEDPEVKFNWYVDGVEVHNA |
| | mutations) | KTKPREEQYNSTYRVVSVLTVLHQDWLNGKEYKCKVSNKALPAPIEKT |
| | | ISKAKGQPREPQVYTIPPSREEMTKNQVSLTCLVKGFYPSDIAVEWES |
| | | NGQPENNYKTTPPVLDSDGSEFLYSKLTVDKSRWQQGNVFSCSVMHEA |
| | | LHNHYTQKSLSLSPG |
| 78 | Heavy chain variable | EVQLVESGGGLVQPGGSLRLSCAASGFTFSSFGMSWVRQAPGKGLEWV |
| | region | ATISGGGSNTYYADSVKGRFTISRDNSKNTLYLQMNSLRAEDTAVYYC |
| | | TRIYDVAWFAYWGQGTLVTVSS |
| 70 | HCDR1 (Kabat) | SFGMS |
| 79 | HCDR2 (Kabat) | TISGGGSNTYYADSVKG |
| 72 | HCDR3 (Kabat) | IYDVAWFAY |

Humainzed 20B3.1 light chain 2 (h20B3.1-L2)

| | | |
|---|---|---|
| 80 | h20B3.1-L2, kappa | DIQMTQSPSSLSASVGDRVTITCRASESVDNSGISFMNWFQQKPGKAP |
| | | KLLIYAASNQGSGVPSRFSGSGSGTDFTLTISSLQPEDFATYYCQQSK |
| | | EVPWTFGQGTKLEIKRTVAAPSVFIFPPSDEQLKSGTASVVCLLNNFY |
| | | PREAKVQWKVDNALQSGNSQESVTEQDSKDSTYSLSSTLTLSKADYEK |
| | | HKVYACEVTHQGLSSPVTKSFNRGEC |
| 81 | Light chain variable | DIQMTQSPSSLSASVGDRVTITCRASESVDNSGISFMNWFQQKPGKAP |
| | region | KLLIYAASNQGSGVPSRFSGSGSGTDFTLTISSLQPEDFATYYCQQSK |
| | | EVPWTFGQGTKLEIK |
| 74 | LCDR1 (Kabat) | RASESVDNSGISFMN |
| 75 | LCDR2 (Kabat) | AASNQGS |
| 76 | LCDR3 (Kabat) | QQSKEVPWT |

Humainzed 20B3.1 light chain 3 (h20B3,1-L3)

| | | |
|---|---|---|
| 82 | h20B3.1-L3, kappa | DIQLTQSPSSLSASVGDRATITCRASESVDNSGISFMNWFQQKPGKAP |
| | | KLLIYAASNQGSGVPSRFSGSGSGTDFTLTISSMQPEDFATYYCQQSK |
| | | EVPWTFGQGTKLEIKRTVAAPSVFIFPPSDEQLKSGTASVVCLLNNFY |
| | | PREAKVQWKVDNALQSGNSQESVTEQDSKDSTYSLSSTLTLSKADYEK |
| | | HKVYACEVTHQGLSSPVTKSFNRGEC |
| 83 | Light chain variable | DIQLTQSPSSLSASVGDRATITCRASESVDNSGISFMNWEQQKPGKAP |
| | region | KLLIYAASNQGSGVPSRFSGSGSGTDFTLTISSMQPEDFATYYCQQSK |
| | | EVPWTFGQGTKLEIK |
| 74 | LCDR1 (Kabat) | RASESVDNSGISFMN |
| 75 | LCDR2 (Kabat) | AASNQGS |
| 76 | LCDR3 (Kabat) | QQSKEVPWT |

Murine 1B8

| | | |
|---|---|---|
| 84 | Heavy chain variable | EVQLVLSGGGFVQPGGSLKLSCAASGFTFSSYAMSWVRQNPERRLVWV |
| | region | ATITGGGRNTYYPDSVKGRFTISRDNAKNTLYLQMSSLRSEDTAMYYC |
| | | TRQGYDGYTWFAYWGQGTLVTVS |
| 85 | HCDR1 (Kabat) | SYAMS |
| 86 | HCDR2 (Kabat) | TITGGGRNTYYPDSVKG |
| 87 | HCDR3 (Kabat) | QGYDGYTWFAY |
| 88 | Light chain variable | DIVLTQSPTSLAVSLGQRATISCRASESVDNSGISFMNWFQQKPGQPP |
| | region | KLLIYAASNPGSGVPARFSGSGSGTDFSLNIHPMEEDDTAMYFCQQSK |
| | | EVPWTFGGGTELEIKR |
| 89 | LCDR1 (Kabat) | RASESVDNSGISFMN |
| 90 | LCDR2 (Kabat) | AASNPGS |
| 91 | LCDR3 (Kabat) | QQSKEVPWT |

Humainzed 1B8 heavy chain 2 (h1B8-H2)

| | | |
|---|---|---|
| 92 | h1B8-H2, IgG1 | EVQLVESGGGLVQPGGSLRLSCAASGFTFSSYAMSWVRQAPGKGLEWV |
| | (Fc or constant region | ATITGGGRNTYYADSVKGRFTISRDNSKNTLYLQMNSLRAEDTAVYYC |
| | may be of any IgG | TRQGYDGYTWFAYWGQGTLVTVSSASTKGPSVFPLAPSSKSTSGGTAA |
| | isotype | LGCLVKDYFPEPVTVSWNSGALTSGVHTFPAVLQSSGLYSLSSVVTVP |
| | and/or contain any | SSSLGTQTYICNVNHKPSNTKVDKRVEPKSCDKTHTCPPCPAPELLGG |
| | modifications or | PSVFLFPPKPKDTLMISRTPEVTCVVVDVSHEDPEVKFNWYVDGVEVH |
| | mutations) | NAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPG |
| 93 | Heavy chain variable | EVQLVESGGGLVQPGGSLRLSCAASGFTFSSYAMSWVRQAPGKGLEWV |
| | region | ATITGGGRNTYYADSVKGRFTISRDNSKNTLYLQMNSLRAEDTAVYYC |
| | | TRQGYDGYTWEAYWGQGTLVTVSS |
| 85 | HCDR1 (Kabat) | SYAMS |
| 94 | HCDR2 (Kabat) | TITGGGRNTYYADSVKG |
| 87 | HCDR3 (Kabat) | QGYDGYTWFAY |

Humainzed 1B8 light chain 2 (h1B8-L2)

| | | |
|---|---|---|
| 95 | h1B8-L2, kappa | DIQMTQSPSSLSASVGDRVTITCRASESVDNSGISFMNWFQQKPGKAP |
| | | KLLIYAASNPGSGVPSRFSGSGSGTDFTLTISSLQPEDFATYYCQQSK |
| | | EVPWTFGQGTKLEIKRTVAAPSVFIFPPSDEQLKSGTASVVCLLNNFY |
| | | PREAKVQWKVDNALQSGNSQESVTEQDSKDSTYSLSSTLTLSKADYEK |
| | | HKVYACEVTHQGLSSPVTKSFNRGEC |
| 96 | Light chain variable | DIQMTQSPSSLSASVGDRVTITCRASESVDNSGISFMNWFQQKPGKAP |
| | region | KLLIYAASNPGSGVPSRFSGSGSGTDFTLTISSLQPEDFATYYCQQSK |
| | | EVPWTFGQGTKLEIK |
| 89 | LCDR1 (Kabat) | RASESVDNSGISFMN |
| 90 | LCDR2 (Kabat) | AASNPGS |
| 91 | LCDR3 (Kabat) | QQSKEVPWT |

Humainzed 1B8 light chain 3 (h1B8-L3)

| | | |
|---|---|---|
| 97 | h1B8-L3, kappa | DIQLTQSPSSLSASVGDRATITCRASESVDNSGISFMNWFQQKPGKAP |
| | | KLLIYAASNPGSGVPSRFSGSGSGTDFTLTISSMQPEDFATYYCQQSK |
| | | EVPWTFGQGTKLEIKRTVAAPSVFIFPPSDEQLKSGTASVVCLLNNEY |
| | | PREAKVQWKVDNALQSGNSQESVTEQDSKDSTYSLSSTLTLSKADYEK |
| | | HKVYACEVTHQGLSSPVTKSFNRGEC |
| 98 | Light chain variable | DIQLTQSPSSLSASVGDRATITCRASESVDNSGISFMNWFQQKPGKAP |
| | region | KLLIYAASNPGSGVPSRFSGSGSGTDFTLTISSMQPEDFATYYCQQSK |
| | | EVPWTFGQGTKLEIK |
| 89 | LCDR1 (Kabat) | RASESVDNSGISFMN |
| 90 | LCDR2 (Kabat) | AASNPGS |
| 91 | LCDR3 (Kabat) | QQSKEVPWT |

TABLE 6
Other sequences

| SEQ | | |
|---|---|---|
| ID NO | Description | Sequence |
| 99 | Human immunoglobulin | ASTKGPSVFPLAPSSKSTSGGTAALGCLVKDYFPEPVTVSWNSGALTS |
| | G1 (IgG1) heavy chain | GVHTFPAVLQSSGLYSLSSVVTVPSSSIGTQTYICNVNHKPSNTKVDK |
| | constant (CH) region, | KVEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTVL |
| | | HQDWINGKEYKCKVSNKALPAPIEKTISKAKGQPREPQVYTLPPSRDE |
| | | LTKNQVSLTCLVKGFYPSDIAVEWESNGQPENNYKTTPPVLDSDGSFF |
| | | LYSKLTVDKSRWQQGNVFSCSVMHEALHNHYTQKSLSLSPG |
| 100 | Human immunoglobulin | ASTKGPSVFPLAPSSKSTSGGTAALGCLVKDYFPEPVTVSWNSGALTS |
| | G1 (IgG1) heavy chain | GVHTFPAVLQSSGLYSLSSVVTVPSSSIGTQTYICNVNHKPSNTKVDK |
| | constant (CH) region | RVEPKSCDKTHTCPPCPAPELLGGPSVELFPPKPKDTLMISRTPEVTC |
| | | VVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSREE |
| | | MTKNQVSLTCLVKGFYPSDIAVEWESNGQPENNYKTTPPVLDSDGSFF |
| | | LYSKLTVDKSRWQQGNVFSCSVMHEALHNHYTQKSLSLSPG |
| 101 | Modified IgG1 CH | ASTKGPSVFPLAPSSKSTSGGTAALGCLVKDYFPEPVTVSWNSGALTS |
| | region-X2 mutations | GVHTEPAVLQSSGLYSLSSVVTVPSSSLGTQTYICNVNHKPSNTKVDK |
| | | KVEPKSCDKTHTCPPCPAPELLQGPSVFLFPPKPKDTLMISRTPEVTC |
| | | VVVDVSDEDPEVKFNWYVDGVEVHNAKTKPREEQYNSTYRVVSVLTVL |
| | | HQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFF |
| | | LYSKLTVDKSRWQQGNVFSCSVMHEALHNHYTQKSLSLSPG |
| 102 | Modified IgG1 CH | ASTKGPSVFPLAPSSKSTSGGTAALGCLVKDYFPEPVTVSWNSGALTS |
| | region-X3 mutations | GVHTFPAVLQSSGLYSLSSVVTVPSSSIGTQTYICNVNHKPSNTKVDK |
| | | KVEPKSCDKTHTCPPCPAPELLGGPDVFLFPPKPKDTLMISRTPEVTC |
| | | VVVDVSDEDPEVKFNWYVDGVEVHNAKTKPREEQYNSTYRVVSVLTVL |
| | | HQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFF |
| | | LYSKLTVDKSRWQQGNVFSCSVMHEALHNHYTQKSLSLSPG |
| 103 | Modified IgG1 CH | ASTKGPSVFPLAPSSKSTSGGTAALGCLVKDYFPEPVTVSWNSGALTS |
| | region-X4 mutations | GVHTFPAVLQSSGLYSLSSVVTVPSSSIGTQTYICNVNHKPSNTKVDK |
| | | KVEPKSCDKTHTCPPCPAPELLGGPDVFLFPPKPKDTLMISRTPEVTC |
| | | VVVDVSDEDPEVKFNWYVDGVEVHNAKTKPREEQYNSTYRVVSVLTVL |
| | | HQDWLNGKEYKCKVSNKAYPAPEEKTISKAKGQPREPQVYTLPPSRDE |
| | | LTKNQVSLTCLVKGFYPSDIAVEWESNGQPENNYKTTPPVLDSDGSFF |
| | | LYSKLTVDKSRWQQGNVFSCSVMHEALHNHYTQKSLSLSPG |
| 104 | Modified IgG1 CH | ASTKGPSVFPLAPSSKSTSGGTAALGCLVKDYFPEPVTVSWNSGALTS |
| | region-V1 mutations | GVHTEPAVLQSSGLYSLSSVVTVPSSSLGTQTYICNVNHKPSNTKVDK |
| | | KVEPKSCDKTHTCPPCPAPELLGGDSVFLFPPKPKDTLMISRTPEVTC |
| | | VVVDVSHEDPEVKFNWYVDGVEVHNAKTKPREEQYNSTYRVVSVLTVL |
| | | HQDWLNGKEYKCKVSNKAEPAPIEKTISKAKGQPREPQVYTLPPSRDE |
| | | LTKNQVSLTCLVKGFYPSDIAVEWESNGQPENNYKTTPPVLDSDGSFF |
| | | LYSKLTVDKSRWQQGNVFSCSVMHEALHNHYTQKSLSLSPG |
| 105 | Modified IgG1 CH | ASTKGPSVFPLAPSSKSTSGGTAALGCLVKDYFPEPVTVSWNSGALTS |
| | region-V2 mutations | GVHTFPAVLQSSGLYSLSSVVTVPSSSIGTQTYICNVNHKPSNTKVDK |
| | | KVEPKSCDKTHTCPPCPAPELLGDPSVFLFPPKPKDTLMISRTPEVTC |
| | | VVVDVSHEDGEVKFNWYVDGVEVHNAKTKPREEQYNSTYRVVSVLTVL |
| | | HQDWLNGKEYKCKVSNKALPRPIEKTISKAKGQPREPQVYTLPPSRDE |
| | | LTKNQVSLTCLVKGFYPSDIAVEWESNGQPENNYKTTPPVLDSDGSFF |
| | | LYSKLTVDKSRWQQGNVFSCSVMHEALHNHYTQKSLSLSPG |
| 106 | Modified IgG1 CH | ASTKGPSVFPLAPSSKSTSGGTAALGCLVKDYFPEPVTVSWNSGALTS |
| | region-V3 mutations | GVHTFPAVLQSSGLYSLSSVVTVPSSSLGTQTYICNVNHKPSNTKVDK |
| | | KVEPKSCDKTHTCPPCPAPELLGDPSVFLFPPKPKDTLMISRTPEVTC |
| | | VVVDVSDEDGEVKFNWYVDGVEVHNAKTKPREEQYNSTYRVVSVLTVL |
| | | HQDWLNGKEYKCKVSNKALPRPIEKTISKAKGQPREPQVYTLPPSRDE |
| | | LTKNQVSLTCLVKGFYPSDIAVEWESNGQPENNYKTTPPVLDSDGSFE |
| | | LYSKLTVDKSRWQQGNVFSCSVMHEALHNHYTQKSLSLSPG |
| 107 | Modified IgG1 CH | ASTKGPSVFPLAPSSKSTSGGTAALGCLVKDYFPEPVTVSWNSGALTS |
| | region-V4 mutations | GVHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVTC |
| | | VVVDVSDEDPEVKFNWYVDGVEVHNAKTKPREEQYNSTYRVVSVLTVL |
| | | HQDWLNGKEYKCKVSNKAFPAPIEKTISKAKGQPREPQVYTLPPSRDE |
| | | LTKNQVSLTCLVKGFYPSDIAVEWESNGQPENNYKTTPPVLDSDGSFF |
| | | LYSKLTVDKSRWQQGNVFSCSVMHEALHNHYTQKSLSLSPG |
| 108 | Modified IgG1 CH | ASTKGPSVFPLAPSSKSTSGGTAALGCLVKDYFPEPVTVSWNSGALTS |
| | region-V5 mutations | GVHTFPAVLQSSGLYSLSSVVTVPSSSIGTQTYICNVNHKPSNTKVDK |
| | | KVEPKSCDKTHTCPPCPAPELLGGPDVFLFPPKPKDTIMISRTPEVTC |
| | | VVVDVEHEDPEVKFNWYVDGVEVHNAKTKPREEQYNSTYRVVSVLTVL |
| | | HQDWINGKEYKCKVSNKALPAPIEKTISKAKGQPREPQVYTLPPSRDE |
| | | LTKNQVSLTCLVKGFYPSDIAVEWESNGQPENNYKTTPPVLDSDGSFF |
| | | LYSKLTVDKSRWQQGNVFSCSVMHEALHNHYTQKSLSLSPG |
| 109 | Modified IgG1 CH | ASTKGPSVFPLAPSSKSTSGGTAALGCLVKDYFPEPVTVSWNSGALTS |
| | region-V6 mutations | GVHTFPAVLQSSGLYSLSSVVTVPSSSIGTQTYICNVNHKPSNTKVDK |
| | | KVEPKSCDKTHTCPPCPAPELLDGPSVFLFPPKPKDTLMISRTPEVTC |
| | | VVVDVEHEDPEVKFNWYVDGVEVHNAKTKPREEQYNSTYRVVSVLTVL |
| | | HQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFE |
| | | LYSKLTVDKSRWQQGNVFSCSVMHEALHNHYTQKSLSLSPG |
| 110 | Modified IgG1 CH | ASTKGPSVFPLAPSSKSTSGGTAALGCLVKDYFPEPVTVSWNSGALTS |
| | region-V12 mutations | GVHTFPAVLQSSGLYSLSSVVTVPSSSLGTQTYICNVNHKPSNTKVDK |
| | | KVEPKSCDKTHTCPPCPAPDLLGDPSVFLFPPKPKDTLMISRTPEVTC |
| | | VVVDVSDEDGEVKFNWYVDGVEVHNAKTKPREEQYNSTYRVVSVLTVL |
| | | HQDWLNGKEYKCKVSNKALPRPIEKTISKAKGQPREPQVYTLPPSRDE |
| | | LTKNQVSLTCLVKGFYPSDIAVEWESNGQPENNYKTTPPVLDSDGSFF |
| | | LYSKLTVDKSRWQQGNVFSCSVMHEALHNHYTQKSLSLSPG |
| 111 | Human immunoglobulin | RTVAAPSVFIFPPSDEQLKSGTASVVCLLNNFYPREAKVQWKVDNALQ |
| | kappa constant region | SGNSQESVTEQDSKDSTYSLSSTLTLSKADYEKHKVYACEVTHQGLSS |
| | | PVTKSFNRGEC |

### Incorporation by Reference

Unless stated to the contrary, the entire disclosure of each of the patent documents and scientific articles referred to herein is incorporated by reference for all purposes.

### EQUIVALENTS/OTHER EMBODIMENTS

Those skilled in the art will recognize or be able to ascertain using no more than routine experimentation, many equivalents to the specific embodiments described herein. Such equivalents are intended to be encompassed by the following claims.

What is claimed is:

1. An antibody or an antigen-binding fragment thereof that is capable of binding to PD-1, wherein the antibody or antigen binding fragment thereof
(a) is a PD-1 agonist;
(b) has enhanced binding to an Fc receptor; and/or
(c) is non-fucosylated.

2. The antibody or antigen binding fragment thereof of claim 1, wherein the Fc receptor is an inhibitory Fc receptor (e.g., FcγRII).

3. The antibody or antigen binding fragment thereof of claim 1, wherein the Fc receptor is an activating Fc receptor (e.g., FcγRI or FcγRIII).

4. The antibody or antigen binding fragment thereof of claims 1-3, wherein the antibody or antigen binding fragment thereof has enhanced effector function.

5. The antibody or antigen binding fragment thereof of claim 4, wherein the effector function is antibody-dependent cellular cytotoxicity (ADCC), antibody-dependent Cellular phagocytosis (ADCP), and complement dependent cytotoxicity (CDC).

6. The antibody or antigen binding fragment thereof of any one of claims 1-5, wherein the antibody or antigen binding fragment thereof is capable of depleting a cell.

7. The antibody or antigen binding fragment thereof of claim 6, wherein the cell is a PD-1 expressing cell.

8. The antibody or antigen binding fragment thereof of claim 6 or 7, wherein the cell is an immune cell.

9. The antibody or antigen binding fragment thereof of claim 8, wherein the immune cell is a T cell, a B cell, a macrophage, a natural killer (NK) cell, a dendritic cell (DC), a monocyte, a neutrophil, a fibroblast, or an epithelial cell.

10. The antibody or antigen binding fragment thereof of claim 9, wherein the immune cell is a T cell or a B cell.

11. The antibody or antigen binding fragment thereof of any one of claims 8-10, wherein the immune cell is activated.

12. The antibody or antigen binding fragment thereof of claim 11, wherein the immune cell is an antigen-activated T cell or B cell.

13. The antibody or antigen binding fragment thereof of any one of claims 1-12, wherein the antibody or antigen binding fragment thereof is at least about 60%, about 75%, or about 90% non-fucosylated.

14. The antibody or antigen-binding fragment thereof of any one of claims 1-13, comprising:
(a) (i) a heavy chain variable (VH) region comprising:
heavy chain complementarity-determining region 1 (HCDR1) having the amino acid sequence of SEQ ID NO: 2, or a sequence differing in 1 or 2 amino acids therefrom,
HCDR2 having the amino acid sequence of SEQ ID NO: 3, or a sequence differing in 1 or 2 amino acids therefrom, and
HCDR3 having the amino acid sequence of SEQ ID NO: 4, or a sequence differing in 1 or 2 amino acids therefrom, and
(ii) a light chain variable (VL) region comprising:
light chain complementarity-determining region 1 (LCDR1) having the amino acid sequence of SEQ ID NO: 6, or a sequence differing in 1 or 2 amino acids therefrom,
LCDR2 having the amino acid sequence of SEQ ID NO: 7, or a sequence differing in 1 or 2 amino acids therefrom, and
LCDR3 having the amino acid sequence of SEQ ID NO: 8, or a sequence differing in 1 or 2 amino acids therefrom;
(b) (i) a VH region comprising:
HCDR1 having the amino acid sequence of SEQ ID NO: 10, or a sequence differing in 1 or 2 amino acids therefrom,
HCDR2 having the amino acid sequence of SEQ ID NO: 11, or a sequence differing in 1 or 2 amino acids therefrom, and
HCDR3 having the amino acid sequence of SEQ ID NO: 12, or a sequence differing in 1 or 2 amino acids therefrom; and
(ii) a VL region comprising:
LCDR1 having the amino acid sequence of SEQ ID NO: 14, or a sequence differing in 1 or 2 amino acids therefrom,
LCDR2 having the amino acid sequence of SEQ ID NO: 15, or a sequence differing in 1 or 2 amino acids therefrom, and
LCDR3 having the amino acid sequence of SEQ ID NO: 16, or a sequence differing in 1 or 2 amino acids therefrom,
(c) (i) a VH region comprising:
HCDR1 having the amino acid sequence of SEQ ID NO: 18, or a sequence differing in 1 or 2 amino acids therefrom,
HCDR2 having the amino acid sequence of SEQ ID NO: 19, or a sequence differing in 1 or 2 amino acids therefrom, and
HCDR3 having the amino acid sequence of SEQ ID NO: 20, or a sequence differing in 1 or 2 amino acids therefrom; and
(ii) a VL region comprising:
LCDR1 having the amino acid sequence of SEQ ID NO: 22, or a sequence differing in 1 or 2 amino acids therefrom,
LCDR2 having the amino acid sequence of SEQ ID NO: 23, or a sequence differing in 1 or 2 amino acids therefrom, and
LCDR3 having the amino acid sequence of SEQ ID NO: 24, or a sequence differing in 1 or 2 amino acids therefrom;
(d) (i) a VH region comprising:
HCDR1 having the amino acid sequence of SEQ ID NO: 26, or a sequence differing in 1 or 2 amino acids therefrom,
HCDR2 having the amino acid sequence of SEQ ID NO: 27, or a sequence differing in 1 or 2 amino acids therefrom, and
HCDR3 having the amino acid sequence of SEQ ID NO: 28, or a sequence differing in 1 or 2 amino acids therefrom; and
(ii) a VL region comprising:
LCDR1 having the amino acid sequence of SEQ ID NO: 30, or a sequence differing in 1 or 2 amino acids therefrom,
LCDR2 having the amino acid sequence of SEQ ID NO: 31, or a sequence differing in 1 or 2 amino acids therefrom, and
LCDR3 having the amino acid sequence of SEQ ID NO: 32, or a sequence differing in 1 or 2 amino acids therefrom;
(e) (i) a VH region comprising:
HCDR1 having the amino acid sequence of SEQ ID NO: 34, or a sequence differing in 1 or 2 amino acids therefrom,
HCDR2 having the amino acid sequence of SEQ ID NO: 35, or a sequence differing in 1 or 2 amino acids therefrom, and
HCDR3 having the amino acid sequence of SEQ ID NO: 36, or a sequence differing in 1 or 2 amino acids therefrom; and
(ii) a VL region comprising:
LCDR1 having the amino acid sequence of SEQ ID NO: 38, or a sequence differing in 1 or 2 amino acids therefrom,
LCDR2 having the amino acid sequence of SEQ ID NO: 39, or a sequence differing in 1 or 2 amino acids therefrom, and
LCDR3 having the amino acid sequence of SEQ ID NO: 40, or a sequence differing in 1 or 2 amino acids therefrom;
(f) (i) a VH region comprising:
HCDR1 having the amino acid sequence of SEQ ID NO: 34, or a sequence differing in 1 or 2 amino acids therefrom,
HCDR2 having the amino acid sequence of SEQ ID NO: 43, or a sequence differing in 1 or 2 amino acids therefrom, and
HCDR3 having the amino acid sequence of SEQ ID NO: 36, or a sequence differing in 1 or 2 amino acids therefrom; and
(ii) a VL region comprising:
LCDR1 having the amino acid sequence of SEQ ID NO: 38, or a sequence differing in 1 or 2 amino acids therefrom,
LCDR2 having the amino acid sequence of SEQ ID NO: 39, or a sequence differing in 1 or 2 amino acids therefrom, and
LCDR3 having the amino acid sequence of SEQ ID NO: 40, or a sequence differing in 1 or 2 amino acids therefrom;
(g) (i) a VH region comprising:
HCDR1 having the amino acid sequence of SEQ ID NO: 51, or a sequence differing in 1 or 2 amino acids therefrom,
HCDR2 having the amino acid sequence of SEQ ID NO: 52, or a sequence differing in 1 or 2 amino acids therefrom, and
HCDR3 having the amino acid sequence of SEQ ID NO: 53, or a sequence differing in 1 or 2 amino acids therefrom; and
(ii) a VL region comprising:
LCDR1 having the amino acid sequence of SEQ ID NO: 55, or a sequence differing in 1 or 2 amino acids therefrom,
LCDR2 having the amino acid sequence of SEQ ID NO: 56, or a sequence differing in 1 or 2 amino acids therefrom, and
LCDR3 having the amino acid sequence of SEQ ID NO: 57, or a sequence differing in 1 or 2 amino acids therefrom;
(h) (i) a VH region comprising:
HCDR1 having the amino acid sequence of SEQ ID NO: 51, or a sequence differing in 1 or 2 amino acids therefrom,
HCDR2 having the amino acid sequence of SEQ ID NO: 60, or a sequence differing in 1 or 2 amino acids therefrom, and
HCDR3 having the amino acid sequence of SEQ ID NO: 53, or a sequence differing in 1 or 2 amino acids therefrom; and
(ii) a VL region comprising:
LCDR1 having the amino acid sequence of SEQ ID NO: 55, or a sequence differing in 1 or 2 amino acids therefrom,
LCDR2 having the amino acid sequence of SEQ ID NO: 56, or a sequence differing in 1 or 2 amino acids therefrom, and
LCDR3 having the amino acid sequence of SEQ ID NO: 57, or a sequence differing in 1 or 2 amino acids therefrom,
(i) (i) a VH region comprising:
HCDR1 having the amino acid sequence of SEQ ID NO: 70, or a sequence differing in 1 or 2 amino acids therefrom,
HCDR2 having the amino acid sequence of SEQ ID NO: 71, or a sequence differing in 1 or 2 amino acids therefrom, and
HCDR3 having the amino acid sequence of SEQ ID NO: 72, or a sequence differing in 1 or 2 amino acids therefrom; and
(ii) a VL region comprising:
LCDR1 having the amino acid sequence of SEQ ID NO: 74, or a sequence differing in 1 or 2 amino acids therefrom,
LCDR2 having the amino acid sequence of SEQ ID NO: 75, or a sequence differing in 1 or 2 amino acids therefrom, and
LCDR3 having the amino acid sequence of SEQ ID NO: 76, or a sequence differing in 1 or 2 amino acids therefrom;
(j) (i) a VH region comprising:
HCDR1 having the amino acid sequence of SEQ ID NO: 70, or a sequence differing in 1 or 2 amino acids therefrom,
HCDR2 having the amino acid sequence of SEQ ID NO: 79, or a sequence differing in 1 or 2 amino acids therefrom, and
HCDR3 having the amino acid sequence of SEQ ID NO: 72, or a sequence differing in 1 or 2 amino acids therefrom; and
(ii) a VL region comprising:
LCDR1 having the amino acid sequence of SEQ ID NO: 74, or a sequence differing in 1 or 2 amino acids therefrom,
LCDR2 having the amino acid sequence of SEQ ID NO: 75, or a sequence differing in 1 or 2 amino acids therefrom, and
LCDR3 having the amino acid sequence of SEQ ID NO: 76, or a sequence differing in 1 or 2 amino acids therefrom;
(k) (i) a VH region comprising:
HCDR1 having the amino acid sequence of SEQ ID NO: 85, or a sequence differing in 1 or 2 amino acids therefrom,
HCDR2 having the amino acid sequence of SEQ ID NO: 86, or a sequence differing in 1 or 2 amino acids therefrom, and
HCDR3 having the amino acid sequence of SEQ ID NO: 87, or a sequence differing in 1 or 2 amino acids therefrom; and
(ii) a VL region comprising:
LCDR1 having the amino acid sequence of SEQ ID NO: 89, or a sequence differing in 1 or 2 amino acids therefrom,
LCDR2 having the amino acid sequence of SEQ ID NO: 90, or a sequence differing in 1 or 2 amino acids therefrom, and
LCDR3 having the amino acid sequence of SEQ ID NO: 91, or a sequence differing in 1 or 2 amino acids therefrom;
or
(l) (i) a VH region comprising:
HCDR1 having the amino acid sequence of SEQ ID NCO: 85, or a sequence differing in 1 or 2 amino acids therefrom,
HCDR2 having the amino acid sequence of SEQ ID NO: 94, or a sequence differing in 1 or 2 amino acids therefrom, and
HCDR3 having the amino acid sequence of SEQ ID NO: 87, or a sequence differing in 1 or 2 amino acids therefrom; and
(ii) a VL region comprising:
LCDR1 having the amino acid sequence of SEQ ID NO: 89, or a sequence differing in 1 or 2 amino acids therefrom,
LCDR2 having the amino acid sequence of SEQ ID NO: 90, or a sequence differing in 1 or 2 amino acids therefrom, and
LCDR3 having the amino acid sequence of SEQ ID NO: 91, or a sequence differing in 1 or 2 amino acids therefrom.

14. The antibody or antigen-binding fragment thereof of claim 13, wherein the HCDR1, HCDR2, HCDR3, LCDR1, LCDR2, and LCDR3 comprise amino acid sequences that collectively differ by no more than two amino acid residues from the sequences of;
(a) SEQ ID NO: 2, SEQ ID NO: 3, SEQ ID NO: 4, SEQ ID NO: 6, SEQ ID NO: 7, and SEQ ID NO: 8, respectively;
(b) SEQ ID NO: 10, SEQ ID NO: 11, SEQ ID NO: 12, SEQ ID NO: 14, SEQ ID NO: 15, and SEQ ID NO: 16, respectively;
(c) SEQ ID NO: 18, SEQ ID NO: 19, SEQ ID NO: 20, SEQ ID NO: 22, SEQ ID NO: 23, and SEQ ID NO: 24, respectively;
(d) SEQ ID NO: 26, SEQ ID NO 27, SEQ ID NO: 28, SEQ ID NO: 30, SEQ ID NO: 31, and SEQ ID NO: 32, respectively;
(e) SEQ ID NO: 34, SEQ ID NO: 35, SEQ ID NO: 36, SEQ ID NO: 38, SEQ ID NO: 39, and SEQ ID NO: 40, respectively;
(f) SEQ ID NO: 34, SEQ ID NO: 43, SEQ ID NO: 36, SEQ ID NO: 38, SEQ ID NO: 39, and SEQ ID NO: 40, respectively;
(g) SEQ ID NO: 51, SEQ ID NO: 52, SEQ ID NO: 53, SEQ ID NO: 55, SEQ ID NO: 56, and SEQ ID NO: 57, respectively;
(b) SEQ ID NO: 51, SEQ ID NO: 60, SEQ ID NO: 53, SEQ ID NO: 55, SEQ ID NO: 56, and SEQ ID NO: 57, respectively,
(i) SEQ ID NO: 70, SEQ ID NO: 71, SEQ ID NO: 72, SEQ ID NO: 74, SEQ ID NO: 75, and SEQ ID NO: 76, respectively;
(j) SEQ ID NO: 70, SEQ ID NO: 79, SEQ ID NO: 72, SEQ ID NO: 74, SEQ ID NO: 75, and SEQ ID NO: 76, respectively;
(k) SEQ ID NO: 85, SEQ ID NO: 86, SEQ ID NO: 87, SEQ ID NO: 89, SEQ ID NO: 90, and SEQ ID NO: 91, respectively; or
(l) SEQ ID NO: 85, SEQ ID NO. 94, SEQ ID NO: 87, SEQ ID NO: 89, SEQ IID NO: 90, and SEQ ID NO: 91, respectively.

16. The antibody or antigen-binding fragment thereof of claim 15, comprising
(a) (i) a VH region comprising:
CDR-H1 comprising the amino acid sequence of SEQ ID NO: 2,
CDR-H2 comprising the amino acid sequence of SEQ ID NO: 3, and
CDR-H3 comprising the amino acid sequence of SEQ ID NO: 4; and
(ii) a VL region comprising:
CDR-L1 comprising the amino acid sequence of SEQ ID NO: 6,
CDR-L2 comprising the amino acid sequence of SEQ ID NO: 7, and
CDR-L3 comprising the amino acid sequence of SEQ ID NO: 8;
(b) (i) a VH region comprising:
CDR-H1 comprising the amino acid sequence of SEQ ID NO: 10,
CDR-H2 comprising the amino acid sequence of SEQ ID NO: 11, and
CDR-H3 comprising the amino acid sequence of SEQ ID NO: 12; and
(ii) a VL region comprising:
CDR-L1 comprising the amino acid sequence of SEQ ID NO: 14,
CDR-L2 comprising the amino acid sequence of SEQ ID NO: 15, and
CDR-L3 comprising the amino acid sequence of SEQ ID NO: 16;
(c) (i) a VII region comprising:
CDR-H1 comprising the amino acid sequence of SEQ ID NO: 18,
CDR-H2 comprising the amino acid sequence of SEQ ID NO: 19, and
CDR-H3 comprising the amino acid sequence of SEQ ID NO: 20, and
(ii) a VL region comprising:
CDR-L1 comprising the amino acid sequence of SEQ ID NO: 22,
CDR-L2 comprising the amino acid sequence of SEQ ID NO: 23, and
CDR-L3 comprising the amino acid sequence of SEQ ID NO: 24;
(d) (i) a VH region comprising:
CDR-H1 comprising the amino acid sequence of SEQ ID NO: 26,
CDR-H2 comprising the amino acid sequence of SEQ ID NO: 27, and
CDR-H3 comprising the amino acid sequence of SEQ ID NO: 28; and
(ii) a VL region comprising:
CDR-L1 comprising the amino acid sequence of SEQ ID NO: 30,
CDR-L2 comprising the amino acid sequence of SEQ ID NO: 31, and
CDR-L3 comprising the amino acid sequence of SEQ ID NO: 32;
(e) (i) a VH region comprising:
CDR-H1 comprising the amino acid sequence of SEQ ID NO: 34,
CDR-H2 comprising the amino acid sequence of SEQ ID NO: 35, and
CDR-H3 comprising the amino acid sequence of SEQ ID NO: 36; and
(ii) a VL region comprising:
CDR-L1 comprising the amino acid sequence of SEQ ID NO: 38,
CDR-L2 comprising the amino acid sequence of SEQ ID NO: 39, and
CDR-L3 comprising the amino acid sequence of SEQ ID NO: 40;
(f) (i) a VH region comprising:
CDR-H1 comprising the amino acid sequence of SEQ ID NO: 34,
CDR-H2 comprising the amino acid sequence of SEQ ID NO: 43, and
CDR1-H3 comprising the amino acid sequence of SEQ ID NO: 36; and
(ii) a VL region comprising:
CDR-L1 comprising the amino acid sequence of SEQ ID NO: 38,
CDR-L2 comprising the amino acid sequence of SEQ ID NO: 39, and
CDR-L3 comprising the amino acid sequence of SEQ ID NO: 40;
(g) (i) a VH region comprising:
CDR-H1 comprising the amino acid sequence of SEQ ID NO: 51,
CDR-H2 comprising the amino acid sequence of SEQ ID NO: 52, and
CDR-H3 comprising the amino acid sequence of SEQ ID NO: 53; and
(ii) a NL region comprising:
CDR-L1 comprising the amino acid sequence of SEQ ID NO: 55,
CDR-L2 comprising the amino acid sequence of SEQ ID NO: 56, and
CDR-L3 comprising the amino acid sequence of SEQ ID NCO: 57;
(h) (i) a VH region comprising:
CDR-H1 comprising the amino acid sequence of SEQ ID NO: 51,
CDR-H2 comprising the amino acid sequence of SEQ ID NO: 60, and
CDR-H3 comprising the amino acid sequence of SEQ ID NO: 53, and
(ii) a VL region comprising:
CDR-L1 comprising the amino acid sequence of SEQ ID NO: 55,
CDR-L2 comprising the amino acid sequence of SEQ ID NO: 56, and
CDR-L3 comprising the amino acid sequence of SEQ ID NO: 57;
(i) (i) a VH region comprising:
CDR-H1 comprising the amino acid sequence of SEQ ID NO: 70,
CDR-H2 comprising the amino acid sequence of SEQ ID NO: 71, and
CDR-H3 comprising the amino acid sequence of SEQ ID NO: 72; and
(ii) a VL, region comprising:
CDR-L1 comprising the amino acid sequence of SEQ ID NO: 74,
CDR-L2 comprising the amino acid sequence of SEQ ID NO: 75, and
CDR-L3 comprising the amino acid sequence of SEQ ID NO: 76;
(j) (i) a VH region comprising:
CDR-H1 comprising the amino acid sequence of SEQ ID NO: 70,
CDR-H2 comprising the amino acid sequence of SEQ ID NO: 79, and
CDR-H3 comprising the amino acid sequence of SEQ ID NO: 72; and
(ii) a VL region comprising:
CDR-L1 comprising the amino acid sequence of SEQ ID NO: 74,
CDR-L2 comprising the amino acid sequence of SEQ ID NO: 75, and
CDR-L3 comprising the amino acid sequence of SEQ ID NO: 76;
(k) (i) a VH region comprising:
CDR-H1 comprising the amino acid sequence of SEQ ID NO: 85,
CDR-H2 comprising the amino acid sequence of SEQ ID NO: 86, and
CDR-H3 comprising the amino acid sequence of SEQ ID NO: 87; and
(ii) a VL region comprising:
CDR-L1 comprising the amino acid sequence of SEQ ID NO: 89,
CDR-L2 comprising the amino acid sequence of SEQ ID NO: 90, and
CDR-L3 comprising the amino acid sequence of SEQ ID NO: 91;
or
(l) (i) a VH region comprising:
CDR-H1 comprising the amino acid sequence of SEQ ID NO: 85,
CDR-H2 comprising the amino acid sequence of SEQ ID NO: 86, and
CDR-H3 comprising the amino acid sequence of SEQ ID NO: 87; and
(ii) a VL region comprising:
CDR-L1 comprising the amino acid sequence of SEQ ID NO: 89,
CDR-L2 comprising the amino acid sequence of SEQ ID NO: 90, and
CDR-L3 comprising the amino acid sequence of SEQ ID NO: 91.

17. The antibody or antigen-binding fragment thereof of claim 16, comprising:
(i) a VH region comprising:
CDR-H11 comprising the amino acid sequence of SEQ ID NO: 34,
CDR-H2 comprising the amino acid sequence of SEQ ID NO: 43, and
CDR-H3 comprising the amino acid sequence of SEQ ID NO: 36; and
(ii) a VL region comprising:
CDR-L1 comprising the amino acid sequence of SEQ ID NO: 38,
CDR-L2 comprising the amino acid sequence of SEQ ID NO: 39, and
CDR-L3 comprising the amino acid sequence of SEQ ID NO: 40;

18. The antibody or antigen-binding fragment thereof of any one of claims 14-17, comprising
(a) a VH region comprising an amino acid sequence having at least 70%, 80%, 90%, 95%, 96%, 97%, 98%, or 99% sequence identity to the amino acid sequence of SEQ ID NO: 1 and a VL region comprising an amino acid sequence having at least 70%, 80%, 90%, 95%, 96%, 97%, 98%, or 99% sequence identity to the amino acid sequence of SEQ ID NO: 5;
(b) a VH region comprising an amino acid sequence having at least 70%, 80%, 90%, 95%, 96%, 97%, 98%, or 99% sequence identity to the amino acid sequence of SEQ ID NO: 9 and a VL region comprising an amino acid sequence having at least 70%, 80%, 90%, 95%, 96%, 97%, 98%, or 99% sequence identity to the amino acid sequence of SEQ ID NO: 13;
(c) a VH region comprising an amino acid sequence having at least 70%, 80%, 90%, 95%, 96%, 97%, 98%, or 99% sequence identity to the amino acid sequence of SEQ ID NO: 17 and a VL region comprising an amino acid sequence having at least 70%, 80%, 90%, 95%, 96%, 97%, 98%, or 99% sequence identity to the amino acid sequence of SEQ ID NO: 21;
(d) a VH region comprising an amino acid sequence having at least 70%, 80%, 90%, 95%, 96%, 97%, 98%, or 99% sequence identity to the amino acid sequence of SEQ ID NO: 25 and a VL region comprising an amino acid sequence having at least 70%, 80%, 90%, 95%, 96%, 97%, 98%, or 99% sequence identity to the amino acid sequence of SEQ ID NO: 29;
(e) a VH region comprising an amino acid sequence having at least 70%, 80%, 90%, 95%, 96%, 97%, 98%, or 99% sequence identity to the amino acid sequence of SEQ ID NO: 33 and a VL region comprising an amino acid sequence having at least 70%, 80%, 90%, 95%, 96%, 97%, 98%, or 99% sequence identity to the amino acid sequence of SEQ ID NO: 37;
(f) a VTI region comprising an amino acid sequence having at least 70%, 80%, 90%, 95%, 96%, 97%, 98%, or 99% sequence identity to the amino acid sequence of SEQ ID NO: 42 or 45 and a VL region comprising an amino acid sequence having at least 70%, 80%, 90%, 95%, 96%, 97%, 98%, or 99% sequence identity to the amino acid sequence of SEQ ID NO: 47 or 49;
(g) a VH region comprising an amino acid sequence having at least 70%, 80%, 90%, 95%, 96%, 97%, 98%, or 99% sequence identity to the amino acid sequence of SEQ ID NO: 50 and a VL region comprising an amino acid sequence having at least 70%, 80%, 90%, 95%, 96%, 97%, 98%, or 99% sequence identity to the amino acid sequence of SEQ ID NO: 54;
(h) a VH region comprising an amino acid sequence having at least 70%, 80%, 90%, 95%, 96%, 97%, 98%, or 99% sequence identity to the amino acid sequence of SEQ ID NO: 56, 62, 64, or 66 and a VL region comprising an amino acid sequence having at least 70%, 80%, 90%, 95%, 96%, 97%, 98%, or 99% sequence identity to the amino acid sequence of SEQ ID NO: 68;
(i) a VH region comprising an amino acid sequence having at least 70%, 80%, 90%, 95%, 96%, 97%, 98%, or 99% sequence identity to the amino acid sequence of SEQ ID NO: 69 and a VL region comprising an amino acid sequence having at least 70%, 80%, 90%, 95%, 96%, 97%, 8%, or 99% sequence identity to the amino acid sequence of SEQ ID NO: 73;
(j) a VH region comprising an amino acid sequence having at least 70%, 80%, 90%, 95%, 96%, 97%, 98%, or 99% sequence identity to the amino acid sequence of SEQ ID NO: 78 and a VL region comprising an amino acid sequence having at least 70%, 80%, 90%, 95%, 96%, 97%, 98%, or 99% sequence identity to the amino acid sequence of SEQ ID NO: 81 or 83;
(k) a VH region comprising an amino acid sequence having at least 70%, 80%, 90%, 95%, 96%, 97%, 98%, or 99% sequence identity to the amino acid sequence of SEQ ID NO: 84 and a VL region comprising an amino acid sequence having at least 70%, 80%, 90%, 95%, 96%, 97%, 98%, or 99% sequence identity to the amino acid sequence of SEQ ID NO: 88;
or
(l) a VH region comprising an amino acid sequence having at least 70%, 80%, 90%, 95%, 96%, 97%, 98%, or 99% sequence identity to the amino acid sequence of SEQ ID NO: 93 and a VL region comprising an amino acid sequence having at least 70%, 80%, 90%, 95%, 96%, 97%, 98%, or 99% sequence identity to the amino acid sequence of SEQ ID NO: 96 or 98.

19. The antibody or antigen-binding fragment thereof of claim 18, comprising:
(a) a VII region having the amino acid sequence of SEQ ID NO: 1 and a VL region having the amino acid sequence of the amino acid sequence of SEQ ID NO: 5,
(b) a VH region having the amino acid sequence of the amino acid sequence of SEQ FD NO: 9 and a VL region having the amino acid sequence of the amino acid sequence of SEQ ID NO: 13;
(c) a VH region having the amino acid sequence of the amino acid sequence of SEQ ID NO: 17 and a VL region having the amino acid sequence of the amino acid sequence of SEQ ID NO: 21,
(d) a VH region having the amino acid sequence of the amino acid sequence of SEQ ID NO. 25 and a VL region having the amino acid sequence of the amino acid sequence of SEQ ID NO: 29;
(e) a VH region having the amino acid sequence of the amino acid sequence of SEQ ID NO: 33 and a VL region having the amino acid sequence of the amino acid sequence of SEQ ID NO: 37;
(f) a VH region having the amino acid sequence of the amino acid sequence of SEQ ID NO: 42 or 45 and a VL region having the amino acid sequence of the amino acid sequence of SEQ ID NO: 47 or 49;
(g) a VH region having the amino acid sequence of the amino acid sequence of SEQ ID NO: 50 and a VL region having the amino acid sequence of the amino acid sequence of SEQ ID NO: 54,
(h) a VH region having the amino acid sequence of the amino acid sequence of SEQ ID NO: 56, 62, 64, or 66 and a VL region having the amino acid sequence of the amino acid sequence of SEQ ID NO: 68;
(i) a VH region having the amino acid sequence of the amino acid sequence of SEQ ID NO: 69 and a VL region having the amino acid sequence of the amino acid sequence of SEQ ID NO: 73;
(j) a VH region having the amino acid sequence of the amino acid sequence of SEQ ID NO: 78 and a VL region having the amino acid sequence of the amino acid sequence of SEQ ID NO: 81 or 83;
(k) a VH region having the amino acid sequence of the amino acid sequence of SEQ ID NO: 84 and a VL region having the amino acid sequence of the amino acid sequence of SEQ ID NO: 88;
or
(l) a VH region having the amino acid sequence of the amino acid sequence of SEQ ID NO: 93 and a VL region having the amino acid sequence of the amino acid sequence of SEQ ID NO: 96 or 98.

20. The antibody or antigen-binding fragment thereof of claim 19, comprising
a VH region having the amino acid sequence of the amino acid sequence of SEQ ID NO: 42 or 45 and a VL region having the amino acid sequence of the amino acid sequence of SEQ ID NO: 47 or 49.

21. The antibody or antigen binding fragment thereof of any one of claims 1-20, wherein the antibody is a humanized anti body.

22. The antibody or antigen binding fragment thereof of any one of claims 1-21, wherein the antigen binding fragment is a Fab, a Fab′ a Fab2, a F(ab′)2, Fv, a single-chain Fv (scFv), or a diabody.

23. The antibody or antigen binding fragment thereof of claim 22, wherein the antigen binding fragment is an scFv.

24. The antibody or antigen binding fragment thereof of any one of claims 1-23, wherein the antibody or antigen binding fragment thereof comprises an antibody heavy chain constant region.

25. The antibody or antigen binding fragment thereof of claim 24, wherein the antibody heavy chain constant region is a human IgG heavy chain constant region.

26. The antibody or antigen binding fragment thereof of claim 25, wherein the anti body heavy chain constant region is a human IgG1 heavy chain constant region.

27. The antibody or antigen binding fragment thereof of claim 26, wherein the antibody heavy chain constant region comprises an amino acid sequence having at least 70%, 80%, 90%, 95%, 96%, 97%, 98%, or 99% sequence identity to the amino acid sequence of SEQ ID NO: 100.

28. The antibody or antigen binding fragment thereof of claim 27, wherein the antibody heavy chain constant region has the amino acid sequence of SEQ ID NO: 100.

29. The antibody or antigen binding fragment thereof of claim 25 or 26, wherein the antibody heavy chain constant region comprises one or more mutations that enhance effector function.

30. The antibody or antigen binding fragment thereof of claim 29, wherein the antibody heavy chain constant region is a human IgG1 heavy chain constant region comprising one of the following mutation(s),
(a) E2331);
(b) G236D;
(c) Gi237D;
(d) P238D;
(e) S239D;
(f) S267E;
(g) H268D;
(h) P271G;
(i) L328Y, L328F, or L328E;
(j) A330R;
(k) I332E;
(l) G236D and H268D;
(m) S239D and H268D;
(n) S239D, H268D, L328Y, and I332E;
(o) P238D and L328E;
(p) G237D, P271G, and A330R;
(q) (1237D, H268D, P271 G, and A330R;
(r) S267E and L328F;
(s) S239D and S267E;
(t) G236D and S276E; or
(u) E233D, G237D, H268D, P271 G, and A330R,
or a combination thereof, wherein the numbering of the constant region is as per the EU index.

31. The antibody or antigen binding fragment thereof of claim 30, wherein the antibody heavy chain constant region is a human IgG1 heavy chain constant region comprising the following mutations: S239D and H268D, wherein the numbering of the constant region is as per the EU index.

32. The antibody or antigen binding fragment thereof of claim 30, wherein the antibody heavy chain constant region is a human IgG1 heavy chain constant region comprising the following mutations: E233D, G237D, H268D, P271G, and A330R, wherein the numbering of the constant region is as per the EU index.

33. The antibody or antigen-binding fragment thereof of any one of claims 1-32, wherein the antibody or antigen-binding fragment thereof is produced in a cell line
(a) having an alpha-1,6-fucosyltransferase (Fut8) knockout; or
(b) line overexpressing β1,4-N-acetylglucosaminyltransferase III (GnT-III) and optionally overexpressing Golgi μ-mannosidase II (ManII).

34. The antibody or antigen-binding fragment thereof of any one of claims 1-33, wherein the antibody or antigen-binding fragment is capable of binding to human PD-1.

35. The antibody or antigen-binding fragment thereof of any one of claims 1-34, wherein the antibody or antigen-binding fragment is capable of binding to cynomolgus PD-1.

36. The antibody or antigen-binding fragment thereof of any one of claims 1-35, wherein the antibody or antigen-binding fragment is capable of blocking PD-1/PD-L1 interaction.

37. The antibody or antigen-binding fragment thereof of any one of claims 1-36, wherein the antibody or antigen-binding fragment is capable of inhibiting T cell activation.

38. The antibody or antigen-binding fragment thereof of any one of claims 1-37, wherein the antibody or antigen-binding fragment is capable of inhibiting T cell proliferation.

38. The antibody or antigen-binding fragment thereof of any one of claims 1-38, wherein the antibody or antigen-binding fragment is capable of inhibiting IFN-γ secretion.

39. The antibody or antigen-binding fragment thereof of any one of claims 1-39, wherein the wherein the antibody or antigen-binding fragment thereof is conjugated to an agent.

41. The antibody or antigen-binding fragment thereof of claim 40, wherein the agent is a cytotoxic agent or label.

42. The antibody or antigen-binding fragment thereof of claim 40, wherein the agent is a therapeutic agent.

43. A composition comprising the antibody or antigen-binding fragment thereof of any one of claims 1-42.

44. The composition of claim 43, wherein the antibody or antigen-binding fragment comprises an Fc region and N-glycoside-linked carbohydrate chains linked to the Fc region, optionally wherein less than 50% of the N-glycoside-linked carbohydrate chains contain a fucose residue.

45. The composition of claim 44, wherein substantially none of the N-glycoside-linked carbohydrate chains contain a fucose residue.

46. A polynucleotide encoding an antibody or antigen binding fragment thereof of any one of claims 1-42.

47. An expression vector comprising the polynucleotide of claim 46.

48. A host cell comprising the polynucleotide of claim 46 or the expression vector of claim 47.

49. The host cell of claim 48, wherein the host cell is a mammalian or insect cell.

50. The host cell of claim 48 or 49, wherein the host cell (a) comprises a Fut8 knockout; and/or (b) overexpresses GnT-III and optionally ManII.

51. A pharmaceutical composition comprising an antibody or antigen binding fragment thereof of any one of claims 1-42, and a pharmaceutically acceptable carrier.

52. A method of treating a disease or disorder in a subject in need thereof, comprising administering to the subject an effective amount of an antibody or antigen binding fragment thereof of any one of claims 1-42, a composition of any one of claims 43-45, or a pharmaceutical composition of claim 51.

53. The method of claim 52, wherein the disease or disorder is an autoimmune disease.

54. The method of claim 52, wherein the disease or disorder is an allergy.

55. The method of claim 52, wherein the disease or disorder is an inflammatory disease.
